(19) United States
(12) Patent Application Publication
(10) Pub. No.: US 2022/0323609 A1
(43) Pub. Date: Oct. 13, 2022

(54) GENE EDITING TO CORRECT ANEUPLOIDIES AND FRAME SHIFT MUTATIONS

(71) Applicant: The Trustees of Columbia University in the City of New York, New York, NY (US)

(72) Inventors: Dietrich EGLI, New York, NY (US); Nathan TREFF, Flanders, NJ (US)

(21) Appl. No.: 17/717,697

(22) Filed: Apr. 11, 2022

Related U.S. Application Data
(63) Continuation-in-part of application No. PCT/US2020/055223, filed on Oct. 12, 2020.
(60) Provisional application No. 63/011,425, filed on Apr. 17, 2020.
(60) Provisional application No. 62/913,647, filed on Oct. 10, 2019.

(51) Int. Cl. A61K 48/00; C12N 15/873; C12N 15/90; C12N 5/073

(52) U.S. Cl. CPC A61K 48/005; C12N 15/873; C12N 15/907; C12N 5/0604; A01K 2227/106; C12N 2310/20; C12N 2800/80

(57) ABSTRACT

The present disclosure relates to using CRISPR-based methods to perform gene editing to correct frame shift mutations in alleles with detectable phenotypes, and to correct aneuploidies.

### CROSS REFERENCE TO RELATED APPLICATION

The present application is a continuation-in-part of PCT Application No. PCT/US2020/055223, which claims priority to U.S. Provisional Applications Nos. 62/913,647 filed on Oct. 10, 2019 and 63/011,425 filed on Apr. 17, 2020, all of which are incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 11, 2022, is named 01001/008388-US1_SL.txt and is 6 kilobytes in size.

### FIELD

The present disclosure relates to using CRISPR-based methods to perform gene editing in to correct aneuploidies embryos and frame shift mutations.

### BACKGROUND

Double-strand breaks (DSBs) stimulate recombination between homologous DNA segments (Jasin and Rothstein, 2013). The targeted introduction of a DSB followed by recombination allows for the precise modification of genomes in model organisms and cell lines, and may also be useful for the correction of disease-causing mutations in the human germ line (Lea and Niakan, 2019). DSBs occur naturally during meiosis, and are repaired through recombination between homologous chromosomes, thereby ensuring genome transmission and genetic diversity in offspring. Recombination between homologs was also recently suggested to occur efficiently in mitotically dividing cells: a DSB at the site of a disease-causing mutation on the paternal chromosome resulted in the loss of the mutation such that approximately half of the resulting embryos carried only the maternal wild-type allele (Ma et al., 2018; Ma et al., 2017). The elimination was presumed to occur through use of the maternal genome as a repair template, resulting in what appeared to be the efficient correction of a pathogenic mutation on the paternal chromosome without mosaicism. This contrasts with frequent mosaicism in previous studies with different cells of the same embryo carrying various edited and non-edited alleles (Liang et al., 2015).

The correction of pathogenic mutations through interhomolog recombination with a lack of mosaicism, if independently confirmed, would have major advantages over other approaches, since such a mechanism of correction does not require the introduction of exogenous nucleic acids and is limited to alleles already present in the human population. However, alternative interpretations of the results have been proposed, including the loss of the paternal allele through large deletions, chromosome loss, or translocations (Adikusuma et al., 2018; Egli et al., 2018).

Furthermore, the physical location of the genomes during the first cell cycle pose a significant limitation to the occurrence of such a recombination event, as maternal and paternal genomes are packaged in separate nuclei and only come together in the same nucleus after the first mitosis at the two-cell stage (Reichmann et al., 2018). Thus, the timing of Cas9-induced breakage and repair is an important determinant of the genetic outcomes with regard to mosaicism and the mechanisms available for repair. Many questions remain regarding DSB repair in human embryos, in particular because of a limited ability to detect complex repair events in a small number of cells.

Aneuploidies due to abnormal chromosome segregation in female meiosis are some of the most frequent problems in human reproduction, resulting in effects such as miscarriage and Down syndrome. Though embryos can be selected and eliminated by aneuploidy testing in IVF clinics prior to implantation, the loss of affected embryos reduces fertility rates. In women of advanced maternal age, the aneuploidy rate increases dramatically, and becomes an almost insurmountable obstacle to reproduction. The development of a method that can correct aneuploidy would therefore be very meaningful.

The correction of disease-causing mutations in human embryos could reduce the burden of inherited genetic disorders in the fetus and newborn and improve the efficiency of fertility treatments for couples with disease-causing mutations in lieu of embryo selection.

Shown herein is the nonmosiac correction of both frame-shift mutations and trisomies using RNA-guided endonucleases in a precise targeted manner.

### SUMMARY

The disclosure herein provides for methods and systems for correcting aneuploidies and frame shift mutations using RNA-guided endonucleases, in particular CRISPR/Cas systems.

In one embodiment, the disclosure provides for methods for correcting an aneuploidy in an embryo comprising introducing into the embryo: (i) at least one guide RNA (gRNA) or DNA encoding at least one guide RNA (gRNA); and (ii) at least one RNA-guided endonuclease or DNA encoding an RNA-guided endonuclease, wherein the endonuclease introduces at least one double-stranded break in a targeted site resulting in the loss or elimination of the extra chromosome.

The method can further comprise culturing the embryo such that each guide RNA directs an RNA-guided endonuclease to a targeted site in the gene where the RNA-guided endonuclease introduces a double-stranded break in the targeted site resulting in a loss of the entire chromosome.

In some embodiments, the RNA-guided endonuclease introduces a single break in the targeted site.

In some embodiments, more than one gRNA is used. In some embodiments, two gRNAs are used. In some embodiments, three gRNAs are used. In some embodiments, four gRNAs are used. In some embodiments, five gRNAs are used. In some embodiments, six gRNAs are used. In some embodiments, seven gRNAs are used. In some embodiments, eight gRNAs are used. In some embodiments, more than eight gRNAs are used.

In some embodiments, one or more gRNAs are designed and used to target opposite sides of the centromere of the chromosome to be eliminated. In some embodiments, two gRNAs are designed and used to target opposite sides of the centromere of the chromosome to be eliminated, e.g., one gRNA targets each opposite side of the centromere. In some embodiments, three gRNAs are designed and used to target opposite sides of the centromere of the chromosome to be eliminated, e.g., one or two gRNAs target each opposite side of the centromere. In some embodiments, four gRNAs are designed and used to target opposite sides of the centromere of the chromosome to be eliminated, e.g., two gRNAs target each opposite side of the centromere. In some embodiments, five gRNAs are designed and used to target opposite sides of the centromere of the chromosome to be eliminated, e.g., two or three gRNAs target each opposite side of the centromere. In some embodiments, six gRNAs are designed and used to target opposite sides of the centromere of the chromosome to be eliminated, e.g., three gRNAs target each opposite side of the centromere. In some embodiments, seven gRNAs are designed and used to target opposite sides of the centromere of the chromosome to be eliminated, e.g., three or four gRNAs target each opposite side of the centromere. In some embodiments, eight gRNAs are designed and used to target opposite sides of the centromere of the chromosome to be eliminated, e.g., four gRNAs target each opposite side of the centromere.

In some embodiments, the gRNA(s) are designed using common single nucleotide polymorphisms (SNPs) flanking the centromere of the chromosome to be targeted. In some embodiments, the SNP is within about 1 to about 5 Mb from the centromere. In some embodiments, the SNP is within about 3 Mb of the centromere. In some embodiments, the more than one gRNA being used is designed using SNPs from opposite sides of the centromere such that at least one gRNA targets opposite sides of the centromere.

In some embodiment, the RNA-guided endonuclease is a Cas nuclease. In some embodiments, the Cas nuclease is Cas9.

In some embodiments, to determine the chromosome which is causing the aneuploidy, preimplantation genetic screening of the embryo is performed. In some embodiments, there is a history of aneuploidy either in the parents or a sibling of the embryo. In some embodiments, there is a history of miscarriage by the mother of the embryo. In some embodiments, the mother of the embryo is older than 35 years old. In some embodiments, the mother of the embryo is older than 40 years old. In some embodiments, the mother of the embryo is older than 45 years old.

In some embodiments, the parent carries a Robertsonian translocation, such as an isodisomy 21, or another chromosomal variant. These variants can lead to frequent and predictable missegregation in meiosis and trisomies.

In some embodiments, the aneuploidy is a trisomy. In some embodiments, the aneuploidy is trisomy 8, trisomy 9, trisomy 13, trisomy 16, trisomy 18, trisomy 21, trisomy 22, trisomy X or trisomy Y.

In some embodiments, the molecules are introduced into the embryo by microinjection. Typically, the embryo is a fertilized one-cell or two-cell stage embryo.

A further embodiment of the present disclosure is a method for correcting or modifying a frame shift mutation in an allele in an embryo comprising introducing into the embryo: (i) at least one guide RNA or DNA encoding at least one guide RNA that hybridizes to the mutated allele; and (ii) at least one RNA-guided endonuclease or DNA encoding an RNA-guided endonuclease, wherein the endonuclease introduces a double-stranded break in a targeted site on the mutated allele resulting in the correction or modification of the frame shift mutation.

A further embodiment of the present disclosure is a method for correcting or modifying a frame shift mutation in an allele in a cell of a subject comprising contacting the cell with at least one type of vector comprising: (i) a first sequence encoding a guide RNA that hybridizes to the mutated allele; and (ii) a second sequence encoding at least one RNA-guided endonuclease, wherein the endonuclease introduces a double-stranded break in a targeted site on the mutated allele resulting in the correction or modification of the frame shift mutation.

Non-limiting examples of cells include animal cell, mammalian cells, canine cells, feline cells, equine cells and human cells.

A further embodiment of the present disclosure is a method for correcting or modifying a frame shift mutation in an allele in a subject, comprising administering to the subject a therapeutically effective amount at least one type of vector comprising: (i) a first sequence encoding a guide RNA that hybridizes to the mutated allele; and (ii) a second sequence encoding at least one RNA-guided endonuclease, wherein the endonuclease introduces a double-stranded break in a targeted site on the mutated allele resulting in the correction or modification of the frame shift mutation.

In some embodiments, the subject is a fetus. In some embodiments, the subject is a newborn. In some embodiments, the subject is a child. In some embodiments, the subject is an adult.

The current methods and systems to correct frame shift mutations can be used for any mutant homozygous alleles which have a known phenotype. Since the phenotype is known, a father or mother or the subject who has the mutated allele would have the corresponding phenotype. Thus, the identification of the allele is within the skill of the art.

Phenotypes, i.e., disorders or diseases, that can be corrected using the methods and systems of the current disclosure include but are not limited to mutations in the EYS locus, retinitis pigmentosa and Tay Sachs disease.

In some embodiments, the correction is made in a nonmosaic manner. In some embodiments, the correct is made in a mosaic manner.

In some embodiments, the double-stranded break in the allele is repaired by a MMEJ repair process. In some embodiments, the double stranded break in the allele is repaired by a NHEJ repair process.

In some embodiments, the gRNA is designed to target the mutated but not wild-type allele. In some embodiments, the mutated allele is the paternal allele. In some embodiments, the mutated allele is the maternal allele. In some embodiments, the mutated and wild-type allele differ at the PAM sequence motif. In some embodiments, the gRNA is designed such that placement results in cleavage between two identical regions of nucleotides in the mutated allele, which defines the sites of micro-homology. In some embodiments, these regions are about 2 bps to about 3 bps. In some embodiments, these regions are about 3 bps to about 5 bps.

In some embodiments, these regions are about 5 bps to about 8 bps.

In some embodiment, the RNA-guided endonuclease is a Cas nuclease. In some embodiments, the Cas nuclease is Cas9.

In some embodiments, the RNA-guided endonuclease and gRNA are introduced into the cell or embryo in the form of a ribonucleoprotein complex comprising the endonuclease complexed to least one gRNA. Preparation of such RNP complexes are known in the art or can be obtained commercially.

In some embodiments, the RNP is introduced into an oocyte at the same time as a sperm cell. This can be accomplished using intracytoplasmic sperm injection (ICSI).

In some embodiments, genotyping an oocyte and sperm donor is performed to determine the location of the mutated allele and the specific frame shift mutation on the mutated allele, prior to the introduction of the at least one guide RNA or DNA encoding at least one guide RNA, and the RNA-guided endonuclease, or DNA encoding an RNA-guided endonuclease the embryo.

In some embodiments, the Cas9/gRNA is introduced with a vector used for gene therapy in adult somatic cells.

The present disclosure also provides for systems for carrying out any of the disclosed methods.

### BRIEF DESCRIPTION OF THE DRAWINGS

For the purpose of illustrating the invention, certain embodiments of the invention are depicted in drawings. However, the invention is not limited to the precise arrangements and instrumentalities of the embodiments depicted in the drawings.

FIG. 1: Efficient end joining within the first cell cycle after Cas9 RNP injection at fertilization. FIG. 1A is a schematic of the human EYS locus with the paternal homozygous EYS^{2265fs} mutation. Alignment of paternal and maternal alleles, gRNA target, primers indicated by arrowheads, and flanking SNPs are indicated. Flanking primers (top arrowheads) were used for amplification and sequencing of the mutation site and linked SNPs within a single PCR product. The internal primers (bottom arrowheads) were on target NGS, as well as for Sanger sequencing. CEN=centromere, TEL=telomere. Direction of EYS transcription is towards the centromere, indicated by arrow. FIG. 1A also shows the results of Sanger sequencing of oocyte and sperm donor with homozygous different flanking SNPs. FIG. 1B is a schematic of gRNA specificity testing in embryonic stem cells (ESC) with the same chromosomal constitution as the fertilized zygote. 48 h after Cas9-GFP nucleofection, cells are harvested and used for on-target NGS of the mutation site and rs66502009. FIG. 1C is a graph of the results of the on-target NGS of the mutation site and rs66502009 read quantification of edited and original alleles 48 h after Cas9-GFP nucleofection in seven independent experiments of two cell lines. Pat=paternal allele, mat=maternal allele. FIG. 1D is a graph of the type and frequency of indels in human pluripotent stem cells evaluated using on-target NGS. FIG. 1E is a graph of the type and frequency of indels in human pluripotent stem cells evaluated by colony picking and Sanger sequencing. FIG. 1F is a schematic of DSB repair events after Cas9 cleavage. Cas9 cleaves between two regions of microhomology. Alternate products of microhomology-mediated end joining (MMEJ) obtained in human embryos is also shown. Nonhomologous end joining can result in reading frame restoration due to insertion of an A due to the Cas9 overhang. FIG. 1G is a schematic of editing outcomes when mutant sperm is injected into the cytoplasm together with a gRNA and RNP complex of Cas9 at the MII stage. FIG. 1H is a schematic of the injection performed after fertilization at the 2-cell stage. FIG. 1I is a graph of the quantification of type and frequency of indels of combined data from MII and 2-cell stage injections. FIG. 1J is a graph of the frequency of ESC clones or embryos with heterozygous indels versus clones or embryos with loss of paternal alleles and an EYS^{wt} genotype. Statistical analysis was performed using Fisher's exact test. RNP=ribonucleoprotein.

FIG. 2: DSB repair occurs during the first cell cycle and independent of the maternal genome. FIG. 2A is a schematic of androgenesis where a single sperm is injected into an enucleated oocyte, resulting in a 1PN zygote, followed by collection at 20 h in the first cell cycle for genotyping and the results of genotyping by Sanger sequencing at 20 h in the first cell cycle. FIG. 2B is a schematic of fertilization resulting in a zygote with both maternal and paternal genomes, with nuclei isolated and separated to two different tubes for Sanger analysis also shown. FIG. 2C shows the results of ‘standard’ genotyping analysis where both nuclei of the zygote 20 hrs post Cas9 RNP were collected in the same tube with genotyping. FIG. 2D is a graph of the results of on target paternal genotyping. FIG. 2E shows a parent of origin analysis of a whole 2PN zygote (without polar body) with an EYS^{wt} genotype through SNP array. Note paternal, maternal and heterozygous SNPs (blue) throughout chromosome 6. On top, shown is the chromosomal location of the Cas9 target site at the EYS locus. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots. up=chr6:1-64.7 Mb, and down=chr6:64.7 Mb-telomere. The increased signal around chr6:30 Mb is in the HLA region, which shows increased background signal. FIG. 2F shows a parent of origin analysis of separated nuclei isolated from 2PN zygotes through SNP array. Note that the paternal nucleus (pMII-5) and the maternal nucleus (mMII-5) contain alleles of the expected origin throughout chromosome 6. On top, shown is the chromosomal location of the Cas9 target site at the EYS locus. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots. up=chr6:1-64.7 Mb, and down=chr6:64.7 Mb-telomere. The increased signal around chr6:30 Mb is in the HLA region, which shows increased background signal. FIG. 2G is a graph of a summary of all Sanger genotyping results of the paternal EYS locus at the 1-cell stage at 20 h. The number of maternal and paternal nuclei is unequal because androgenesis excludes the maternal genome. FIG. 2H is a schematic of a model for failure to detect a paternal allele with parental alleles indicated for zygotes 5 and 7. PCR requires an intact DNA strand; a DSB interferes with the amplification of primers flanking the Cas9 cut site. In the presence of a maternal allele, the zygote appears as EYS^{wt}. Arrows and arrowheads indicates primer pairs. CEN=centromere. TEL=telomere. pat=paternal, mat=maternal.

FIG. 3: Chromosome loss in embryos with a ‘wild type’ genotype. FIG. 3A is a schematic of ICSI at the MII stage with Cas9 RNP followed by development to the cleavage and blastocyst stages with analysis at each stage. Analysis at the cleavage stage involves harvesting of all cells and is incompatible with further development; these are different embryos. FIG. 3B shows the result of the on-target analysis by Sanger sequencing for embryos and embryonic stem cells. Shown is the percentage of embryos with the indicated genotypes. FIG. 3C is the heterozygosity analysis on chromosome 6 by SNP array for TE biopsy embryo D (blastocyst with a heterozygous indel which also gave rise to an ESC line) and Sanger sequencing profiles for the mutation site and a SNPs informative of parental origin centromeric to the cut site in the same embryo. On top, shown is the chromosomal location of the Cas9 target site at the EYS locus. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots. Plot 2 (grey) indicates copy number through signal intensity quantification, with flanking sides of rs758109813 shaded in lighter or darker grey. up=chr6:1-64.7 Mb, and down=chr6:64.7 Mb-telomere. FIG. 3D is the heterozygosity analysis on chromosome 6 by SNP array for TE biopsy embryo E (blastocyst shown with its EYS^{wt} Sanger genotype). On top, shown is the chromosomal location of the Cas9 target site at the EYS locus. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots. Plot 2 (grey) indicates copy number through signal intensity quantification, with flanking sides of rs758109813 shaded in lighter or darker grey. up=chr6:1-64.7 Mb, and down=chr6:64.7 Mb-telomere. FIG. 3E is the heterozygosity analysis on chromosome 6 by SNP array for TE biopsy of a trophectoderm of another blastocyst embryo F. On top, shown is the chromosomal location of the Cas9 target site at the EYS locus. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots. Plot 2 (grey) indicates copy number through signal intensity quantification, with flanking sides of rs758109813 shaded in lighter or darker grey. up=chr6:1-64.7 Mb, and down=chr6:64.7 Mb-telomere. No ESC lines were obtained from the ICM. FIG. 3F is a SNP array analysis of embryo 1, an embryo without the paternal EYS^{rs2265fs} allele. For each panel, the biopsied embryo is indicated by a schematic at the top, and the number of cells successfully isolated and analyzed by SNP array is indicated with the number of tubes. The plots show paternal allele frequency (top plot) and copy number (grey plot). Only SNPs in which the maternal genotype was homozygous for one allele (red) and the paternal genotype was homozygous for the other allele (green) were included. Blue indicates a heterozygous (normal) embryo genotype. Quantification of allelic frequencies is shown above the plots. up=chr6:1-64.7 Mb, and down=chr6:64.7 Mb-telomere. FIG. 3G is a SNP array of embryo C, an embryo without the paternal EYS^{rs2265fs} allele. For each panel, the biopsied embryo is indicated by a schematic at the top, and the number of cells successfully isolated and analyzed by SNP array is indicated with the number of tubes. The plots show paternal allele frequency (top plot) and copy number (grey plot). Only SNPs in which the maternal genotype was homozygous for one allele (red) and the paternal genotype was homozygous for the other allele (green) were included. Blue indicates a heterozygous (normal) embryo genotype. Quantification of allelic frequencies is shown above the plots. up=chr6:1-64.7 Mb, and down=chr6:64.7 Mb-telomere. Sanger sequencing calls of rs1631333 were validated with selected on-target deep sequencing.

FIG. 4: Mosaicism, interhomolog recombination and chromosome loss after injection into a two-cell stage embryo. FIG. 4A is a schematic of the experiment. A human oocyte is injected with EYS^{2265fs} mutant sperm and Cas9 RNP is injected at the 2-cell stage, at 30-35 h post ICSI, followed by analysis of individual cells at the cleavage stage. FIG. 4B is the results of Sanger sequencing profiles of different blastomeres of the same embryo. FIG. 4C shows the results of on-target NGS of embryo samples at the mutation site (rs758109813) and the linked SNP rs66502009, na=not applicable for samples without identifying parental SNP rs6652009. Em=embryo FIG. 4D is a graph of the quantification of the percentage of cells with indicated genotypes determined by Sanger sequencing. FIG. 4E is a schematic of the cell division products observed after a single cell cycle post Cas9 RNP injection. Two cells were successfully analyzed by SNP array, the cleavage products of the second injected cell that did not divide (dotted line), was not. FIG. 4F shows SNP arrays and Sanger sequencing of SNP rs1631333 for sister blastomeres, both EYS^{wt} in on-targeting sequencing but with different chromosome 6 content. Shown is the chromosomal location of the Cas9 target site at the EYS locus and the SNP rs1631333 informative of parental origin. The SNP array plots show paternal allele frequency. Only SNPs in which the maternal genotype was homozygous for one allele (red) and the paternal genotype was homozygous for the other allele (green) were used for analysis. Quantification of allelic frequencies is shown above the plots and in Table S4. Plot 2 (grey) indicates copy number through signal intensity quantification, with flanking sides of rs758109813 shaded in lighter or darker grey. up=chr6:1-64.7 Mb, and down=chr6:64.7 Mb-telomere. CEN=centromere. Pat=paternal. Mat=maternal. FIG. 4G shows the quantification of aneuploidies according to parental origin. Statistical analysis was performed using Fisher's exact test. FIG. 4H shows a schematic of the cell division products observed after a single cell cycle post Cas9 RNP injection. Three cells/fragments were successfully analyzed. Dotted circle indicates another cell of the same 2-cell embryo without a result. FIG. 4I-4K shows Sanger sequencing of rs1631333 and corresponding SNP arrays for two different cells and one cytoplasmic. Shown is the chromosomal location of the Cas9 target site at the EYS locus and the SNP rs1631333 informative of parental origin. The plots show paternal allele frequency and copy number analysis (grey). Only SNPs in which the maternal genotype was homozygous for one allele (red) and the paternal genotype was homozygous for the other allele (green) were used for analysis. Blue indicates a heterozygous (normal) embryo genotype. Quantification of allelic frequencies is shown above the plots. up=chr6:1-64.7 Mb, and down=chr6:64.7 Mb-telomere. The area centromeric of the EYS gene and 6p are shaded in a dark grey, 6q telomeric of EYS in a lighter grey. FIG. 4I shows a cell with a loss of chromosome 6q. FIG. 4J shows a cell with a loss of chromosome 6p and a gain of chromosome 6q. FIG. 4K shows a ‘cell’ with only chromosome 6p without any other genomic DNA. The signal on the q arm is background/noise. Note that the cleavage products add up to 2 copies for each 6p and 6q arm. CEN=centromere.

FIG. 5: Aneuploidy and indels due to Cas9 off-target activity on chromosome 16. FIG. 5A are graphs of the number of cells with segmental aneuploidies of maternal or paternal origin for each chromosome 1-22. Analysis includes all 38 blastomeres obtained after MII or 2-cell Cas9 RNP injections analyzed through SNP karyotyping. FIG. 5B shows off-target site with 2 mismatches on chromosome 16923.1, concordant with the cytological location of segmental aneuploidies. Underlined are regions of microhomology, which lead to recurrent 5 bp and 8 bp deletions. Figs. C-F show the results of an analysis of off-target activity on chromosome 16923.1. FIG. 5C is a graph of the frequency of off-target indels in 27 blastomeres, and frequency of off-target segmental aneuploidies in 37 blastomeres obtained after MII or 2-cell Cas9 RNP injections. FIG. 5D is a graph of the incidence of mosaicism at the off-target site in blastomeres after Cas9 injection at fertilization. FIG. 5E is a graph of the indels in single haploid isolated nuclei at the 1-cell stage at 20 h post fertilization and Cas9 RNP injection. FIG. 5F shows SNP array analysis of sister blastomeres a single cell cycle after Cas9 RNP injection at the 2-cell stage. Dotted circle indicates another cell of the same 2-cell embryo without a result. SNPs in which the maternal genotype was homozygous for one allele (red) and the paternal genotype was homozygous for the other allele (green) were used. Blue indicates a heterozygous (normal) embryo genotype. Plot 2 (blue) indicates copy number. Corresponding Sanger profiles at the off-target location are provided on the right. Note the apparent ‘homozygosity’ of the indel.

FIG. 6: Mosaic correction of a trisomy 16 embryo. FIG. 6A is a schematic of the experiment. Fertilized zygotes have polar body 1 and polar body 2 removed for genotyping. FIG. 6B shows a bar diagram of the copy number analysis across the genome of one embryo designated B2. Note the loss of one chromosome 13 in polar body 1, and the gain in polar body2, resulting in a normal chromosome 13 complement in the embryo. In contrast, chromosome 16 is lost in polar body 2, resulting in a gain in the embryo. FIG. 6C shows a bar diagram of the copy number analysis across the genome of four embryo cells from embryo B2 on day2 of development. FIG. 2D shows the copy number analysis across chromosomal arms in blastomere 3 with imbalanced copies of 16q and 16p arm (top) and in blastomere 4 which is normal (bottom), after injection with CRISPR/Cas9 targeting the pericentromeric p arm of chromosome 16 at the pronuclear stage.

### DETAILED DESCRIPTION

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

The term “cell” as used herein may refer to either a prokaryotic or eukaryotic cell, optionally obtained from a subject or a commercially available source.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” or homology (equivalence or equivalents) to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “target”, “targets” or “targeting” refers to partial or no breakage of the covalent backbone of polynucleotide. In one embodiment, a deactivated Cas protein (or dCas) targets a nucleotide sequence after forming a DNA-bound complex with a guide RNA. Because the nuclease activity of the dCas is entirely or partially deactivated, the dCas binds to the sequence without cleaving or fully cleaving the sequence. In some embodiment, targeting a gene sequence or its promoter with a dCas can inhibit or prevent transcription and/or expression of a polynucleotide or gene.

The term “Cas9” refers to a CRISPR associated endonuclease referred to by this name. Non-limiting exemplary Cas9s are provided herein, e.g., the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”); Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, et al. 2014. Nature biotechnology 32(12):1262-7, Mohr, et al. 2016. FEBS Journal 3232-38, and Graham, et al. 2015. Genome Biol. 16:260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, et al. 2016. J of Biotechnology 233:74-83). As used herein, a biological equivalent of a gRNA includes but is not limited to polynucleotides or targeting molecules that can guide a Cas9 or equivalent thereof to a specific nucleotide sequence such as a specific region of a cell's genome.

The term “embryo” refers to the early stage of development of a multicellular organism. In general, in organisms that reproduce sexually, embryonic development refers to the portion of the life cycle that begins just after fertilization and continues through the formation of body structures, such as tissues and organs. Each embryo starts development as a zygote, a single cell resulting from the fusion of gametes (i.e., fertilization of a female egg cell by a male sperm cell). In the first stages of embryonic development, a single-celled zygote undergoes many rapid cell divisions, called cleavage, to form a blastula.

The term “mosaicism” is defined as the presence of two or more populations of cells with different genotypes in one individual who has developed from a fertilized egg.

### Abbreviations

CEN—centromere
TEL—telomere
RNP—ribonucleoprotein
MMEJ—microhomology-mediated end joining
NHEJ—nonhomologous end joining
INDELs—insertions and/or deletions

The genome bestowed at fertilization determines much of our health as adults. While genetic mutations may be corrected after birth using somatic gene therapy, the efficacy of this approach is dependent on the number of cells that can be edited. In addition, the mutation will still be passed on to the next generation. In contrast, gene editing in the embryo will alter the genome of all cells and can result in a durable therapeutic effect.

An important requirement for clinical application is the ability to predict outcomes. Mosaicism prevents inferring the genotype of the fetus from trophectoderm and is thus incompatible with clinical use. Therefore, the application of CRISPR/Cas9 prior to the first round of DNA replication in the embryo is meaningful. Shown herein is the injection of CRISPR/Cas9 at fertilization with the sperm. It was found that in most, but not all instances, the outcome is non-mosaic editing and a single modified paternal allele was present, and hence occurred before replication of gene being repaired, EYS.

Double-strand break repair occurred predominantly through nonhomologous end joining (NHEJ) or microhomology-mediated end joining (MMEJ). The placement of the guide RNA at EYS^{2265fs} resulted in a cut between 3 nucleotides of microhomology, leading to the elimination of 5 nucleotides through MMEJ as the most common repair event. The deletion of 5 nucleotides restored the reading frame of the EYS^{2265fs} allele through the loss of 2 amino acids relative to the wild type allele in the first of five lamin AG domains (Abd El-Aziz et al., 2008). The reading frame was also restored through the insertion of a single A nucleotide by nonhomologous end joining, resulting in two amino acid transitions relative to the wild type allele. These recurrent 1 bp insertions are the consequence of filling in 1 bp overhangs created by Cas9 cutting (Jasin, 2018; Lemos et al., 2018). As disease causing missense mutations in EYS map to the 4^{th} and 5^{th} lamin AG domains (Khan et al., 2010), restoring the frame restores function.

Surprisingly, a frequent outcome of a single Cas9-induced double-strand break in human embryos at the EYS locus is the loss of the long arm of chromosome 6. The loss of both the long arm 6q, the short arm 6p, as well as of the entire chromosome in blastomeres and trophectoderm biopsies were found. Embryos with an EYS^{wt} genotype that were tested for aneuploidies using SNP arrays showed aneuploidies of chromosome 6; by contrast, embryos with heterozygous edits EYS^{wt/indel} did not. While segmental aneuploidies with a breakpoint at the EYS locus are readily attributed to Cas9 cleavage, whole chromosome errors can occur by either mitotic errors or when uniform in all cells of the embryo, through meiotic segregation errors. However, meiotic chromosome losses are predominantly of maternal origin, and spontaneous losses of chromosome 6 are relatively infrequent (Franasiak et al., 2014). In contrast, in the samples of this study, whole chromosome losses were of paternal origin, and occurred after fertilization. Different cells of the same embryo showed both whole chromosome loss, as well as mirroring losses of the long or the short arms of chromosome 6. Therefore, pericentromeric cleavage at the EYS locus destabilizes the entire chromosome and results in both segmental as well as whole chromosome loss.

This study shows that a DSB induced by even a single gRNA can result in the allele-specific removal of a chromosome in human embryos. Previous studies in mouse embryos demonstrated elimination of sex chromosomes by targeting Cas9 to centromeric repeats of the Y chromosome, or to multiple locations on the same chromosome (Adikusuma et al., 2017; Zuo et al., 2017).

Such induction of allele-specific chromosome loss has clinical applications. Aneuploidies caused by abnormal meiosis are common in human oocytes, which is a major obstacle to fertility treatments and a cause for congenital abnormalities (Hassold and Hunt, 2001). Monosomic chromosomal gains are estimated to occur in about 5% of human oocytes (McCoy et al., 2015), which shown herein are amenable to correction by Cas9. The placement of one or more gRNAs on both sides of the centromere may further increase the frequency of whole chromosome loss and thereby allow efficient allele specific correction of trisomies in human embryos.

### Methods and Systems of Correcting Aneuploidies in Embryos

One aspect of the present disclosure encompasses a method for correcting an aneuploidy in an embryo comprising introducing into the embryo: (i) at least one guide RNA (gRNA) or DNA encoding at least one guide RNA (gRNA); and (ii) at least one RNA-guided endonuclease or DNA encoding an RNA-guided endonuclease. In some embodiments, the aneuploidy is an extra chromosomal aneuploidy, i.e., embryo has 47 rather than 46 chromosomes. The method further comprises culturing the embryo such that each guide RNA directs an RNA-guided endonuclease to a targeted site in the gene where the RNA-guided endonuclease introduces a double-stranded break in the targeted site resulting in a loss of the entire chromosome. In some embodiments, the RNA-guided endonuclease introduces a single break in the targeted site.

In some embodiments, one or more gRNAs are designed and used to target opposite sides of the centromere of the chromosome to be eliminated. In some embodiments, two gRNAs are designed and used to target opposite sides of the centromere of the chromosome to be eliminated, e.g., one gRNA targets each opposite side of the centromere. In some embodiments, three gRNAs are designed and used to target opposite sides of the centromere of the chromosome to be eliminated, e.g., one or two gRNAs target each opposite side of the centromere. In some embodiments, four gRNAs are designed and used to target opposite sides of the centromere of the chromosome to be eliminated, e.g., two gRNAs target each opposite side of the centromere. In some embodiments, five gRNAs are designed and used to target opposite sides of the centromere of the chromosome to be eliminated, e.g., two or three gRNAs target each opposite side of the centromere. In some embodiments, six gRNAs are designed and used to target opposite sides of the centromere of the chromosome to be eliminated, e.g., three gRNAs target each opposite side of the centromere. In some embodiments, seven gRNAs are designed and used to target opposite sides of the centromere of the chromosome to be eliminated, e.g., three or four gRNAs target each opposite side of the centromere. In some embodiments, eight gRNAs are designed and used to target opposite sides of the centromere of the chromosome to be eliminated, e.g., four gRNAs target each opposite side of the centromere.

In some embodiments, the gRNA is designed using common single nucleotide polymorphisms (SNPs) flanking the centromere of the chromosome to be targeted. In some embodiments, the SNP is within about 1 to about 5 Mb from the centromere. In some embodiments, the SNP is within about 3 Mb of the centromere. It is within the skill of the art to design one or more gRNAs which target these known SNPs flanking the centromeres of particular chromosome. Alternatively, gRNAs can be obtained commercially.

The guide RNA is designed to overlap an allele that is specific for the gained chromosome. The location of the difference should be as close to the PAM site as possible, ideally within 5, or also within 10 nucleotides from the PAM site. A guide RNA can be tested for its specificity to the SNP by in vitro digestion of PCR products containing the different alleles. The specificity test can also be done in cell lines containing heterozygous for the different SNPs and analysis of indel frequency.

To determine the chromosome which is causing the aneuploidy preimplantation genetic screening (PGS) of embryos can be performed in a clinical setting. Such PGS is done routinely. In some embodiments, there is a history of aneuploidy either in the parents or a sibling. In some embodiments, there is a history of miscarriage by the mother. In some embodiments, the mother is older than 35 years old. In some embodiments, the mother is older than 40 years old. In some embodiments, the mother is older than 45 years old.

In some embodiments, the guide RNAs can be introduced into the embryo as a RNA molecule in a complex with Cas9 protein. The RNA molecule can be transcribed in vitro. Alternatively, the RNA molecule can be chemically synthesized.

In other embodiments, the guide RNAs can be introduced into the embryo as a DNA molecule. In such cases, the DNA encoding the guide RNA can be operably linked to promoter control sequence for expression of the guide RNA in the cell or embryo of interest. For example, the RNA coding sequence can be operably linked to a promoter sequence that is recognized by RNA polymerase III (Pol III). Examples of suitable Pol III promoters include, but are not limited to, mammalian U6 or H1 promoters. In some embodiments, the RNA coding sequence is linked to a human U6 promoter. In other exemplary embodiments, the RNA coding sequence is linked to a human H1 promoter.

The DNA molecule encoding the guide RNA can be linear or circular. In some embodiments, the DNA sequence encoding the guide RNA can be part of a vector. Suitable vectors include plasmid vectors, phagemids, cosmids, artificial/mini-chromosomes, transposons, and viral vectors. In some embodiments, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some embodiments, the DNA molecules encoding different guide RNAs are part of separate molecules (e.g., different vectors). In some embodiments, the DNA molecules encoding the different guide RNAs are part of the same molecule (e.g., same vector).

In embodiments in which both the RNA-guided endonuclease and the guide RNAs are introduced into the cell as DNA molecules, each can be part of a separate molecule (e.g., one vector containing endonuclease coding sequence and a second vector containing guide RNA(s) coding sequence) or both can be part of the same molecule (e.g., one vector containing coding (and regulatory) sequence for both the endonuclease and the guide RNA).

In some embodiments, the RNA-guided endonuclease and gRNAs are introduced into the cell or embryo in the form of a complex comprising the endonuclease complexed to least one gRNA. Preparation of such ribonucleotide protein (RNP) complexes are known in the art or can be obtained commercially.

The RNA-targeted endonuclease(s) (or encoding nucleic acid), and the guide RNA(s) (or encoding DNA), can be introduced into an embryo by a variety of means. In some embodiments, the embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001).

In other embodiments, the molecules are introduced into the embryo by microinjection. Typically, the embryo is a fertilized one-cell or two-cell stage embryo.

The method further comprises maintaining the embryo under appropriate conditions such that the guide RNA(s) directs the RNA-guided endonuclease(s) to the targeted site(s) in the allele, and the RNA-guided endonuclease(s) introduce at least one double-stranded break in the allele.

An embryo can be cultured in vitro (e.g., in cell culture). Typically, the embryo is cultured at an appropriate temperature and in appropriate media with the necessary O₂/CO₂ ratio to allow the expression of the RNA endonuclease and guide RNA, if necessary. Suitable non-limiting examples of media include M2, M16, KSOM, BMOC, and HTF media. A skilled artisan will appreciate that culture conditions can and will vary.

The current methods and systems to correct aneuploidies can be used for any aneuploidy which is caused by an extra chromosome, including any of the 46 chromosomes.

Such known aneuploidies, otherwise known as trisomies, include but are not limited to those listed in Table 1.

TABLE 1
Exemplary Trisomies

| Chromosome | Phenotype |
|---|---|
| 8 | Warnany Syndrome |
| 9 | Miscarriage |
| 13 | Patau Syndrome |
| 16 | Miscarriage |
| 18 | Edward’s Syndrome |
| 21 | Down’s Syndrome |
| 22 | Miscarriage |
| X | Klinefelter Syndrome |
| Y | Jacob Syndrome |
| All other chromosome | Miscarriage, pregnancy failure |

In some embodiments, the clinical work flow of using the method herein to correct aneuploidy in an embryo would be as follows.
1. The first step is designing guide RNAs specific for common SNPS flanking the centromere (e.g., within 3 Mb from the centromere) of each chromosome, could be designed using available data on SNPs. Available SNPs can be obtained from dbSNP (www.ncbi.nlm.nih.gov/projects/SNP), SNPs which are particularly useful are those near the centromeres of the chromosomes. In some embodiments, gRNA would be designed in a company or commercial setting. In some embodiments, gRNA would be designed in a clinic, such as a fertility clinic.
   Alleles with the greatest variation are prioritized, as it increases the chance that one guide RNA will required frequently. Allele-specific gRNAs can be vetted for specificity using an in vitro digestion assay using Cas9-gRNA complex designed for either allele. Specificity of cleavage can also be evaluated in cultured cells, such as a collection of pluripotent stem cells.
2. The next step would be the collection of somatic cells from both an egg and sperm donor. This would typically be performed in a fertility clinic or other clinical setting. The cells are sent to a genotyping facility, which may be in a commercial setting and may be a clinical setting.
   Fertilization of the eggs is performed and both the both the first and the second polar body are isolated for genotyping. The first polar body is obtained at fertilization, and the second polar body between 4-20 h post fertilization (e.g., at fertilization check on day 1). The fertilized egg can be frozen until genotyping is complete.
3. Next, genotyping of both sperm donor and oocyte donor for single nucleotide polymorphisms (SNPs) is performed using either whole genome sequencing or SNP arrays, such as Affymetrix or Illumina SNP array, or another commercially available SNP platform. The goal of this genotyping is to identify SNPs that are heterozygous in the oocyte donor, and not present in the sperm donor. The number of SNPs that meet these criteria are expected to be several thousand. A typical SNP array evaluates about 1 million SNPs, which are highly polymorphic between individuals.
   Genotyping of the two polar bodies involves whole genome amplification, e.g., using the REPLI-g kit, or another commercially available whole genome amplification kit. Genotyping will tell whether the embryo is aneuploid, and what aneuploidy it carries, on which chromosome, whether it is a gain or a loss, and which parental chromosome(s) are causing the aneuploidy.
4. The next step is to select SNPs within 1-5 Mb of the centromere that are specific to the chromosome gained. The identity of the gain is learned through its absence in the polar body one and two. The gRNAs designed in the first step can be used or gRNA designed specifically for the SNPs selected in this step. The validated gRNAs are complexed to Cas9 (or another endonuclease) and introduced into the embryo by any method described herein. In some embodiments, a commercial entity which has designed the gRNA based upon the SNPs prepares the gRNA(s) and endonucleases for introduction into the embryos and delivers the gRNA/endonucleases to a fertility clinic or other clinical setting. In some embodiments, the fertility clinic prepares the gRNA and endonucleases.
5. Last, the embryos are thawed and those with chromosomal gain(s) are injected with the specific guide RNA after thawing. The following steps are routine clinical practice. The egg is cultured to the blastocyst stage and a tropectoderm biopsy is performed to determine the karyotype. Embryos with a normal karyotype are implanted. The blastocyst may be frozen prior to implantation.

The current disclosure also includes systems comprising RNA-guided endonucleases (e.g., Cas9) or DNA encoding the endonuclease, gRNA(s) (or DNA encoding the gRNAs) designed to target the extra chromosomes including at least one gRNA targeting a SNP flanking each opposite side of the centromere of the extra chromosome, and/or delivery systems of such components such as vectors and RNPs comprising these components.

### Methods and Systems of Modifying or Correcting Frame Shift Mutations

A further embodiment of the present disclosure is a method for correcting or modifying a frame shift mutation in an allele in an embryo comprising introducing into the embryo: (i) at least one guide RNA or DNA encoding at least one guide RNA that hybridizes to the mutated allele; and (ii) at least one RNA-guided endonuclease or DNA encoding an RNA-guided endonuclease, wherein the endonuclease introduces a double-stranded break in a targeted site on the mutated allele resulting in the correction or modification of the frame shift mutation.

A further embodiment of the present disclosure is a method for correcting or modifying a frame shift mutation in an allele in a cell of a subject comprising contacting the cell with at least one type of vector comprising: (i) a first sequence encoding a guide RNA that hybridizes to the mutated allele; and (ii) a second sequence encoding at least one RNA-guided endonuclease, wherein the endonuclease introduces a double-stranded break in a targeted site on the mutated allele resulting in the correction or modification of the frame shift mutation.

Non-limiting examples of cells include animal cell, mammalian cells, canine cells, feline cells, equine cells and human cells.

A further embodiment of the present disclosure is a method for correcting or modifying a frame shift mutation in an allele in a subject, comprising administering to the subject a therapeutically effective amount at least one type of vector comprising: (i) a first sequence encoding a guide RNA that hybridizes to the mutated allele; and (ii) a second sequence encoding at least one RNA-guided endonuclease, wherein the endonuclease introduces a double-stranded break in a targeted site on the mutated allele resulting in the correction or modification of the frame shift mutation.

In some embodiments, the subject is an embryo. In some embodiments, the subject is a fetus. In some embodiments, the subject is a newborn. In some embodiments, the subject is a child. In some embodiments, the subject is an adult.

In some embodiments, the correction is made in a nonmosaic manner. In some embodiments, the correct is made in a mosaic manner.

In some embodiments, the double-stranded break in the allele is repaired by a MMEJ repair process. In some embodiments, the double stranded break in the allele is repaired by a NHEJ repair process.

In some embodiments, the frame shift mutation is corrected by deleting nucleotides from the mutated allele. In some embodiments, the frame shift mutation is corrected by adding nucleotides to the mutated allele. The current methods and systems to correct frame shift mutations can be used for any mutant homozygous alleles which have a known phenotype. Since the phenotype is known, a father or mother or subject who has the mutated allele would have the corresponding detectable phenotype. Thus, the identification of the allele corresponding to the phenotype is within the skill of the art.

Phenotypes, i.e., disorders or diseases, that can be corrected using the methods and systems of the current disclosure include but are not limited to mutations in the EYS locus, retinitis pigmentosa and Tay Sachs disease.

In some embodiments, the gRNA is designed to target the mutated but not the wild-type allele. In some embodiments, the mutated allele is the paternal allele. In some embodiments, the mutated and wild-type allele differ at the PAM sequence motif. In some embodiments, the gRNA is designed such that placement results in cleavage between two identical regions of nucleotides in the mutated allele, which defines the sites of micro-homology. In some embodiments, these regions are about 2 bps to about 3 bps. In some embodiments, these regions are about 3 bps to about 5 bps. In some embodiments, these regions are about 5 bps to about 8 bps. Design of gRNA to meet these parameters is known in the art. gRNA can also be obtained commercially.

The gRNA overlaps the mutation to be specific to the mutant allele. The location of the difference should be as close to the PAM site as possible, ideally within 5, or also within 10 nucleotides from the PAM site. A guide RNA can be tested for its specificity to the SNP by in vitro digestion of PCR products containing the different alleles. The specificity test can also be done in cell lines containing heterozygous for the different SNPs and analysis of indel frequency. The gRNA is also designed to cleave between regions of microhomology that can result in predictable removal of a certain number of nucleotides. The number is determined by the nature of the frame shift mutation. For instance, if one nucleotide is missing due to the mutation, a region of microhomology is sought that is 2, or 5, or 8 bp apart, to result in a novel allele with 1, 2, or 3 amino acids fewer than the wild type allele. Despite this change, this restores the entire rest of the protein and will in many cases be functional. Functionality of the novel protein can be tested in a cell culture or animal model.

In some embodiments, a single gRNA is used.

In some embodiments, the subject is an embryo.

In some embodiments, the guide RNA can be introduced into the embryo as a RNA molecule. The RNA molecule can be transcribed in vitro. Alternatively, the RNA molecule can be chemically synthesized.

In other embodiments, the guide RNA can be introduced into the embryo as a DNA molecule. In such cases, the DNA encoding the guide RNA can be operably linked to promoter control sequence for expression of the guide RNA in the cell or embryo of interest. For example, the RNA coding sequence can be operably linked to a promoter sequence that is recognized by RNA polymerase III (Pol III). Examples of suitable Pol III promoters include, but are not limited to, mammalian U6 or H1 promoters. In some embodiments, the RNA coding sequence is linked to a human U6 promoter. In other exemplary embodiments, the RNA coding sequence is linked to a human H1 promoter.

The DNA molecule encoding the guide RNA can be linear or circular. In some embodiments, the DNA sequence encoding the guide RNA can be part of a vector. Suitable vectors include plasmid vectors, phagemids, cosmids, artificial/mini-chromosomes, transposons, and viral vectors. In some embodiments, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In embodiments in which both the RNA-guided endonuclease and the guide RNA are introduced into the cell as DNA molecules, each can be part of a separate molecule (e.g., one vector containing endonuclease coding sequence and a second vector containing guide RNA coding sequence) or both can be part of the same molecule (e.g., one vector containing coding (and regulatory) sequence for both the endonuclease and the guide RNA).

In some embodiments, the RNA-guided endonuclease and gRNA are introduced into the cell or embryo in the form of a ribonucleoprotein complex comprising the endonuclease complexed to least one gRNA. Preparation of such RNP complexes are known in the art or can be obtained commercially.

In some embodiments, the RNP is introduced into an oocyte at the same time as a sperm cell. This can be accomplished using intracytoplasmic sperm injection (ICSI).

The RNA-targeted endonuclease(s) (or encoding nucleic acid), and the guide RNA(s) (or encoding DNA), can be introduced into an embryo by a variety of means. In some embodiments, the embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3.sup.rd edition, 2001).

In other embodiments, the molecules are introduced into the embryo by microinjection. Typically, the embryo is a fertilized one-cell or two-cell stage embryo.

The method further comprises maintaining the embryo under appropriate conditions such that the guide RNA(s) directs the RNA-guided endonuclease(s) to the targeted site(s) in the allele, and the RNA-guided endonuclease(s) introduce at least one double-stranded break in the allele.

An embryo can be cultured in vitro (e.g., in cell culture). Typically, the embryo is cultured at an appropriate temperature and in appropriate media with the necessary O₂/CO₂ ratio to allow the expression of the RNA endonuclease and guide RNA, if necessary. Suitable non-limiting examples of media include M2, M16, KSOM, BMOC, and HTF media. A skilled artisan will appreciate that culture conditions can and will vary.

In some embodiments, the clinical work flow of using the method herein to correct a frame shift mutation in an embryo would be as follows.
1. Genotyping of the somatic cells of both an egg and sperm donor is performed, typically in a fertility clinic or other clinical setting. The cells are sent to a genotyping facility, which may be in a commercial setting and may be a clinical setting.
   The genotyping is performed to determine: 1. the location of the mutated allele; and 2. the specific mutation on the allele.
2. The next step is to obtain a guide RNA based upon the sequence of the mutated allele. This gRNA can be designed as described herein or such gRNA can be obtained commercially.
   An RNP is then produced using the gRNA and an RNA-guided endonuclease (e.g., Cas9) using methods known in the art. Alternatively, the RNP can be obtained commercially.
3. The RNP is then introduced into the oocyte using ICSI at the same time as the donor sperm. The embryo is then cultured to the blastocyst stage and a tropectoderm biopsy is performed to determine the karyotype. Embryos with a normal genotype are implanted. The blastocyst may be frozen prior to implantation.

In some embodiments, the subject is a fetus, newborn, a child or an adult, and the mutated allele to be corrected is in a somatic cell. The gRNA/RNA guided endonuclease can be delivered to the subject or cell using one or more viruses including recombinant adeno-associated viral (AAV) vectors (e.g., 1, 2, 3, 4, 5, 6, 7, 8, 9, or more AAV vectors). One or more gRNAs (e.g., sgRNAs) can be packaged into single (one) recombinant AAV vector. An RNA-guided endonuclease can be packaged into the same, or alternatively separate recombinant AAV vectors.

In these embodiments, a variety of known viral constructs may be used to deliver the sgRNA(s) and endonucleases to the targeted cells and/or a subject. Nonlimiting examples of such recombinant viruses include recombinant adeno-associated virus (AAV), recombinant adenoviruses, recombinant lentiviruses, recombinant retroviruses, recombinant poxviruses, and other known viruses in the art, as well as plasmids, cosmids, and phages. Options for gene delivery viral constructs are well known.

Additionally, delivery vehicles such as nanoparticle- and lipid-based mRNA or protein delivery systems can be used as an alternative to AAV vectors. Further examples of alternative delivery vehicles include lentiviral vectors, ribonucleoprotein (RNP) complexes, lipid-based delivery system, gene gun, hydrodynamic, electroporation or nucleofection microinjection, and biolistics.

The present methods may utilize adeno-associated virus (AAV) mediated genome engineering. AAV vectors possess a broad host range; transduce both dividing and non-dividing cells in vitro and in vivo and maintain high levels of expression of the transduced genes. Viral particles are heat stable, resistant to solvents, detergents, changes in pH, temperature, and can be concentrated on CsCl gradients. AAV is not associated with any pathogenic event, and transduction with AAV vectors has not been found to induce any lasting negative effects on cell growth or differentiation. In contrast to other vectors, such as lentiviral vectors, AAVs lack integration machinery and have been approved for clinical use (Wirth et al. 2013. Gene 525(2):162-9).

The single-stranded DNA AAV viral vectors have high transduction rates in many different types of cells and tissues. Upon entering the host cells, the AAV genome is converted into double-stranded DNA by host cell DNA polymerase complexes and exist as an episome. In non-dividing host cells, the episomal AAV genome can persist and maintain long-term expression of a therapeutic transgene.

AAV vectors and viral particles of the present disclosure may be employed in various methods and uses. In one embodiment, a method encompasses delivering or transferring a heterologous polynucleotide sequence into a patient or a cell from a patient and includes administering a viral AAV particle, a plurality of AAV viral particles, or a pharmaceutical composition of a AAV viral particle or plurality of AAV viral particles to a patient or a cell of the patient, thereby delivering or transferring a heterologous polynucleotide sequence into the patient or cell of the patient.

The characterization of new AAV serotypes has revealed that they have different patterns of transduction in diverse tissues. AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes.

The term AAV covers all subtypes, serotypes and pseudotypes, and both naturally occurring and recombinant forms, except where required otherwise. Pseudotyped AAV refers to an AAV that contains capsid proteins from one serotype and a viral genome of a second serotype.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

The recombinant AAV containing the desired recombinant DNA can be formulated into a pharmaceutical composition. Such formulation involves the use of a pharmaceutically and/or physiologically acceptable vehicle or carrier.

In certain embodiments, the pharmaceutical composition described above is administered to the subject by direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. The current disclosure also includes systems comprising RNA-guided endonucleases (e.g., Cas9) or DNA encoding the endonuclease, gRNA(s) (or DNA encoding the gRNAs) designed to target the mutated allele, and/or delivery systems of such components such as vectors and RNPs comprising these components.

### CRISPR/Cas and Other Endonucleases

Any suitable nuclease may be used in the present methods and systems. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

One aspect of the present disclosure provides RNA-guided endonucleases. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences.

One example of a RNA guided sequence-specific nuclease system that can be used with the methods and compositions described herein includes the CRISPR system (Wiedenheft, et al. 2012 Nature 482:331-338; Jinek, et al. 2012 Science 337:816-821; Mali, et al. 2013 Science 339:823-826; Cong, et al. 2013. Science 339:819-823). The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

It is appreciated by those skilled in the art that gRNAs can be generated for target specificity to target a specific gene, optionally a gene associated with a disease, disorder, or condition. Thus, in combination with Cas9, the guide RNAs facilitate the target specificity of the CRISPR/Cas9 system. Further aspects such as promoter choice, may provide additional mechanisms of achieving target specificity, e.g., selecting a promoter for the guide RNA encoding polynucleotide that facilitates expression in a particular organ or tissue. Accordingly, the selection of suitable gRNAs for the particular disease, disorder, or condition is contemplated herein. In one embodiment, the gRNA hybridizes to a gene or allele that comprises a single nucleotide polymorphism (SNP).

Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, CasX, Cas12e, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillusferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. In some cases, the dCas9 harbors both D10A and H840A mutations of the amino acid sequence of S. pyogenes Cas9. In some embodiments when a dCas9 has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

The present methods and systems may use CRISPR deletion (CRISPRd). CRISPRd capitalizes on the tendency of DNA repair strategies to default towards NHEJ and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In addition to well characterized CRISPR-Cas system, a new CRISPR enzyme, called Cpf1 (Cas protein 1 of PreFran subtype) may be used in the present methods and systems (Zetsche et al. 2015. Cell). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the nuclease is a transcription activator-like effector nuclease (TALEN). TALENs contains a TAL effector domain that binds to a specific nucleotide sequence and an endonuclease domain that catalyzes a double strand break at the target site (PCT Patent Publication No. WO2011072246; Miller et al., 2011 Nat. Biotechnol. 29:143-148; Cermak et al., 2011 Nucleic Acid Res. 39:e82). Sequence-specific endonucleases may be modular in nature, and DNA binding specificity is obtained by arranging one or more modules. Bibikova et al., 2001 Mol. Cell. Biol. 21:289-297; Boch et al., 2009 Science 326:1509-1512.

ZFNs can contain two or more (e.g., 2-8, 3-6, 6-8, or more) sequence-specific DNA binding domains (e.g., zinc finger domains) fused to an effector endonuclease domain (e.g., the FokI endonuclease). Porteus et al., 2005 Nat. Biotechnol. 23:967-973; Kim et al., 2007 Proceedings of the National Academy of Sciences of USA, 93:1156-1160; U.S. Pat. No. 6,824,978; PCT Publication Nos. WO1995/09233 and WO1994018313.

In one embodiment, the nuclease is a site-specific nuclease of the group or selected from the group consisting of omega, zinc finger, TALEN, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. 2002 Nucleic Acids Research 30:3870-3879. Combinatorial assembly is a method where protein subunits form different enzymes can be associated or fused. Arnould et al. 2006 Journal of Molecular Biology 355:443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Guide RNA(s) used in the methods of the present disclosure can be designed so that they direct binding of the Cas-gRNA complexes to pre-determined cleavage sites in a genome. In one embodiment, the cleavage sites may be chosen so as to release a fragment or sequence that contains a region of a frame shift mutation. In further embodiment, the cleavage sites may be chosen so as to release a fragment or sequence that contains an extra chromosome.

For Cas family enzyme (such as Cas9) to successfully bind to DNA, the target sequence in the genomic DNA can be complementary to the gRNA sequence and may be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

gRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 9192, 93, 94, 95, 96, 97, 98, 99, or 100 nucleotides in length, or longer). In one embodiment, gRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed (See Prykhozhij et al. 2015 PLoS ONE 10(3); Zhu et al. 2014 PLoS ONE 9(9); Xiao et al. 2014 Bioinformatics. January 21 (2014)); Heigwer et al. 2014 Nat Methods 11(2):122-123). Methods and tools for guide RNA design are discussed by Zhu 2015 Frontiers in Biology 10(4):289-296, which is incorporated by reference herein. Additionally, there is a publicly available software tool that can be used to facilitate the design of gRNA(s) (www.genscript.com/gRNA-design-tool) and https://www.idtdna.com/pages/products/crispr-genome-editing/alt-r-crispr-cas9-system).

### Single-Nucleotide Polymorphism (SNP)

A single-nucleotide polymorphism, often abbreviated to SNP, is a substitution of a single nucleotide that occurs at a specific position in the genome, where each variation is present to some appreciable degree within a population (e.g., >1%). In one embodiment, at a specific base position in the human genome, the A nucleotide may appear in most individuals, but in a minority of individuals, the position is occupied by a G. This means that there is a SNP at this specific position, and the two possible nucleotide variations—A or G—are said to be alleles for this position. The term SNP refers to a difference of one base at the same relative site when two alleles are aligned and compared; herein, the term is also used in some contexts to mean a single base change.

A single-nucleotide variant (SNV) is a variation in a single nucleotide without any limitations of frequency and may arise in somatic cells.

The variant of the SNP may be a G variant, a C variant, an A variant, or a T variant.

The one or more SNPs may be in one or more coding regions, one or more non-coding regions, one or more intergenic regions (regions between genes), or combinations of one or more coding regions, and/or one or more non-coding regions, and/or one or more intergenic regions. The one or more SNPs may be in one or more introns, one or more exons, a combination of one or more introns and one or more exons. SNPs within a coding region may or may not change the amino acid sequence of the protein that is produced. SNPs may be synonymous SNPs or nonsynonymous SNPs. Synonymous SNPs do not affect the protein sequence, while nonsynonymous SNPs change the amino acid sequence of protein. The nonsynonymous SNPs may be missense SNPs or nonsense SNPs. SNPs that are not in protein-coding regions may affect gene splicing, transcription factor binding, messenger RNA degradation, and/or the sequence of noncoding RNA. Gene expression affected by this type of SNP may be upstream or downstream from the gene.

Genotyping of polymorphic variants can be carried out using any suitable methodology known in the art. Techniques which may be used for genotyping single nucleotide polymorphisms (SNPs) include DNA sequencing; capillary electrophoresis; ligation detection reaction (Day et al., 1995. Genomics 29:152-62); mass spectrometry, such as matrix-assisted laser desorption/ionization time-of-flight mass spectrometry (MALDI-TOF-MS); single-strand conformation polymorphism (SSCP); single-base extension; electrochemical analysis; denaturating HPLC and gel electrophoresis; restriction fragment length polymorphism; hybridization analysis; single nucleotide primer extension and DNA chips or microarrays (see review by Schafer et al., 1998 Nature Biotechnology, 16:33-39). The use of DNA chips or microarrays may enable simultaneous genotyping at many different polymorphic loci in a single individual or the simultaneous genotyping of a single polymorphic locus in multiple individuals. SNPs may also be scored by DNA sequencing.

In addition to the above, SNPs may be scored using PCR-based techniques, such as PCR-SSP using allele-specific primers (Bunce et al., 1995 Tissue Antigens, 50:23-31). This method generally involves performing DNA amplification reactions using genomic DNA as the template and two different primer pairs, the first primer pair comprising an allele-specific primer which under appropriate conditions is capable of hybridizing selectively to the wild type allele and a non allele-specific primer which binds to a complementary sequence elsewhere within the gene in question, the second primer pair comprising an allele-specific primer which under appropriate conditions is capable of hybridizing selectively to the variant allele and the same non allele-specific primer. Further suitable techniques for scoring SNPs include PCR ELISA and denaturing high performance liquid chromatography (DHPLC).

If the SNP results in the abolition or creation of a restriction site, genotyping can be carried out by performing PCR using non-allele specific primers spanning the polymorphic site and digesting the resultant PCR product using the appropriate restriction enzyme (also known as PCR-RFLP). Restriction fragment length polymorphisms, including those resulting from the presence of a single nucleotide polymorphism, may be scored by digesting genomic DNA with an appropriate enzyme then performing a Southern blot using a labelled probe corresponding to the polymorphic region (Molecular Cloning: A Laboratory Manual, Sambrook, Fritsch and Maniatis, Cold Spring Harbor Laboratory, Cold Spring Harbor, N.Y.).

“Genotyping” of any given polymorphic variant may comprise screening for the presence or absence in the genome of the subject of both the normal or wild type allele and the variant or mutant allele, or may comprise screening for the presence or absence of either individual allele, it generally being possible to draw conclusions about the genotype of an individual at a polymorphic locus having two alternative allelic forms just by screening for one or other of the specific alleles.

It is within the scope of the present disclosure to perform genotyping of polymorphisms or polymorphic variants within multiple genes. Such a panel screen of multiple genes may be used to simultaneously analyze multiple polymorphisms in the same subject. In one embodiment, genotyping of multiple polymorphisms in a single subject sample may be carried out simultaneously, for example with the use of a microarray or gene chip.

“Multiple” should be taken to mean two or more, three or more, four or more, five or more, six or more etc.

Genotyping may be carried out in vitro, and can be performed on an isolated sample containing genomic DNA prepared from a suitable sample obtained from the subject under test. For example, genomic DNA is prepared from a sample of whole blood or tissue, or any suitable sample as described herein, according to standard procedures which are well known in the art. If genomic sequence data for the individual under test in the region containing the SNP is available, for example in a genomic sequence database as a result of a prior genomic sequencing exercise, then genotyping of the SNP may be accomplished by searching the available sequence data.

In the case of genetic variants which have a detectable effect on the mRNA transcripts transcribed from a given gene, for example variants which cause altered splicing or which affect transcript termination or which affect the level or mRNA expression, then as an alternative to detecting the presence of the variant at the genomic DNA level, the presence of the variant may be inferred by evaluating the mRNA expression pattern using any suitable technique. Similarly, in the case of genetic variants which have a detectable effect on the protein products encoded by a gene, for example variants which cause a change in primary amino acid sequence, structure or properties of the encoded protein, the presence of the variant may be inferred by evaluating the sequence, structure or properties of the protein using any convenient technique.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-9

### Gamete Donation

Oocyte donors were recruited from subjects participating in the Columbia Fertility oocyte donor program and were provided the option to donate for research instead of for reproductive purpose. Oocyte donors underwent controlled ovarian stimulation and oocyte retrieval as in routine clinical practice and as previously described (Zakarin et al., 2018). Oocyte donors also provided a vial of blood (3-5 ml) for isolation of genomic DNA and a skin biopsy. 67 oocytes from a total of 8 different oocyte donors were used, ages from 27-31 years. Oocytes were cryopreserved using Cryotech vitrification kit until genotyping of the mutation site and flanking SNPs was completed. Oocytes were thawed using the Cryotech warming kit. The sperm donor provided material via Male-FactorPak collection kit (Apex Medical Technologies MFP-130), which was cryopreserved using washing medium from MidAtlantic Diagnostics (ART-1005) and TYB freezing media from Irvine Scientific (90128). All gamete donors provided signed informed consent. All human subjects research was reviewed and approved by the Columbia University Embryonic Stem Cell Committee and the Institutional Review Board.

### RNP Preparation

Guide RNA 5′-GUGUGUCUUUCUUCUGUACUGGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 1) was obtained from Synthego, with the target RNA underlined. EnGen Cas9 NLS, S. pyogenes was obtained from NEB (M0646T). For ribonucleoprotein (RNP) preparation 3.125 μL of 20 μM Cas9 and 0.776 μL of 100 μM sgRNA was combined and incubated at room temperature for 10 minutes, followed by addition of 46 μl injection buffer consisting of 5 mM Tris-HCl, 0.1 mM EDTA, pH 7.8.

### Oocyte Manipulations

All manipulations were performed in an inverted Olympus IX71 microscope using Narishige micromanipulators on a stage heated to 37° C. using Global Total w. HEPES (LifeGlobal LGTH-050). Oocyte enucleation for androgenesis was performed as previously described (Yamada et al. 2014; Sagi et al. 2019). Briefly, oocytes were enucleated in 5 Dg/ml cytochalasinB (Sigma-Aldrich C2743), the zona pellucida was opened using a zona laser (Hamilton Thorne) set at 100% for 300 μs. The spindle was visualized using microtubule birefringence and removed using a 20 μm inner diameter Piezo micropipette (Humagen). Intracytoplasmic sperm injection was identical for both nucleated and enucleated metaphaseII oocytes. Sperm was thawed to room temperature for 10 minutes and transferred to a 15 mL conical tube. Quinn's Sperm Washing Medium (Origio) was added dropwise to a final volume of 6 mL. The tube was then centrifuged at 300×g for 15 minutes. Supernatant was removed and an additional wash was performed. Upon removal of supernatant from second wash, pellet was suspended in wash media and analyzed for viability.

Manipulation dishes consisted of a droplet with 10% PVP, a 10-20 μl droplet with RNP in injection buffer, and a droplet of Global Total w. HEPES. Sperm was mixed with 10% PVP (Vitrolife), and individual sperm was immobilized by pressing the sperm tail with the ICSI micropipette (Humagen), picked up and transitioned through the RNP droplet before injection. After all manipulations, cells were cultures in Global total (LifeGlobal) in an incubator at 37° C. and 5% C02. Pronucleus formation was confirmed on day 1 after ICSI. 2-cell injections were performed at least 3 hours after cleavage to avoid lysis, between 30-35 h post ICSI. The tip of an injection needle was nicked and small amounts of the Cas9RNP was injected manually using a Narishige micromanipulator.

### Genome Amplification and Genotyping

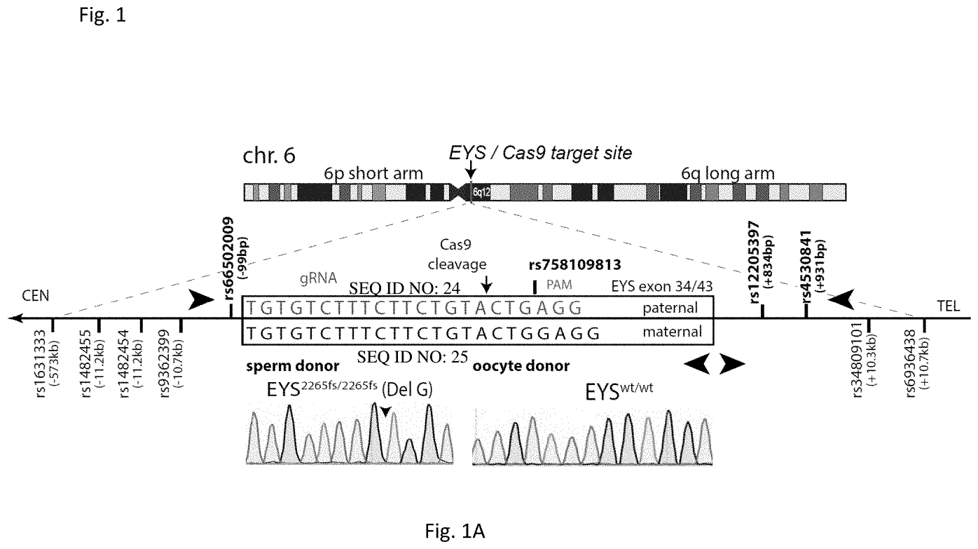

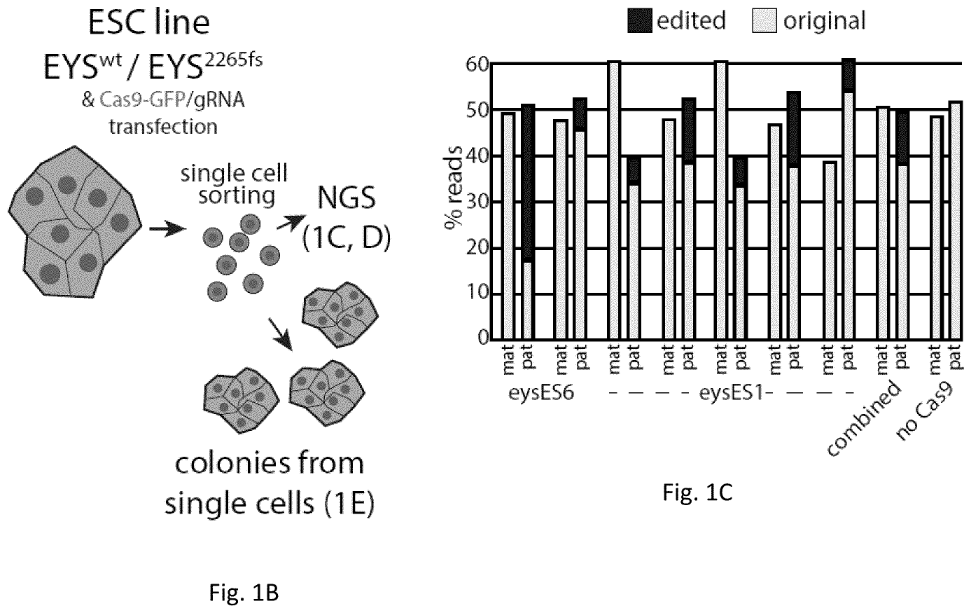

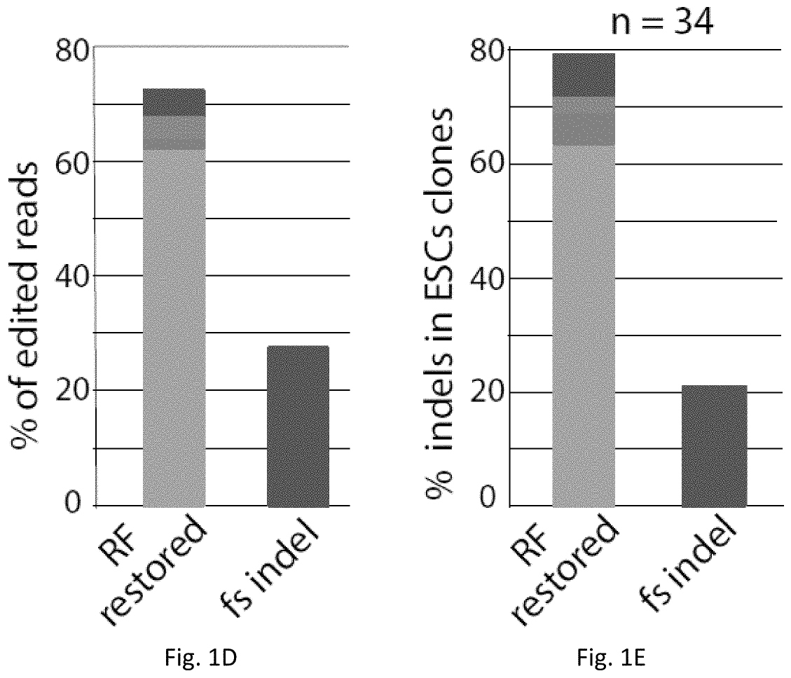

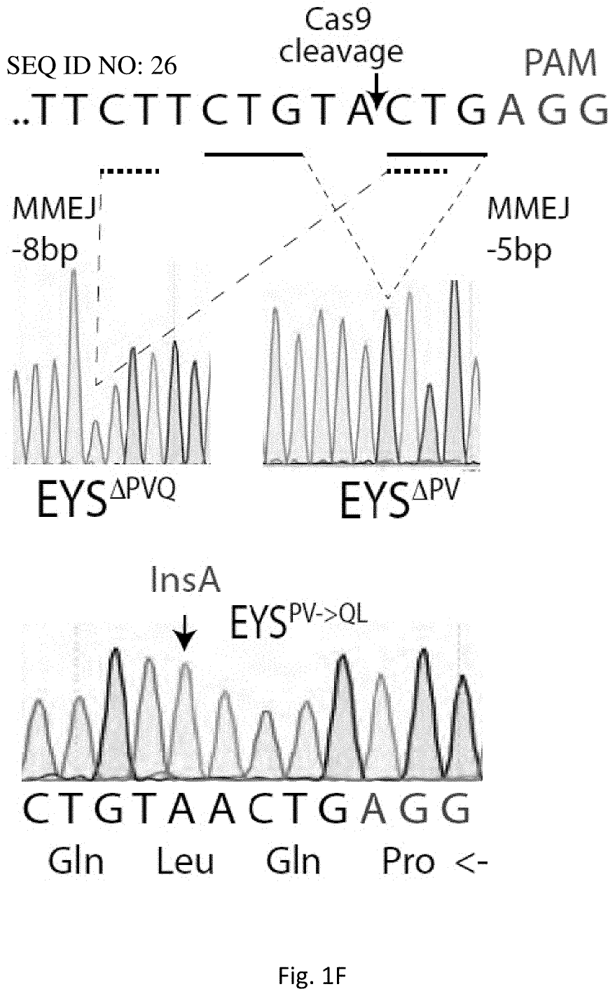

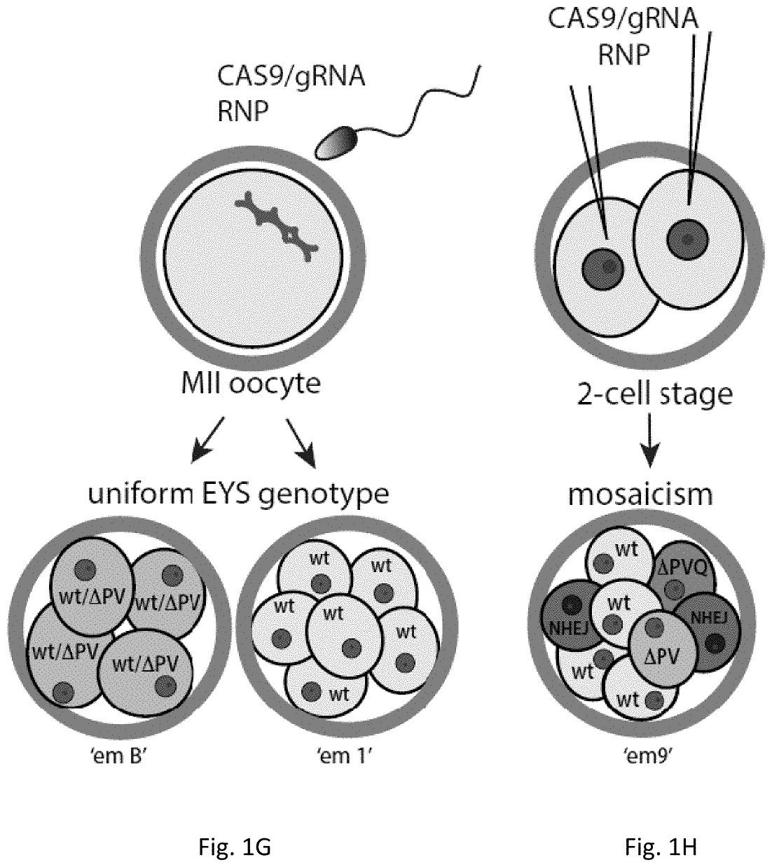

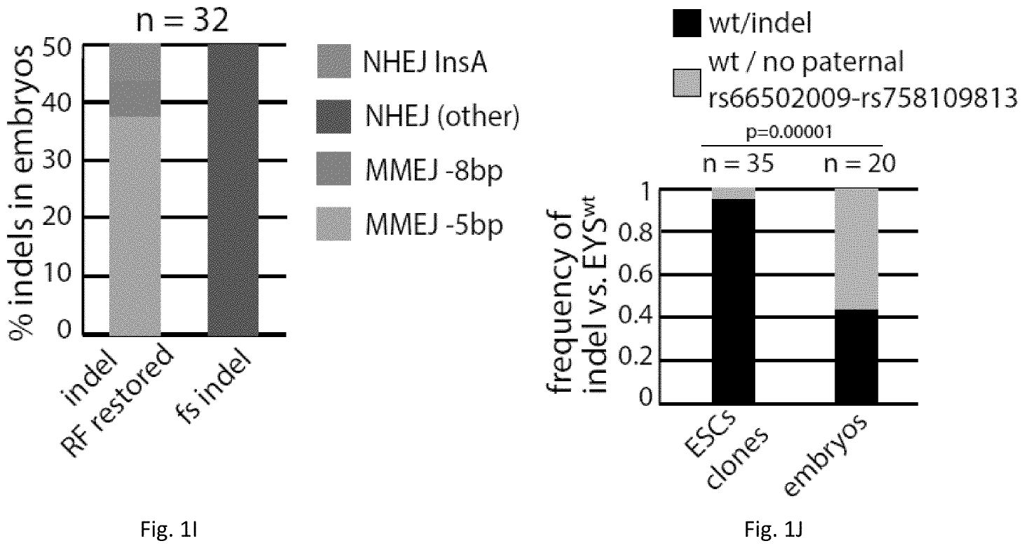

Zygotes were collected at 20 h post ICSI, and single blastomeres on day 3 to day 4. Trophectoderm biopsies were obtained on day6 of development using 300 ms laser pulses to separate trophectoderm from the inner cell mass. Single zygote nuclei were extracted from zygotes in the presence of 10 mg/ml CytochalasinB and 1 mg/ml nocodazole at 20 h post ICSI. All samples were placed in single tubes with 2 μl PBS. Amplification was performed using either Illustra GenomePhi V2 DNA amplification kit, or REPLI-g single cell kit (Qiagen Cat #150345) according to manufacturer's instructions. Genotyping was performed using primers for amplification and sequencing. PCR was performed using AmpliTaq Gold (ThermoFisher Cat. #4398886). TOPO-TA cloning (Thermo Fisher Cat #450640) was done for heterozygous edits and 5-10 colonies sequenced to identify the two edits individually (e.g., FIG. 1F).

For on target deep sequencing, primers were designed to amplify the specific region of the EYS gene. Once amplified, the PCR product was purified via sodium acetate precipitation. After purification samples were sent out for Illumina Amplicon Next Generation sequencing (Genewiz). Results were given in the format of raw reads and fastq files and an analysis of read frequency was conducted. Each sample yielded >50,000 reads. Sequences with a read count that exceeded 0.2% of the total reads (>100 reads) were considered significant. Those that had less than 100 reads were considered insignificant due to the possibility that these were due to PCR artifacts or sequencing errors. Base changes were analyzed at the region of Cas9 cutting, and paternal and maternal alleles were called based on rs66502009, and rs758109813.

Cas9 off-target analysis was performed by identifying potential off-target sites using Cas-OFF finder online tool (http://www.rgenome.net/cas-offinder/), selecting SpCas9 of Streptococcus pyogenes. 32 potential off-target sites were predicted across 14 chromosomes. Those with 3 or fewer mismatches were selected for analysis. PCR primers for off-target analysis are indicated in Table S7.

EYS Mutation qPCR

A TaqMan assay (C_397916532_10) was used to obtain allelic discrimination results for the EYS mutation (rs758109813; NG_023443.2:g.1713111del) using a QuantStudio 3 instrument and following the manufacturer's recommendations (ThermoFisher).

### Genome-Wide SNP Array

Embryo biopsies were amplified at Columbia University using either RepliG or GenomePhi as described above, or at Genomic Prediction Clinical Laboratory using ePGT amplification (Treff et al., 2019). Amplified DNA was processed according to the manufacturer's recommendations for Axiom GeneTitan UKBB SNP arrays (ThermoFisher). Copy number and genotyping analysis was performed using gSUITE software (Treff et al., 2019). Parental origin of copy number changes was determined by genotype comparison of embryonic and parental SNPs.

For copy number analysis, raw intensities from Affymetrix Axiom array are first processed according to the method described (Mayrhofer et al., 2016). After normalizing with a panel of normal males, the copy number is then calculated for each probeset. Normalized intensity is displayed. Mapping of endogenous fragile sites was done through visual evaluation of loss of heterozygosity. Break points were mapped to chromosomal bands by visual analysis of SNP array chromosome plots including analysis of both copy number signal and heterozygosity calls. The accuracy of mapping is between 100-500 kb. Evaluation whether a segmental error involved a common fragile site was performed by comparison of break sites to the location of common fragile sites according to (Mrasek et al., 2010).

### Derivation and Culture of ES Cells

Stem cells were derived after trophectoderm biopsy and plating for the inner cell mass as previously described (Yamada et al., 2014). Briefly, mural trophectoderm was ablated using laser-assisted pulses 400 μs, 100% intensity (Hamilton Thorne) (Chen et al., 2009). This method spares polar trophectoderm, which usually results in trophectoderm growth which is then ablated with additional pulses. Two stem cell lines with the parental genotypes wt/EYS^{2265fs}, eysES6 and eysES1, were obtained and used for experiments to determine mechanisms of repair after Cas9 mediated cleavage of the eys mutant allele. Stem cells were cultured with StemFlex (Thermo Fisher A3349401) media on Geltrex (Thermo Fisher A1413302). Upon reaching 70% confluency, cultures were passaged at a ratio of 1:10, or cryopreserved in a solution of freezing media containing 40% FBS (Company) and 10% DMSO (Sigma). Passaging was performed by TrypLE dissociation to small clusters of cells and plated in media containing Rock inhibitor Y-27632 (Selleckchem S1049) was added to media and removed within 24-48 hours. For later passage cells (>passage 10), Rock inhibitor was omitted.

DSB repair in EYS^{wt}/EYS^{2265fs} s ES Cells

For CRISPR-Cas9 gene editing, cultures of embryonic stem cell lines eysES1 and eysES6, both of which have the EYS^{wt}/EYS^{2265fs} genotype and are heterozygous at rs66502009 were dissociated to single cells, and cells were nucleofected with Cas9-GFP (Addgene #44719), a gBlock (IDT) expression vector, using the Lonza Kit (VVPH-5012) and Amaxa Nucleofector. 1 million cells at 50% confluency at time of use were nucleofected per reaction using program A-023. GFP positive cells were sorted using BD SORP FACSAria cell sorters 48 h post nucleofection at the Columbia Stem Cell Initiative Flow Cytometry Core.

For NGS analysis, 2000-5,000 sorted cells were harvested for genome amplification using identical methods as used for embryo blastomeres. Whole genome amplification was performed using REPLI-G (Qiagen). PCR was then performed using primers flanking EYS^{2265fs} and rs66502009 and products were analyzed for gene editing using AmpliconEZ service of Genewiz. Read frequency analysis was used to quantify molecularly distinct edits. Paternal and maternal alleles were distinguished based on EYS2265fs and rs66502009.

For clonal analysis, two independent experiments with eysES1 and eysES6 were performed. Single Cas9-GFP positive sorted cells were plated in individual wells of 96-well plates onto Geltrex with StemFlex media with Rock inhibitor Y-27632 (Selleckchem). After 5-12 days, colonies were picked and harvested for DNA isolation and analysis. DNA collection from cultured cells was performed using QuickExtract (Lucigen QE09050). PCR and sequencing was performed using primers flanking the gRNA at EYS2265fs as well as rs66502009. The paternal and maternal alleles were identified using rs66502009 and EYS2265fs, and Cas9-induced modifications were called through visual analysis and using ICE (Synthego). Sanger profiles from all edited colonies contained at most two molecularly different alleles, and equal signal intensity from the edited paternal allele and from the non-edited maternal allele, confirming that they were individual events originating from single cells.

### Example 2—Allele-Specific Editing of the EYS Gene in Stem Cells

A sperm donor with a homozygous G deletion mutation (rs758109813) in the gene encoding EYS associated with retinitis pigmentosa was recruited (Abd El-Aziz et al., 2008). This mutation results in the frame-shift p.Pro2265Glnfs*46 in exon 34 (referred to as EYS^{2265fs}) The EYS gene is located on chromosome 6, at 6q12, near the centromere of the long arm. A guide RNA (gRNA) was designed to specifically target the mutant but not the wild type allele, which differs at the PAM sequence motif. Pairing of the gRNA with the maternal allele at the 5′-AGG PAM site would require a 1 bp bulge, for which there is low tolerance at this location of the gRNA (Lin et al., 2014). The Cas9 cleavage site occurs proximal to EYS2^{265fs}, such that small indels will preserve the original SNP, which can distinguish modified alleles of paternal or maternal origin. The mutation is flanked by common SNPs, rs66502009, centromeric to the mutation, and rs12205397 and rs4530841 telomeric to the mutation, which are amplified within a single ˜1 kb PCR product, as well as by SNPs at greater distance. Thus, oocytes from donors with homozygous SNPs that differ from the sperm donor allow the identification of maternal and paternal chromosomes in the embryo, and the evaluation of novel combinations of maternal and paternal alleles. (FIG. 1A, Table 2).

First an embryonic stem cell line was derived through fertilization to determine the specificity of the gRNA for the mutant allele (FIG. 1B). Heterozygous stem cells for both the mutation (wt/EYS^{2265fs}) and rs66502009 were transfected with Cas9-GFP and gRNA vectors to target the EYS^{2265fs} allele, and GFP positive cells were harvested by flow cytometry 48 hours post transfection. Using PCR and on-target next-generation sequencing (NGS) of seven independent biological samples scoring for SNP rs66502009 and EYS^{2265fs}, it was found 51% (199,780 of 392,672) were unmodified reads of the maternal EYS^{wt} allele, while 49% were of paternal origin, of which 11% (range 5-33%, n=7) were edited to contain small indels on reads containing both the EYS2265fs and the rs66502009 paternal allele (FIG. 1C). This demonstrates the specificity of the gRNA for the paternal allele, resulting in efficient modification at the paternal mutation site EYS^{2265fs}.

### Example 3—End Joining Restores the EYS Reading Frame by Inducing Predictable Indels in ESCs and Embryos

Analysis of the types of edits in NGS reads showed that the most frequent event (63.5% of edited reads) was the deletion of 5 bp on the paternal allele (FIG. 1D), resulting in the restoration of the reading frame. Deletion of 8 bp (1%) and insertion of 1 bp (3.5%), both of which restore the reading frame, as well as reading frame restorations from other indels (4.5%) were also observed. Another 27.5% of reads carried indels without reading frame restoration. In addition to analysis of NGS reads, single cells were plated for colony formation representing biologically independent editing events. In 245 clones grown from single cells, 35 edited clones were identified (14.3%), 34 of which had indels in the paternal allele, while one had no detectable paternal allele at either rs66502009 or EYS²²⁶⁵. Of the 34 clones with indels, the reading frame was restored in 27 (79.4%), primarily through a recurrent 5 bp deletion (FIG. 1E). The placement of the guide RNA resulted in cleavage between two identical regions of either 3 bp or 2 bp, defining sites of micro-homology (FIG. 1F). Microhomology-mediated end joining (MMEJ) is an efficient repair pathway that uses one to a dozen base pairs of homology (Sfeir and Symington, 2015). MMEJ at the EYS locus results in the deletion of either 5 bp or 8 bp, thereby restoring the reading frame, and generating two different novel alleles with deletions of either two amino acids (p.P2265-V2266) (EYS^{DPV}) or three amino acids (p.Pro2265_Gln2267del) (EYS^{DPVQ}). The deletion EYS^{DPV} was by far the most common single repair product in ESCs. No mitotic recombination between rs758109813 and rs66502009, which would be indicative of interhomolog repair, was observed in the 34 edited clones.

### Example 4—EYS Reading Frame Restoration in Embryos

To determine editing outcomes in human embryos, EYS^{2265fs} mutant sperm was injected into the cytoplasm (ICSI) together with a ribonucleoprotein (RNP) complex of Cas9 nuclease and the gRNA targeting EYS^{2265fs} (FIG. 1G). Alternatively, Cas9/RNP was injected after fertilization at the 2-cell stage (FIG. 1H). In both types of injections, embryos were biopsied for genotyping at the cleavage or the blastocyst stage. While injection at the two-cell stage invariably resulted in mosaic embryos (n=13) with up to three genotypes (Table 2), injection at the MII stage resulted in embryos (n=7) that appeared uniform, for either an indel (n=3), or only the EYS^{wt} allele (n=4).

Combining both data from MII injections as well as 2-cell stage injections, it was found that a total of 32 end joining events were independent, because they occurred in different embryos, or differed molecularly within the same embryo (FIG. 1I, Table 2). 14 of these were MMEJ events, 12 of which resulted in a 5 bp deletion and 2 in an 8 bp deletion. Furthermore, of a total of 18 independent NHEJ events, 2 restored the reading frame through insertion of an A nucleotide, resulting in a transition to (p.Pro2265_Val2266insGlnLeu) EYS^{PV>QL} (FIG. 1F). Importantly, in one embryo derived from an MII injection, all blastomeres (4/4) showed uniform reading frame restoration, demonstrating nonmosaic editing through MMEJ (Table 2), The reproducible and frequent generation of the EYS^{DPV} allele in both embryos and stem cells shows that MMEJ can efficiently be used for the restoration of the reading frame at EYS^{2265fs}.

Though the frequency of different end joining events in pluripotent stem cells and embryos was comparable, there was one significant difference in editing outcomes: considering both MII and 2-cell stage injections, 17 of 20 embryos contained cells with only an EYS^{wt} allele and the flanking maternal rs66502009 allele, representing at least 17 independent events, while 12 embryos contained cells with indels, 9 embryos contained both types of cells. Therefore, the loss of the paternal allele was as or more common than a heterozygous indel in embryos (FIG. 1J). Only a single event (2.8%) was seen in Cas9 treated ESC clones. These events could represent unrepaired paternal alleles, undetectably modified paternal alleles (Adikusuma et al., 2018; Kosicki et al., 2018), or interhomolog repair as previously suggested (Ma et al., 2017). These results indicate cell type differences in double-strand break repair and/or cell survival after a chromosome break.

### Example 5—Undetectable Paternal Alleles within the First Cell Cycle after Cas9 RNP Injection

To better understand the origin of EYS^{wt} genotypes, events in the first cell cycle after MII injection of sperm and Cas9 RNP in either androgenetic zygotes with only a paternal genome, or zygotes with or without isolation of the two pronuclei (2PN) were analyzed. These approaches also lead to a more conclusive determination of mosaicism, which is more reliably achieved when there is just a single cell and genome.

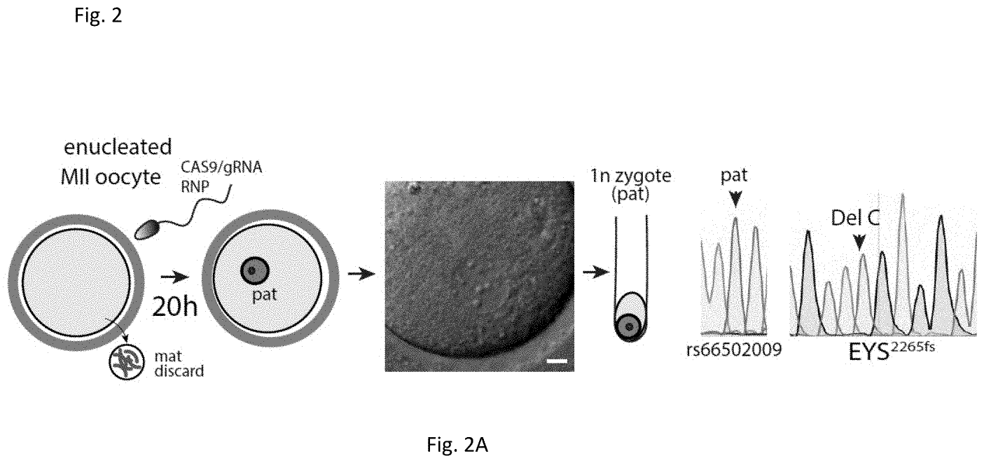

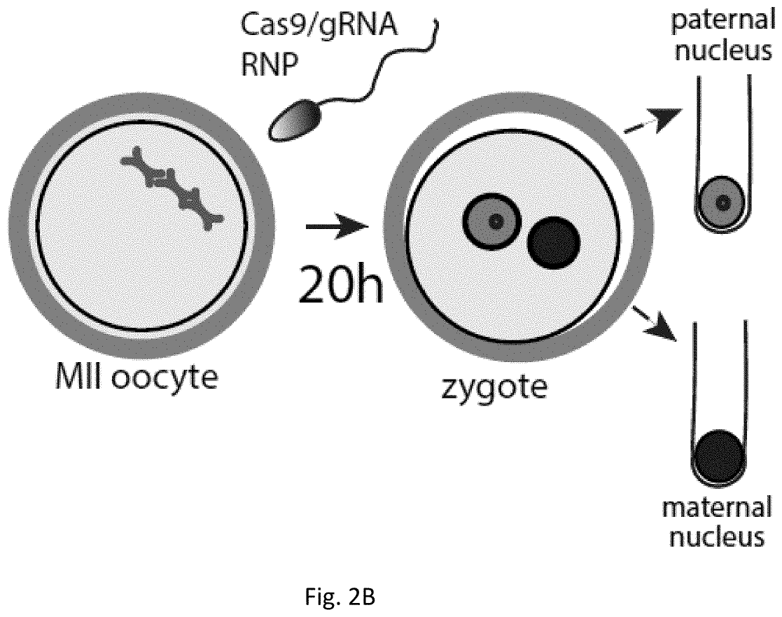

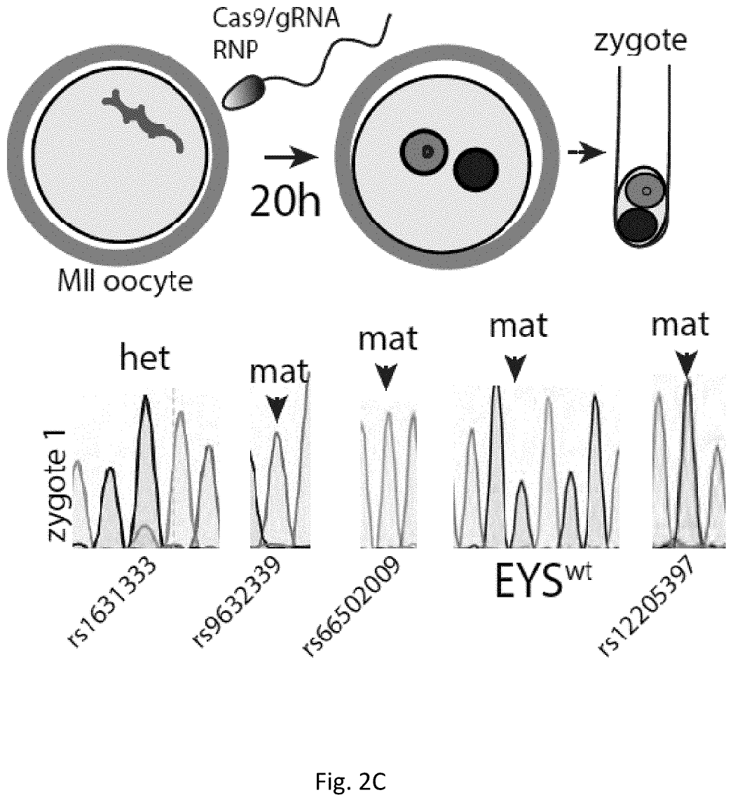

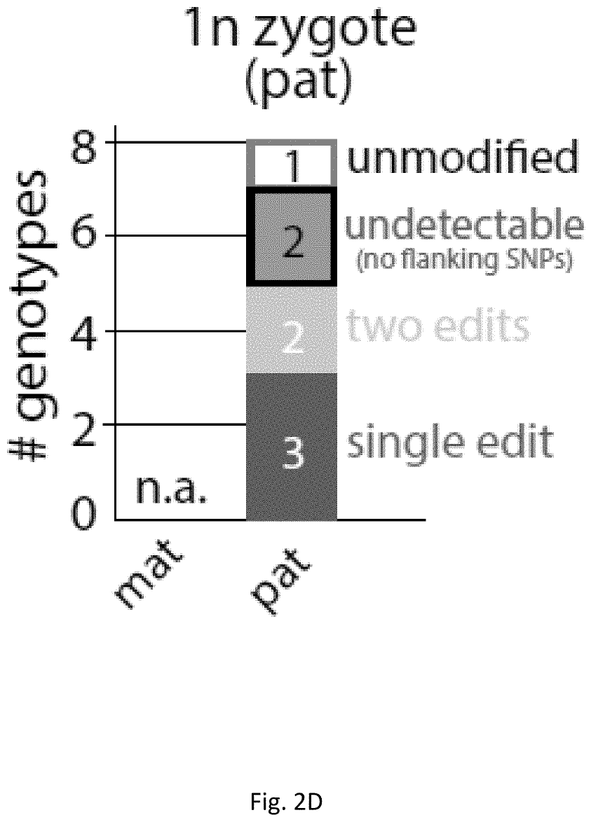

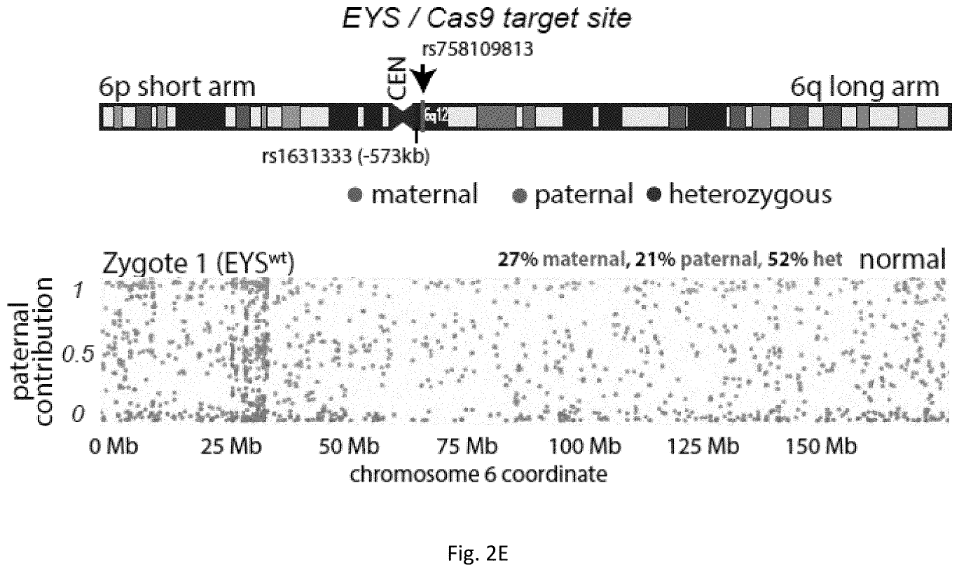

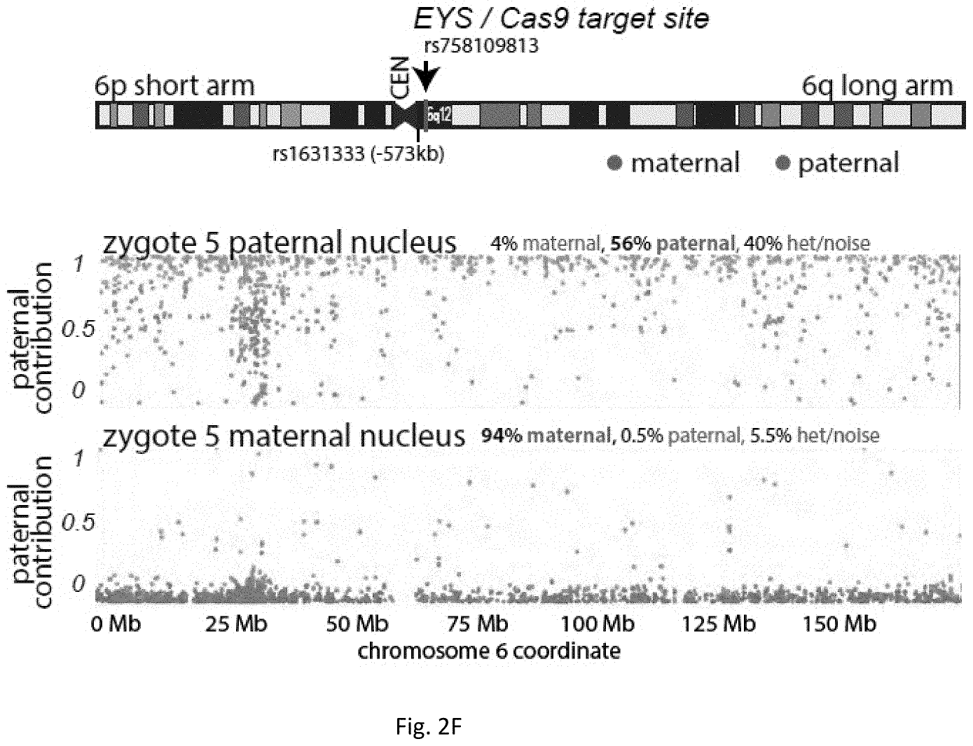

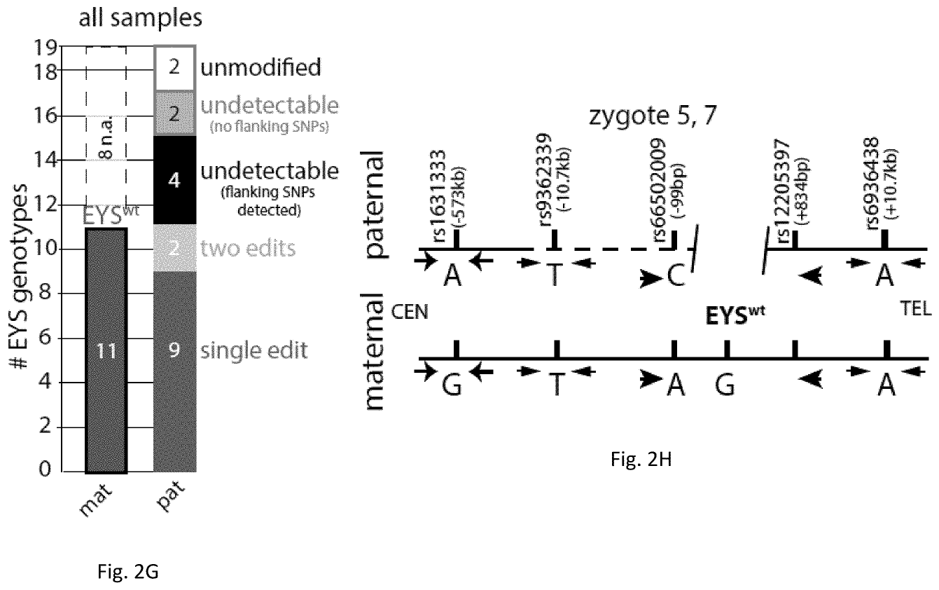

To evaluate the timing of Cas9 cleavage and repair, it was first determined which repair products were present in the absence of a maternal genome using androgenesis. The maternal genome was removed and the EYS^{2265fs} mutant sperm was injected together with a ribonucleoprotein (RNP) complex of Cas9 nuclease and a guide RNA (gRNA) targeting the mutation site (FIG. 2A). At 20 hours post sperm injection, zygotes with a single pronucleus were harvested for analysis. Of 8 androgenetic zygotes from two different oocyte donors (5 and 3 oocytes per donor), 5 were modified, 3 of which were homozygous for a small insertion or deletion, while 2 were heterozygous for two different modified alleles (FIG. 2A, Table 2). In addition, one was unmodified, and two failed to genotype (FIG. 2D).

To determine whether the maternal genome provided a repair template for the paternal genome in zygotes, ICSI with Cas9 RNP was performed next in nucleated oocytes to generate zygotes with both genomes. To avoid the possibility of allele-dropout due to unequal amplification of alleles, single paternal and maternal nuclei from zygotes at 20 h post ICSI were isolated and amplified them separately (FIG. 2B). Of the 8 paternal nuclei, 5 contained an indel on the paternal allele, while one was unmodified, and two failed to genotype at the mutation site but not at flanking SNPs 10 kb away (FIG. 2B, Table 2). All edited genomes showed only a single modification, suggesting that cutting and end joining occurred before replication of EYS. No zygote contained a paternal genome with a wild type allele and thus there was no evidence for repair from the maternal genome among these 8 zygotes.

To determine the accuracy of genotyping in zygotes with both alleles and without mechanical separation into separate tubes, single cells from embryos (n=3) and single ES cells (n=4) containing both genomes without exposure to Cas9 RNP were amplified first. All cells showed heterozygosity at the mutation site as well as at the flanking SNP rs66502009, demonstrating that both alleles were reliably detected (Table 2). Next fertilized zygotes at 20 h post fertilization and Cas9 injection were analyzed (FIG. 2C). Of three fertilized oocytes, one zygote showed an edited paternal allele, while two showed only maternal alleles at the mutation site as well as at flanking SNPs on either side (FIG. 2C, Table 2). For one of the zygotes, a SNP 573 kb away could be determined and was found heterozygous for the paternal and maternal alleles (FIG. 2C). To account for the possibility of allele dropout in Sanger sequencing, on-target NGS of both ‘wild-type’ zygotes was performed, which showed no significant reads of either the paternal mutation or the flanking paternal SNP at rs66502009 (Table 2).

Of a total of 19 zygotes analyzed at the 1-cell stage (20 h post ICSI and Cas9 RNP), 11 of the paternal genomes were detectably modified by end joining events during the first cell cycle (FIG. 2G, Table 2). Editing was predominantly homozygous (9 out of 11 events) and thus nonmosaic zygotes, whereas 2 of the androgenetic embryos were mosaic. Though these mosaics were seen in androgenetic zygotes, they show that MII injection results predominantly, but not exclusively, in uniform editing.

The remaining 6 embryos had no detectable paternal allele at rs758109813. These zygotes could indicate the possibility of a modified paternal allele that cannot be amplified using primers flanking the Cas9 cut site. To determine the cause of paternal allelic loss, flanking SNPs were amplified and genotyped. Neither of two androgenotes showed amplification of flanking SNPs 10 kb distal and proximal to the Cas9 cleavage site and might hence instead be due to amplification failure or extensive resection of the DSB. The two zygotes with only an EYS^{wt} genotype at rs758109813 as well as the two paternal nuclei extracted from 2PN zygotes but without an EYS on target genotype showed amplification at flanking SNPs 10 kb away from the Cas9 cut site and were hence successfully amplified (Table 2).

To more comprehensively analyze chromosome content, genome-wide SNP array analysis was performed. First, genomic DNA from the sperm and oocyte donors was analyzed to identify homozygous donor-specific alleles. Genomic DNA of an egg donor and genomic DNA of the semen donor showed homozygous alleles of maternal and paternal origin along chromosome 6 (results not shown). Only SNPs which were homozygous within the donors but different between the donors were subsequently used for analysis. Results not shown.

SNP array analysis of zygotes after Cas9 RNP injection at fertilization was performed. Zygote 1 containing an EYS^{wt} genotype was successfully analyzed using SNP arrays and signal from paternal and maternal alleles was seen all along chromosome 6 on both the p and q arms (FIG. 2E). 8 paternal and 7 maternal nuclei isolated from eight 2PN zygotes were also analyzed (FIG. 2F). Isolated individual nuclei uniformly showed either paternal or maternal origin, respectively, of chromosome 6 and other autosomes (n=326 chromosomes) (FIG. 2F). No exchange of entire chromosomes or chromosome segments of paternal and maternal origin was observed, neither for chromosome 6 nor on any other autosomes. Therefore, paternal and maternal genomes remained physically separate during the first interphase, regardless of the presence of a DSB. The lack of a paternal genotype at EYS rs758109813 in two individual nuclei and in zygote 1 was not due to loss of the entire paternal chromosome or of a chromosome segment. Importantly, maternal alleles were detected in all (11/11) zygotes, suggesting that the loss of the paternal allele is primarily biological, though technical causes cannot formally be excluded for two androgenetic zygotes without amplification of neighboring alleles.

### Example 6—Loss of Paternal Alleles in Embryos but not Derived Stem Cell Lines

The loss of the paternal EYS allele from 6 zygotes may be due to an unrepaired DSB, which prevents amplification by PCR primers flanking the cut site (FIG. 2G). Alternatively, a large insertion, deletion or translocation would also prevent amplification of alleles rs66502009 to rs4530841 contained within the same PCR product. These different possibilities may be distinguishable through analysis of developing embryos. In the case of an unrepaired break, chromosomal arms are disjoined and missegregate in mitosis. An insertion or deletion removing the primer binding site would be compatible with normal chromosome segregation patterns, while in the case of a translocation, segregation patterns would be linked to another chromosome.

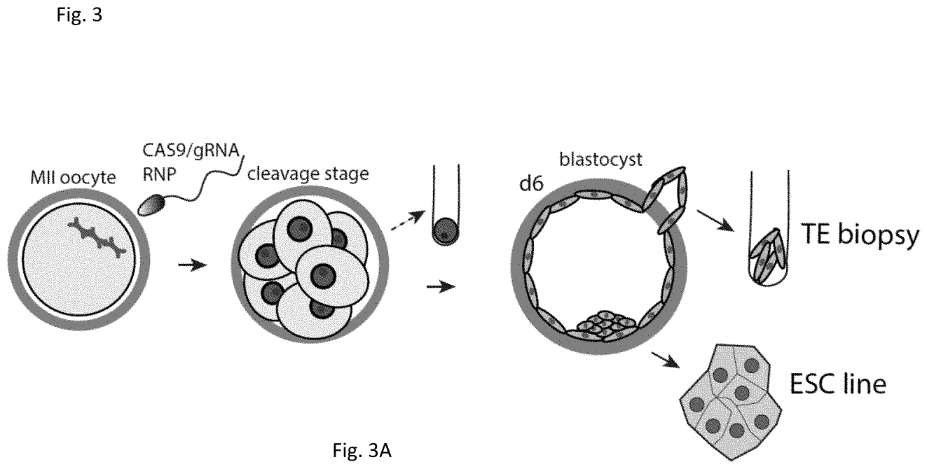

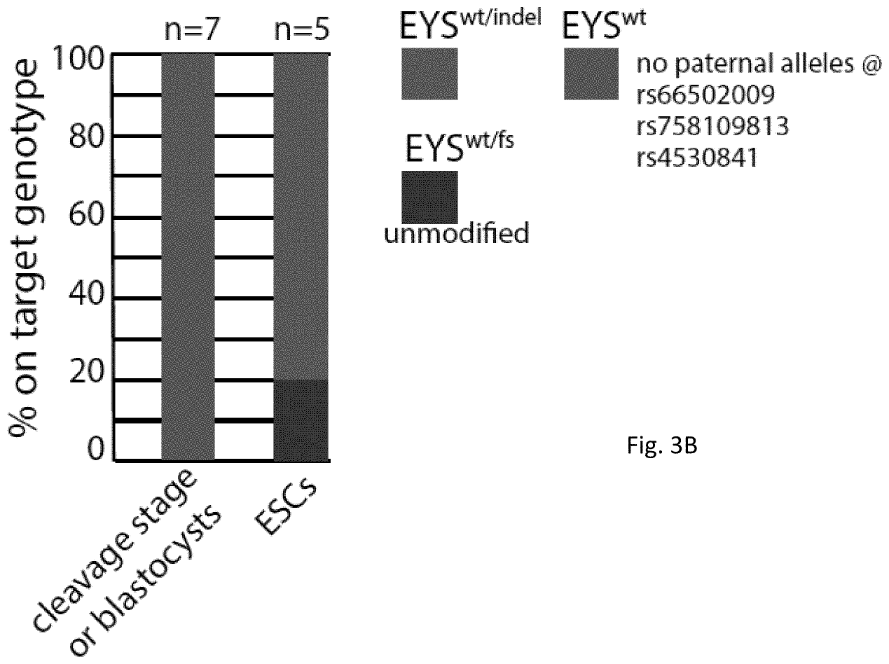

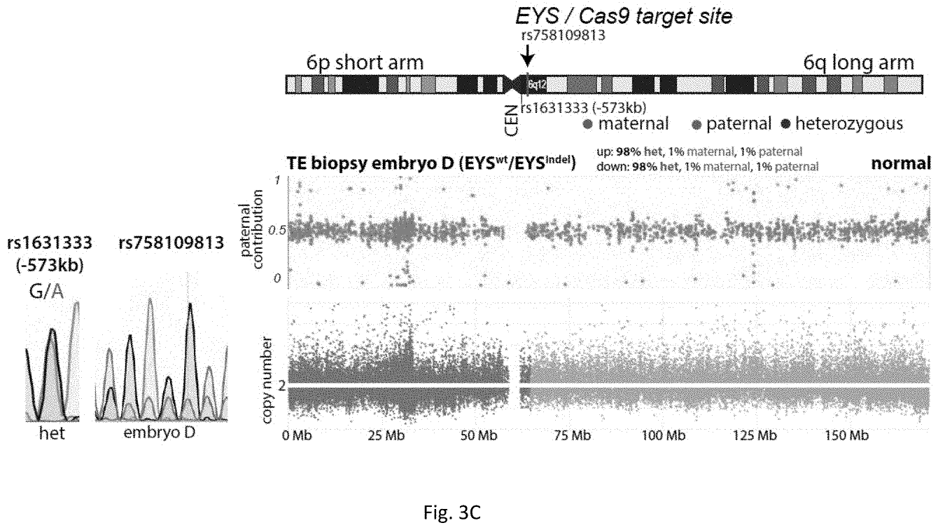

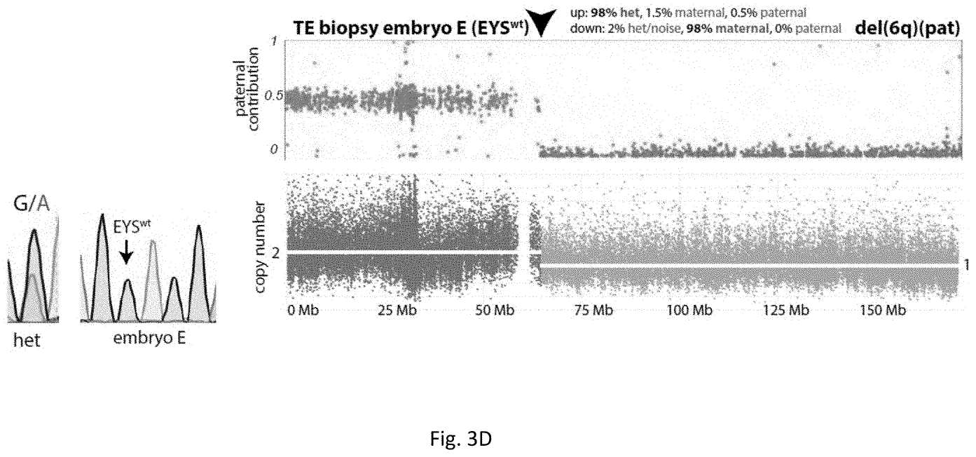

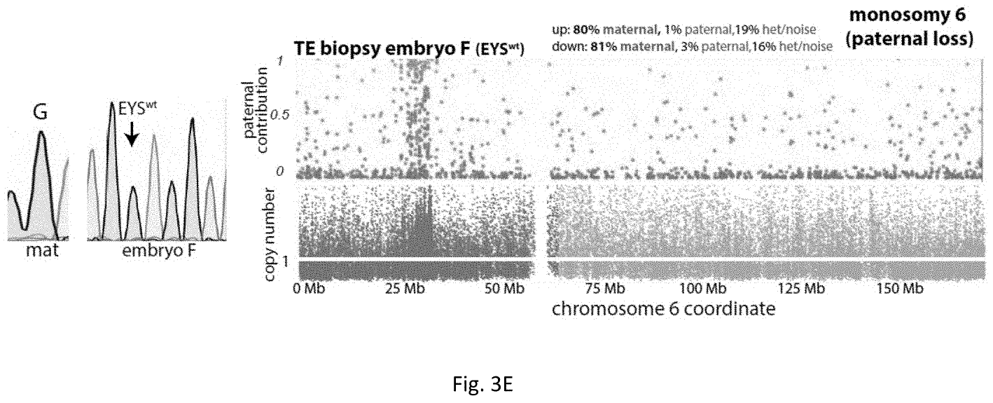

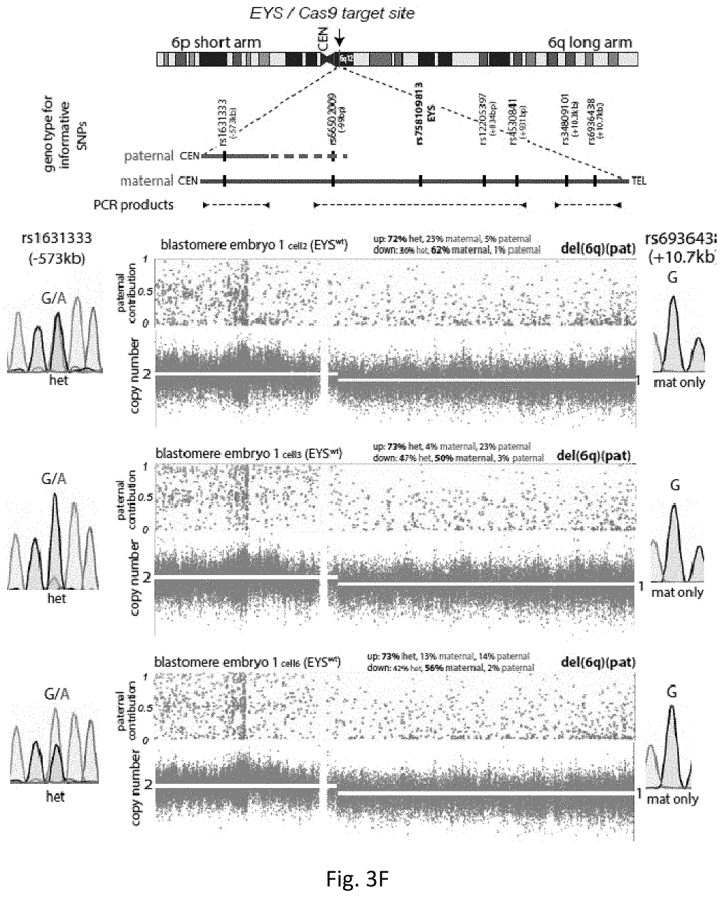

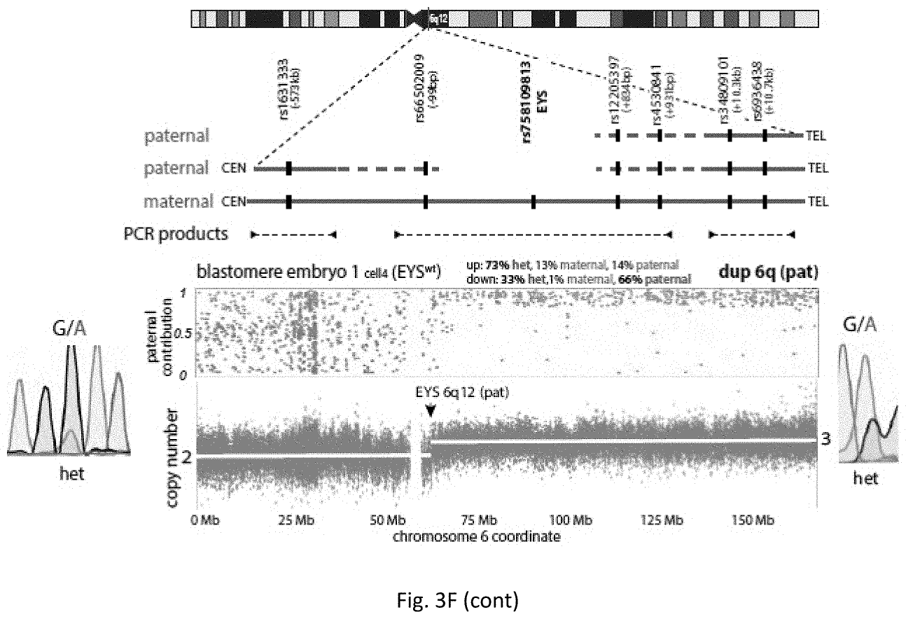

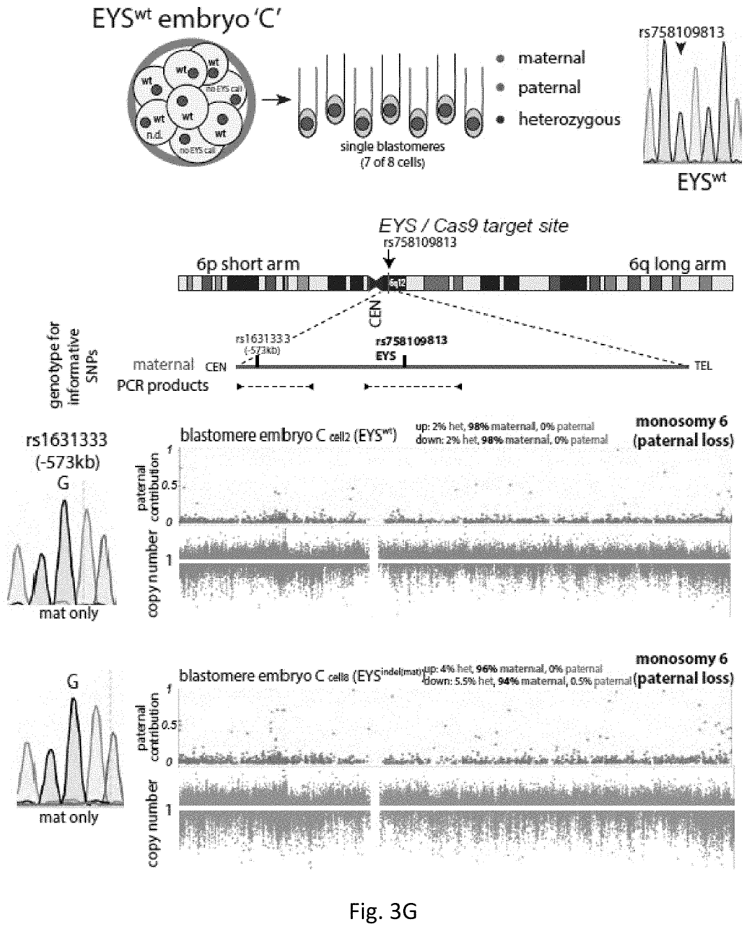

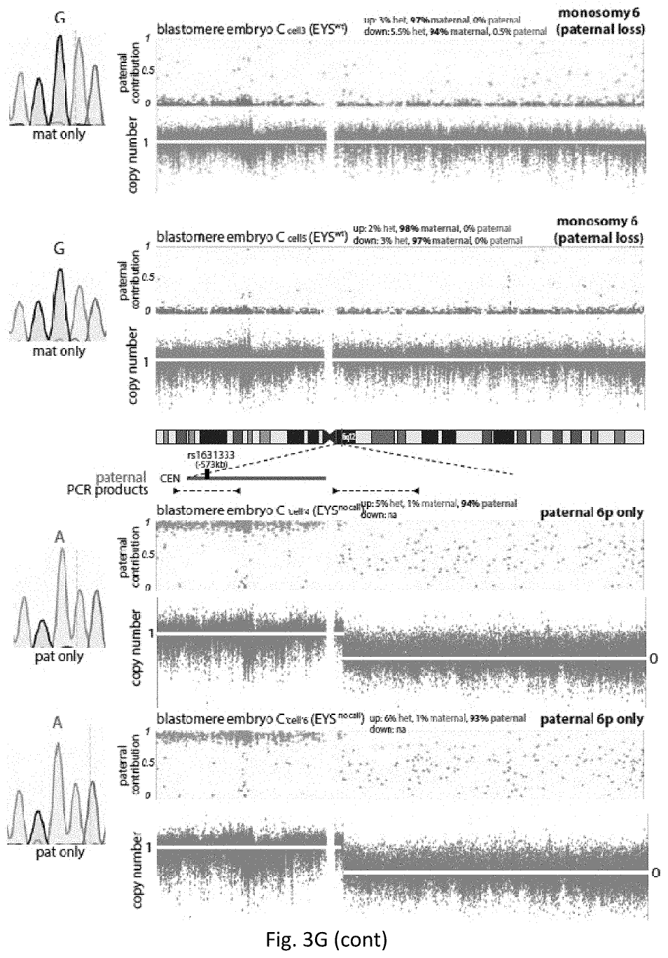

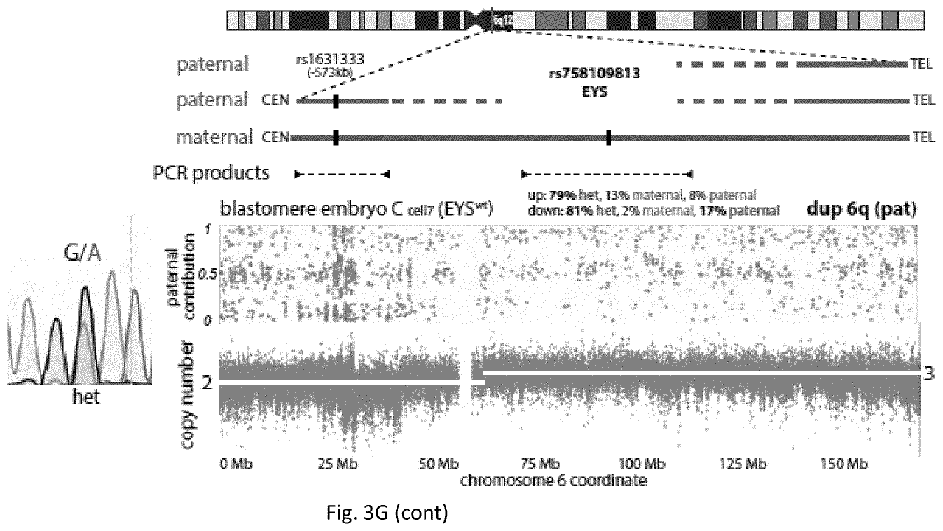

To better characterize the outcomes of a Cas9-induced DSB in zygotes and the developmental potential of edited zygotes, zygotes were allowed to develop to the blastocyst stages for biopsy, genotyping, stem cell derivation, and karyotyping (FIG. 3A). Of 18 oocytes injected with sperm and Cas9 RNP at the MII stage, 10 developed to the blastocyst stage (56%). The frequency of blastulation was within the normal range, consistent with earlier reports (Fogarty et al., 2017; Ma et al., 2017). At the cleavage stage, blastomeres from 4 embryos were biopsied, and at the blastocyst stage, trophectoderm (TE) biopsies consisting of 5-10 cells from 3 embryos were obtained (FIG. 3B). At the cleavage stage, 2 embryos only had the EYS^{wt} allele and flanking maternal SNPs (Table 2), and 2 had indels on the paternal allele. At the blastocyst stage (n=3 embryos), 2 showed only the EYS^{wt} allele (Table 2), which was confirmed by on-target NGS and allelic discrimination qPCR, and one showed an indel. Therefore, of 7 embryos analyzed with Sanger genotyping and on-target NGS, 4 (57%) were EYS^{wt} embryos (FIG. 3B).

Embryos with only maternal sequences at the mutation site could arise by interhomolog recombination giving rise to EYS^{wt/wt}, which may also lead to loss of linked paternal SNPs. Alternatively, paternal sequences could simply be lost (EYS^{wt}) due to inadequate repair of the Cas9-induced DSB. To determine whether an EYS^{wt/wt} genotype could be obtained, 10 blastocyst stage embryos, 3 of which had been genotyped by Sanger sequencing, were used for ESC derivation and 5 ESC lines were obtained. Four showed an indel at the paternal mutation site, while one maintained an unmodified paternal allele (FIG. 3B, Table 2). The derived stem cell lines were karyotypically normal and showed no evidence of off-target activity at three top off-target sites. Despite the presence of a distinct inner cell mass, no ESC lines were obtained from two EYS^{wt} blastocysts, which only had the wild-type maternal allele by TE biopsy. Instead, both plated blastocysts underwent cell death during attempted derivation, suggesting that these cells contained a lethal abnormality. Thus, there was no evidence for interhomolog repair after MII injection.

The lack of evidence for interhomolog recombination suggests other explanations are likely for the presence of maternal only sequences at the mutation site, in particular that loss of paternal sequences could simply be due to lack of repair or misrepair of the Cas9-induced DSB. The loss of the paternal allele in embryos, together with a lack of their representation in ESC lines with only the EYS^{wt} allele, led to the investigation of whether large deletions or chromosomal rearrangements accounted for the loss.

### Example 7—Segmental and Complete Loss of the Paternal Chromosome

To determine whether large deletions or chromosomal rearrangements accounted for the loss of the paternal allele, a genome-wide SNP array analysis of genomic DNA from the sperm and oocyte donors was performed. Genomic DNA from the sperm and oocyte donor was also analyzed to identify donor-specific homozygous alleles. Analysis of pluripotent stem cell lines showed that the combination of maternal and paternal genomes resulted in heterozygosity for these SNPs along the entire chromosome 6. The effect of genome amplification was controlled for and trophectoderm biopsies and single human ESCs were analyzed, since amplification of genomic DNA can alter the normal allelic ratio from 1:1 due to the stochasticity of amplification, in particular when only one cell is used. In both single amplified ESCs and trophectoderm biopsies of embryos that gave rise to the stem cell lines, heterozygous alleles were identified along the entire chromosome 6 and throughout the genome, albeit at a wider distribution. For some SNPs, only one of the alleles reaches significance of detection and thus, both paternal alleles (green dots), maternal alleles (red dots), as well as heterozygous alleles (blue dots) are called throughout chromosome 6, confirming the presence of both chromosomes. As a control, amplified genomic DNA from cumulus cells of an egg donor was also analyzed, which showed homozygous maternal alleles along chromosome 6 and throughout the genome (results not shown). These controls provide a reference for the expected appearance of amplified genomic DNA in embryo samples, and the presence or absence of paternal chromosome 6.

Next TE biopsies from blastocyst embryos that had been genotyped at EYS by Sanger sequencing were examined. Embryo D, which carried a heterozygous indel, showed heterozygosity and uniform signal intensity across chromosome 6 (FIG. 3C). In contrast, one of the two EYS^{wt} blastocysts which had failed to develop a stem cell line (embryo E) showed loss of heterozygosity spanning the region from the Cas9 cut site at EYS all the way to the telomere of the long arm of paternal chromosome 6 (FIG. 3D). The loss of heterozygosity at the EYS break site was paralleled by a loss of signal intensity due to copy number loss along the entire length of the chromosome arm 6q (FIG. 3D). Therefore, loss of genetic material, rather than break-induced replication from the paternal centromere to the telomere is responsible for the absence of paternal SNPs. Furthermore, the other EYS^{wt} blastocyst (embryo F) showed monosomy for the maternal chromosome 6, indicating loss of the entire paternal chromosome 6 (FIG. 3E). Both blastocysts were euploid for other autosomes.

To better understand the process of chromosome loss, analysis of chromosome content in blastomeres at the cleavage stage was performed. 23 individual blastomeres from 4 embryos were harvested (Table 2). Blastomeres (n=11) from two cleavage stage embryos had a maternal-only genotype at EYS, which was confirmed by on-target NGS. All four blastomeres analyzed from one embryo using SNP arrays showed segmental rearrangement of paternal chromosome 6. Of the 4 cells, 3 had losses of chromosome 6q distal of the Cas9 cleavage site, as seen by SNP array, copy number analysis and by Sanger genotyping of parent-of-origin-specific SNPs 10 kb distal to the break site (Table 2). In one cell, chromosome 6q was gained, resulting in an overrepresentation of paternal SNPs distal of EYS, and an increase in copy number. In this cell, paternal SNPs were present at rs34809101 and rs6936438, 10 kb distal of the Cas9 cleavage site (FIG. 3E, cell 4). Therefore, the loss of the paternal EYS2265fs allele can occur both through segmental losses as well as through segmental gains. These segments may not be joined with the centromere-containing p arm, and therefore missegregate in mitosis and are not detectable with primers flanking the cut site (FIG. 3E).

The other embryo had the EYS^{wt} allele at the mutation site in 5/8 blastomeres, two blastomeres had no on target genotype, and one had an indel on an EYS^{wt} allele (FIG. 3F, Table 2). Again, complementary losses and gains of paternal chromosome 6q were found. Four cells were monosomic for the maternal chromosome, one cell contained a gain of paternal chromosome 6q, and two cells contained only paternal chromosome 6p and were chaotic aneuploid. Therefore, Cas9 cleavage resulted in highly variable chromosome 6 content even in cells with the same on-target genotype EYS^{wt}. Like embryo 1 (FIG. 3E), a paternal EYS allele was not detected in blastomeres containing only chromosome segments.

Blastomeres from the remaining two cleavage stage embryos showed repair by end joining of the paternal allele. One embryo (embryo B) showed a uniform deletion of 5 bp in all (4/4) blastomeres. In the other embryo, 4/5 blastomeres (embryo A) showed a net 2 bp indel and one cell showed a wild-type only genotype by Sanger sequencing, as well as by on-target NGS (Table 2). Blastomeres from all EYS^{wt/indel} heterozygous cells showed heterozygosity and balanced copy number on chromosome 6. One EYS^{wt} blastomere had monosomy 6 due to paternal loss, and another had a segmental deletion at chromosome 6q21 on the maternal chromosome, 40 Mb telomeric of the EYS locus. Though these cleavage stage embryos had numerous mitotic aneuploidies on other autosomes, these may be spontaneous and representative of the frequent mitotic abnormalities present in cleavage stage embryos (Vanneste et al., 2009).

Of the 14 biopsies (12 blastomeres and 2 trophectoderm biopsies) with EYS^{wt} genotypes from 5 different embryos, all showed segmental or whole chromosome aneuploidies of paternal chromosome 6 (Table 2). Surprisingly, monosomy for either the long arm of chromosome 6 or the entire chromosome 6 was compatible with the development to the expanded blastocyst stage (FIG. 3D). Thus, a common outcome of Cas9 RNP injections into MII oocytes is loss of the paternal allele due to segmental and whole chromosome loss, which appear as EYS^{wt} cells in embryos characterized by on target sequencing (Table 2). These aneuploid cells were unable to produce pluripotent stem cell lines; only embryos with heterozygous indels gave rise to stem cells, even as these two genotypes each represented approximately half of preimplantation embryos (FIG. 3B). Recombination between homologous chromosomes indicated by novel combinations of maternal and paternal alleles within the same PCR product spanning rs6652009 to rs4530841 was not observed (Table 2). Taken together, the loss of the paternal EYS^{2265fs} allele after Cas9 injection at fertilization occurred through aneuploidy, not efficient interhomolog repair.

### Example 8—Cas9 RNP Injection at the 2-Cell Stage Results in Mosaicism, Chromosome Loss, and Rare Interhomolog Events

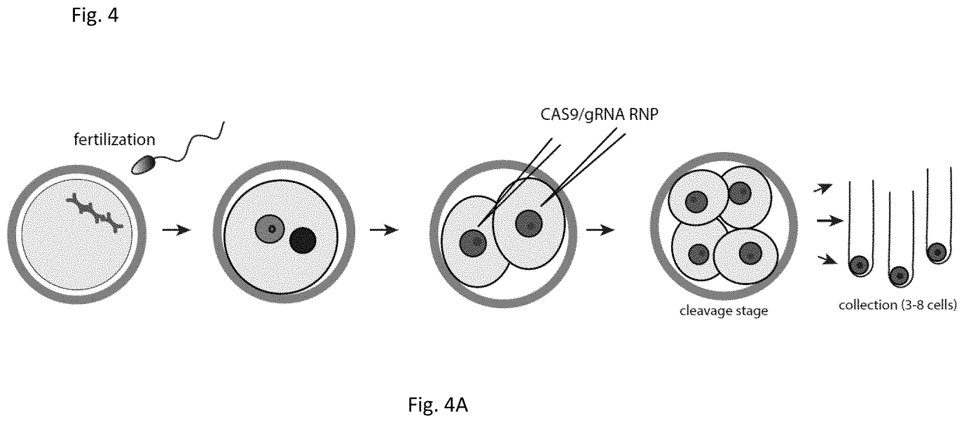

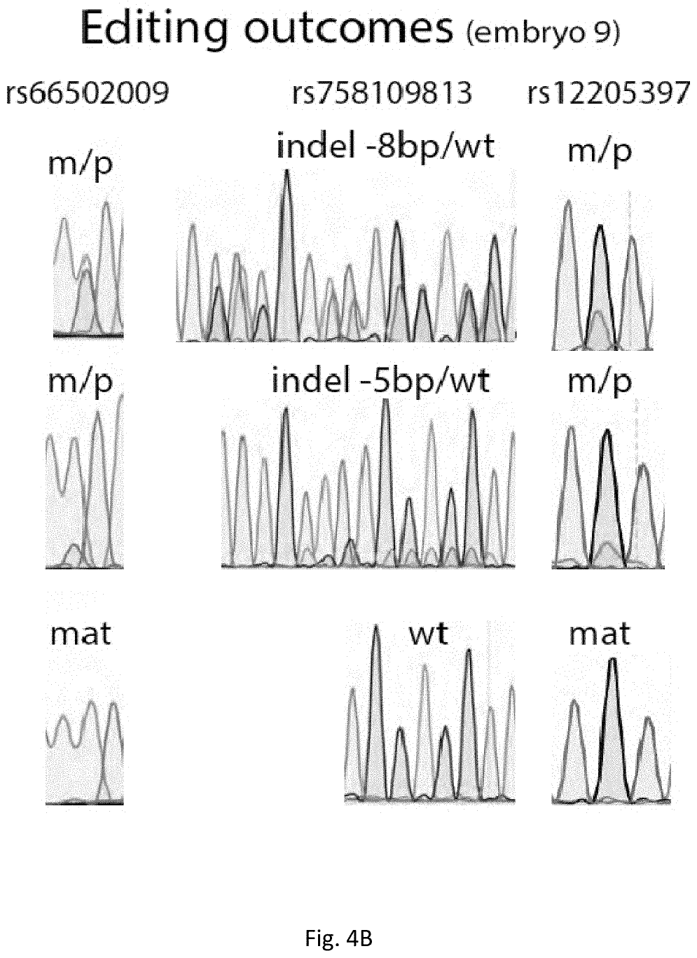

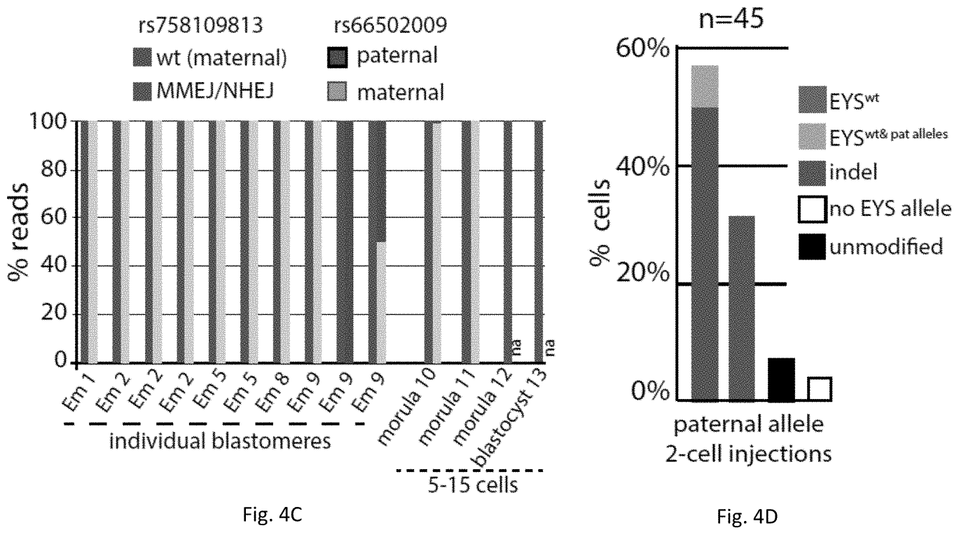

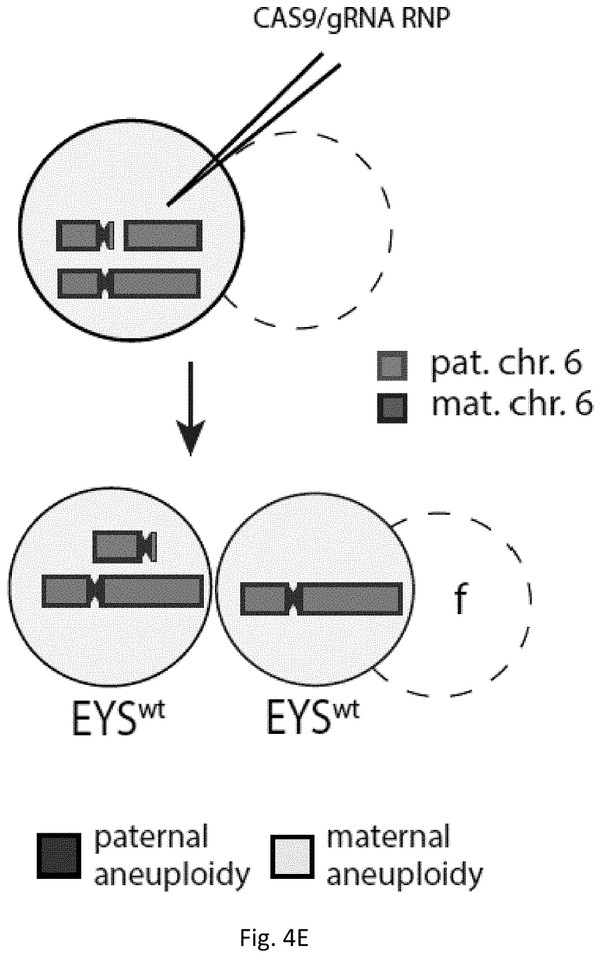

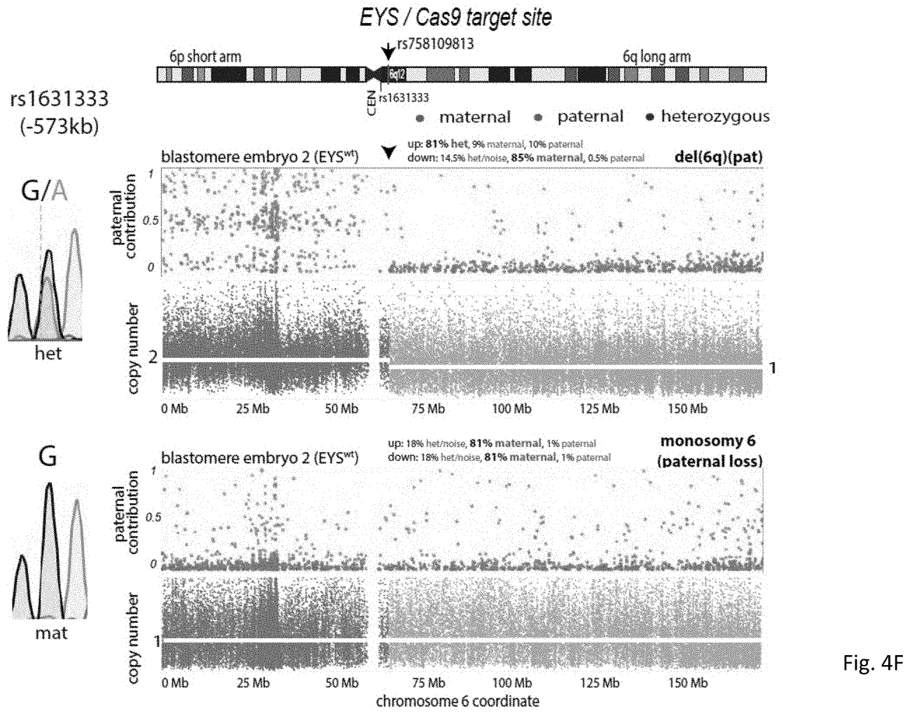

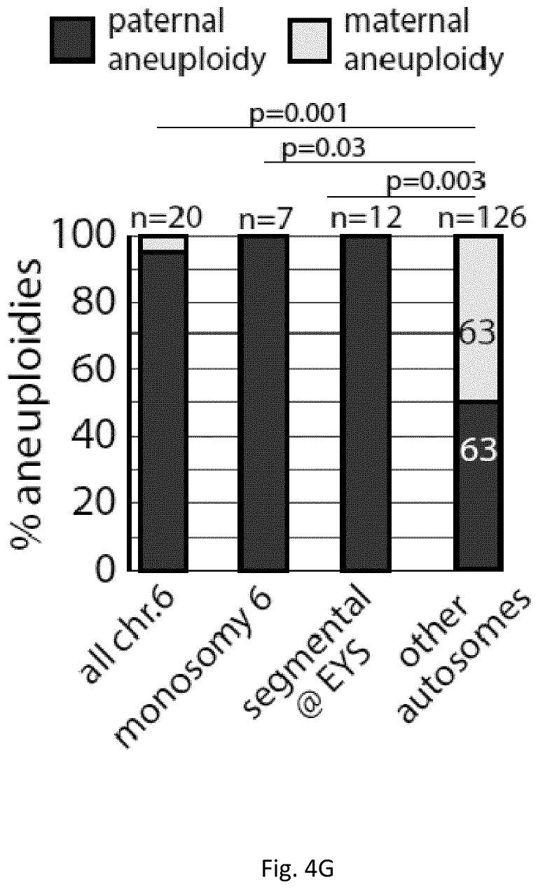

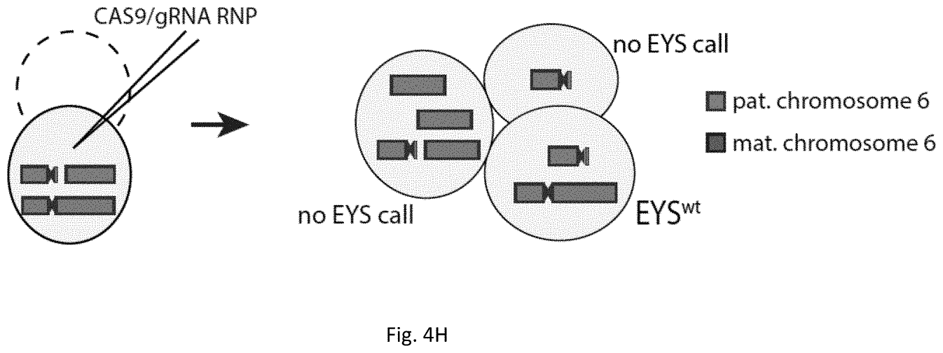

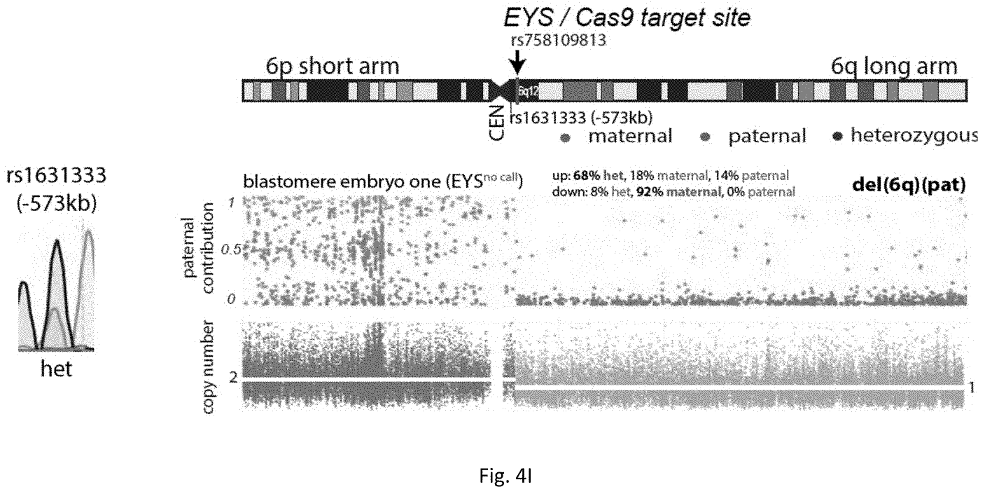

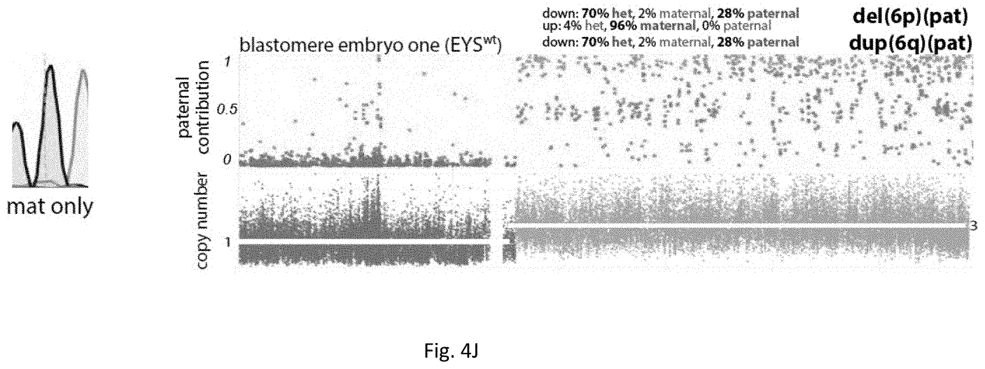

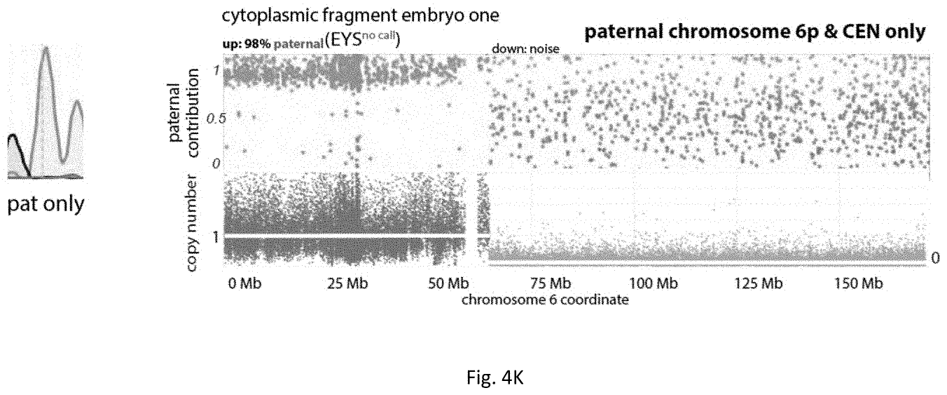

An obstacle to repairing the paternal allele through recombination between homologous chromosomes at the zygote stage is that paternal and maternal genomes are packaged in two separate nuclei (Egli et al. (2018)). To determine whether the maternal genome could provide a template for DSB repair when present with the paternal genome in the same nucleus, Cas9 RNP was injected into both cells of 13 two-cell stage embryos heterozygous for the EYS mutation and the three flanking SNPs (FIG. 4A). Single blastomeres were harvested and amplified from 11 cleavage stage embryos, and 3 embryos were analyzed at the morula and blastocyst stages using biopsies of multiple cells (FIG. 4A). Embryos dissociated to single blastomeres showed mosaicism for multiple alleles (FIG. 4B): of 45 genotyped single cells and 5 biopsies, 25 (55%) showed a maternal-only genotype at the mutation site. Of these 25 samples, 22 were maternal only at the SNPs contained with the same PCR product, rs66502009 and rs12205397, and 13 were maternal only also at all the flanking SNPs 10 kb proximal and distal of the Cas9 cleavage site, while 9 showed heterozygosity only at the 10 kb distant flanking SNPs (Table 2). On-target NGS was performed on blastomeres from 5 embryos and were confirmed as the EYS^{wt} genotype (FIG. 4C, Table 2).

In two EYS^{wt} morulas harvested as biopsies of 5-15 cells, an edited paternal allele was present with reduced representation compared to the maternal allele due to mosaicism (FIG. 4C). Interestingly, of the 25 EYS^{wt} samples, 3 cells from two different embryos showed flanking paternal SNPs on either side, which are captured within the same PCR product as EYS^{wt} (results not shown). NGS only detected a paternal allele at rs66502009, and only the maternal allele at rs758109813 (Table 2). SNPs of both maternal and paternal origin were detected throughout chromosome 6 by array analysis, demonstrating that this cell contained both paternal and maternal chromosome 6 (results not shown). The novel linkages of maternal and paternal SNPs are possible interhomolog repair events, which occurred at a frequency of ˜7% (3/45).

Furthermore, from the 45 samples, 12 (32%) showed end-joining events on the paternal allele, 3 cells showed no change, and 2 cells had no allele call even as flanking SNPs were detected (FIG. 4D, Table 2).

In the remaining 3 cells, heterozygous indels on an allele with a wild-type SNP rs758109813 were observed. One such edit had also been observed in the MII injections in ‘embryo C’. Therefore, Cas9 RNP cut the maternal allele with an efficiency of 4/84 (˜5%), while in the same samples, editing of the paternal allele was 78/84 (93%). Thus, the presence of the suboptimal PAM site GAG allowed some cleavage of the maternal allele in the embryo, though with much lower efficiency than on the paternal allele.

To determine whether EYS^{wt} blastomeres were caused by paternal chromosome loss, blastomeres of two embryos were tested after a single mitosis post-Cas9/RNP injection for heterozygosity using SNP arrays (FIGS. 4E-4K). In one embryo, two sister blastomeres with an EYS^{wt} genotype showed loss of chromosome 6q and its sister cell showed monosomy 6 due to loss of the paternal chromosome (FIG. 4F, FIG. 4G). For a remaining third cell of the same embryo that had not divided upon injection and with an EYSwt genotype, SNP array analysis was not successful.

In the other embryo, complementary loss of either paternal chromosomal arm 6q or 6p plus the centromere with breakpoints at the EYS locus was found (FIGS. 4H-K). The long arm that was lost in one blastomere was gained in the other, as seen by an increased representation of paternal SNPs in one of the two sister blastomeres on chromosome 6q (FIG. 4J) and reciprocal copy number gain and loss on the q or the p arm (results not shown). In addition, one ‘cell’ contained only chromosome 6p, no signal from chromosome 6q, and no other genomic DNA (FIG. 4K). The exclusion of chromosomal arms in cytoplasmic fragments may be one mechanism of their elimination from the embryo. The reciprocal losses and gains of chromosomal arms were also observed by Sanger sequencing of rs1631333, located 573 kb from the Cas9 cleavage site towards the centromere (FIG. 4I-K, Table 2). The product(s) of another cell of the injected 2-cell embryo could not be determined. Loss of heterozygosity across the centromere is inconsistent with copy-neutral mitotic recombination, providing further support that the loss of paternal alleles occurred through the loss of genetic material rather than interhomolog recombination. Thus, Cas9 RNP injection at the 2-cell stage can result in the loss of the paternal chromosome through missegregation in mitosis due to an unrepaired DSB at the EYS locus.

Considering karyotypes of all embryos from injections at fertilization or at the 2-cell stage, all cells and samples (19/19) with loss of EYS^{2265fs} and of the flanking paternal alleles showed paternal specific abnormalities on chromosome 6. The one exception was a blastomere after 2-cell stage injection that contained flanking paternal alleles and may be due to interhomolog repair. Prior to the first cells division, the loss of paternal genotype may also be due to an unrepaired or misrepaired break in a nucleus with a normal karyotype (FIG. 2G), resulting in aneuploidies only after mitosis. Aneuploidies of chromosome 6 were significantly enriched on the paternal chromosome for both segmental errors as well as whole chromosome loss (FIG. 4G). In contrast aneuploidies acquired after fertilization on the other autosomes equally affected both paternal and maternal chromosomes (50% vs. 50%) (FIG. 4G). Thus, Cas9-induced cleavage in human embryos results in allele-specific segmental and whole chromosome errors beyond what is observed in normal development.

### Example 9—Cas9 Off-Target Effects Include Indels and Chromosome Loss

Spontaneous aneuploidies are common in human cleavage stage embryos (Vanneste et al., 2009). In the dataset of this study, 4 of 11 embryos injected at the MII stage with Cas9 RNP contained aneuploidies on autosomes other than chromosome 6, which could be spontaneous or Cas9 induced if off-target sites are present. Thus, segmental errors were focused on to evaluate off-target aneuploidies, as the genomic coordinates of chromosomal break points can be correlated with the location of predicted off-target sites.

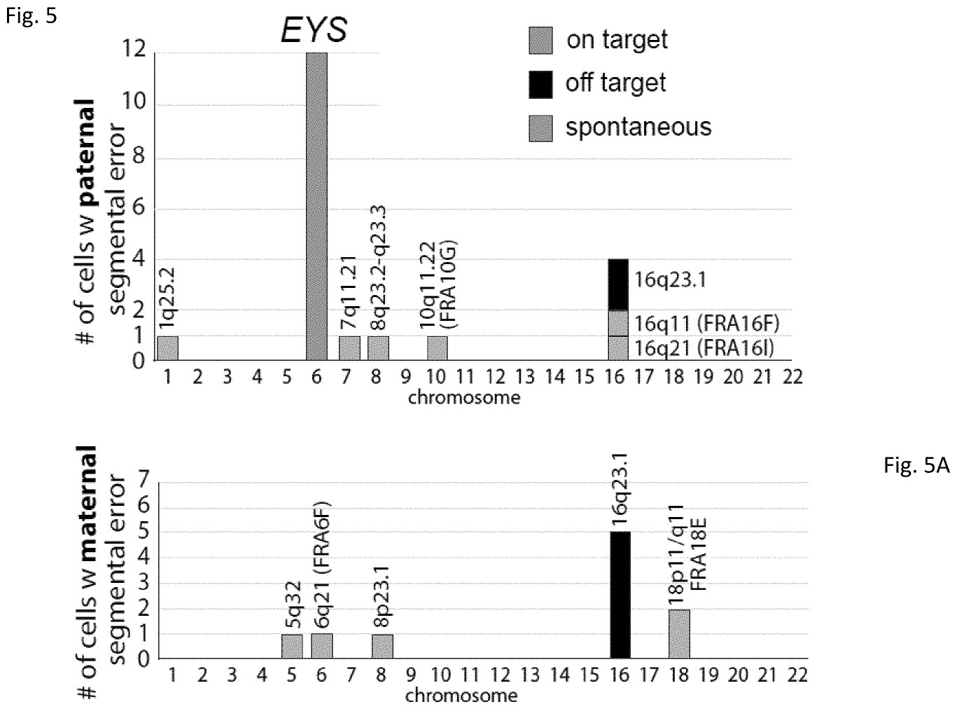

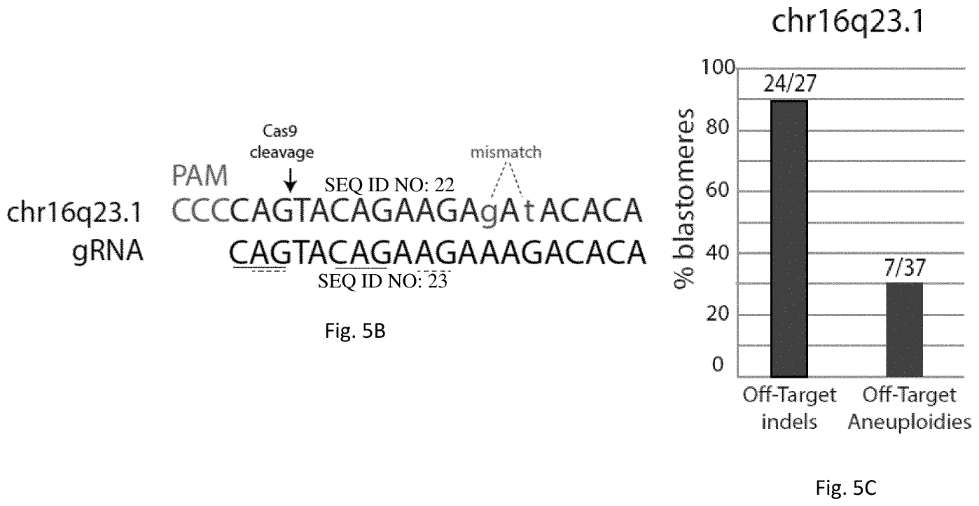

Segmental errors of either paternal or maternal origin were found at 11 different sites (FIG. 5A). All but two of the segmental errors were found only once, suggesting they occurred in a single cell during the second or third cell cycle after fertilization and Cas9 injection. Five mapped to common fragile sites. For instance, one site on the maternal chromosome 6q mapped to FRA6F telomeric of EYS and was found in only one of 5 cells of an embryo injected with Cas9 RNP at fertilization.

However, one site of segmental loss on chromosome 16 was recurrent in 3 of 7 cleavage stage embryos examined (FIG. 5A, Table 2). To determine concordance with Cas9 off-target sites, 9 predicted off target candidate sites for indels were evaluated. One gRNA had 2 mismatches (FIG. 5B), while all others had 3 or more. PCR and Sanger sequencing in 4 stem cell lines and 3 blastocysts revealed that only one site on chromosome 16923.1 at location chr16:74344276 (hg37), the site with 2 mismatches to the gRNA showed indels in 4 of 7 samples. The site is concordant with the cytological location of recurrent segmental errors on the same chromosome: chromosomal break sites in 7 different cells mapped between chr16:74 Mb-74.5 Mb at 16922.3-23.1. None of the other predicted off-target sites were concordant with the location of singular segmental errors, which were hence considered spontaneous (FIG. 5A). The off-target site on chromosome 16 was then further examined for indels in a total of 27 cleavage stage blastomeres of 7 embryos. Off-target indels were identified in 24 blastomeres, with one cell carrying two different indels (FIG. 5C, Table 2). In cleavage stage embryos derived after MII injections of Cas9 RNP, 3/4 embryos showed mosaic indels and just one was uniform (FIG. 5D, Table 2). To determine the timing of off-target activity, 16 isolated haploid nuclei containing either a maternal or a paternal genome were analyzed at 20 h post fertilization and Cas9 RNP injection (FIG. 2H, FIG. 5E). Of the 16 nuclei, 5 showed heterozygous indels, 9 were unmodified, and 2 were undetectable (FIG. 5E). As was the case for the on-target location, 5 bp and 8 bp deletions through MMEJ were again observed (FIG. 5F, Table 2). Interestingly (5/5) modified nuclei were heterozygous at the 1-cell stage, carrying either an indel/wt allele (2 nuclei), or two different indels (3 nuclei), resulting from independent end joining events on sister chromatids (FIG. 5E). Thus, Cas9 cleavage at the off-target location occurred predominantly after the first S-phase, resulting in mosaicism.

Segmental chromosomal losses at chromosome 16923.1 were found in 7/37 cells (19%) in 3 of 7 examined cleavage stage embryos, all of which were mosaic. One embryo, embryo one, derived after Cas9 RNP injection at the 2-cell stage, carried different modifications on all 4 sister chromatids in both daughter cells (FIG. 5F). One cell showed maternal segmental loss and an indel on the other, paternal allele, while the other carried paternal segmental loss and a different indel on the maternal allele. The parental origin of the chromosomes with the segmental error is identified through homozygous parent-of-origin specific SNPs. Cells with an indel and a segmental loss on the other chromosome appeared homozygous in a Sanger sequencing assay, but are in fact hemizygous (FIG. 5F). Mosaic chromosome content and loss of heterozygosity of chromosome 16 was also seen in embryo C, obtained after MII injection of Cas9 RNP (results not shown). All blastomeres carried a 6 bp insertion on one chromosome, which appeared heterozygous in some cells, and homozygous in others, depending on chromosomal content. Taken together, off-target activity of Cas9 on chromosome 16 results in both indels, as well as in segmental chromosomal changes in the human embryo.

TABLE 2
Results from On Target Sanger Sequencing, On Target Deep Sequencing,
Allelic Discrimination qPCR, and Karyotyping using SNP Arrays

| | shown | | | | | | | | | | |
|---|---|---|---|---|---|---|---|---|---|---|---|
| | in | | | | | | | | | | |
| | Figure | | | | | | | | | | |
| | (all | | | | | | | | | | |
| | samples | | | | | | | | | | |
| | relate to | | | | | | | | | rs66502009 | |
| | FIG. 1I, | maternal | | | | | | | | (−99p) | |
| | FIG. 1J, | egg | | | | | rs1482454 | rs9362339 | | deep | |
| Cas9 RNP | FIG. 4G, | genome | | Chr6 (full | rs1631333 | rs1482455 | (− | (− | rs66502009 | sequencing | |
| injection at | FIG. 5A) | origin | collection as | kayotypes) | (−573 kb) | (−11.2 kb) | 10.88 kb) | 10.71 kb) | (−99 bp) | (FIG. 4C,) | |
| Androgenesis | FIG. 2A, | | | | | | | | | | |
| @ 20 h (no | FIG. 2D | | | | | | | | | | |
| maternal | | | | | | | | | | | |
| genome) | | | | | | | | | | | |
| na | | na | gDNA sperm | normal | na | T/T | G/G | T/T | C/C | nd | |
| | | | donor | | | | | | | | |
| MII | | na | 20 h zygote | nd | nd | nd | nd | nd | C | nd | |
| androgenesis | | | | | | | | | | | |
| MII | | na | 20 h zygote | nd | nd | nd | nd | nd | f | nd | |
| androgenesis | | | | | | | | | | | |
| MII | | na | 20 h zygote | nd | nd | nd | nd | nd | C | nd | |
| androgenesis | | | | | | | | | | | |
| MII | | na | 20 h zygote | nd | nd | nd | nd | nd | C | nd | |
| androgenesis | | | | | | | | | | | |
| MII | | na | 20 h zygote | nd | nd | nd | nd | nd | f | nd | |
| androgenesis | | | | | | | | | | | |
| MII | | na | 20 h zygote | nd | nd | nd | nd | nd | C | nd | |
| androgenesis | | | | | | | | | | | |
| MII | | na | 20 h zygote | nd | nd | nd | nd | nd | C | nd | |
| androgenesis | | | | | | | | | | | |
| MII | | na | 20 h zygote | nd | nd | nd | nd | nd | C | nd | |
| androgenesis | | | | | | | | | | | |
| control | FIG. 1A | | | | | | | | | | |
| genotyping | | | | | | | | | | | |
| na | | na | gDNA sperm | normal | A/A | T/T | G/G | T/T | C/C | na | |
| | | | donor | | | | | | | | |
| na | | donor G | gDNA egg | nd | nd | C/C | A/A | C/C | A/A | na | |
| | | | donor G | | | | | | | | |
| control - none | | donor G | 2-cell d2 | nd | G/A | C/T | A/G | C/T | A/C | nd | |
| | | | control embryo | | | | | | | | |
| | | | 1 | | | | | | | | |
| control - none | | donor G | 2-cell d2 | nd | nd | C/T | A/G | C/T | A/C | nd | |
| | | | control embryo | | | | | | | | |
| | | | 1 | | | | | | | | |
| na | | donor A | gDNA egg | normal | na | C/T | A/G | C/T | A/A | na | |
| | | | donor A | | | | | | | | |
| control - none | | donor A | 2-cell d2 | nd | nd | nd | nd | nd | A/C | nd | |
| | | | control embryo | | | | | | | | |
| | | | 2 | | | | | | | | |
| na | | donor C | gDNA egg | normal | nd | nd | nd | nd | A/A | na | |
| | | | donor C | | | | | | | | |
| control - none | | donor C | single ESC | normal | nd | nd | nd | nd | A/C | nd | |
| | | | eysES6 | | | | | | | | |
| control - none | | donor C | single ESC | normal | nd | nd | nd | nd | A/C | nd | |
| | | | eysES6 | | | | | | | | |
| control - none | | donor C | single ESC | nd | nd | nd | nd | nd | A/C | nd | |
| | | | eysES6 | | | | | | | | |
| control - none | | donor C | single ESC | nd | nd | nd | nd | nd | A/C | nd | |
| | | | eysES6 | | | | | | | | |
| 2PN analysis @ | FIG. 2C, | | | | | | | | | | |
| 20 h after MII | FIG. 2E | | | | | | | | | | |
| injection | | | | | | | | | | | |
| na | | na | gDNA sperm | normal | A/A | T/T | G/G | T/T | C/C | na | |
| | | | donor | | | | | | | | |
| na | | donor A | gDNA egg | normal | G/G | C/T | A/G | C/T | A/A | na | |
| | | | donor A | | | | | | | | |
| MII | | donor A | zygote 1 | normal | G/A | C | A | C | A | A (100%) | |
| MII | | donor A | zygote 2 | f | nd | T | G | T | A | A (100%) | |
| na | | donor B | gDNA egg | normal | nd | C/T | A/G | C/T | A/C | na | |
| | | | donor B | | | | | | | | |
| MII | | donor B | zygote 3 | nd | nd | C/T | A/G | C/T | nd | nd | |
| 2PN individual | FIG. 2B, | | | | | | | | | | |
| pronuclei @ | FIG 2F, | | | | | | | | | | |
| 20 h | FIG. 5E | | | | | | | | | | |
| na | | na | gDNA sperm | normal | A/A | T/T | G/G | T/T | C/C | na | |
| | | | donor | | | | | | | | |
| na | | na | gDNA egg | normal | G/G | C/T | A/G | C/T | A/C | na | |
| | | | donor B | | | | | | | | |
| mMII - 1 | | donor B | 20 h zygote, | normal | G | T | G | T | C | nd | |
| | | | individual | | | | | | | | |
| | | | nuclei | | | | | | | | |
| pMII - 1 | | na | 20 h zygote, | normal | A | T | G | T | C | nd | |
| | | | individual | | | | | | | | |
| | | | nuclei | | | | | | | | |
| mMII - 2 | | donor B | 20 h zygote, | nd | G | T | G | T | C | nd | |
| | | | individual | | | | | | | | |
| | | | nuclei | | | | | | | | |
| pMII - 2 | | na | 20 h zygote, | normal | A | T | G | T | C | nd | |
| | | | individual | | | | | | | | |
| | | | nuclei | | | | | | | | |
| mMII - 3 | | donor B | 20 h zygote, | normal | G | T | G | T | C | nd | |
| | | | individual | | | | | | | | |
| | | | nuclei | | | | | | | | |
| pMII - 3 | | na | 20 h zygote, | normal | A | T | G | T | C | nd | |
| | | | individual | | | | | | | | |
| | | | nuclei | | | | | | | | |
| mMII - 4 | | donor B | 20 h zygote, | normal | G | T | G | T | C | nd | |
| | | | individual | | | | | | | | |
| | | | nuclei | | | | | | | | |
| pMII - 4 | | na | 20 h zygote, | normal | A | T | G | T | C | nd | |
| | | | individual | | | | | | | | |
| | | | nuclei | | | | | | | | |
| mMII - 5 | | donor B | 20 h zygote, | normal | G | T | G | T | C | nd | |
| | | | individual | | | | | | | | |
| | | | nuclei | | | | | | | | |
| pMII - 5 | | na | 20 h zygote, | normal | A | T | G | T | f | nd | |
| | | | individual | | | | | | | | |
| | | | nuclei | | | | | | | | |
| mMII - 6 | | donor B | 20 h zygote, | normal | G | T | G | T | C | nd | |
| | | | individual | | | | | | | | |
| | | | nuclei | | | | | | | | |
| pMII - 6 | | na | 20 h zygote, | normal | A | T | G | T | C | nd | |
| | | | individual | | | | | | | | |
| | | | nuclei | | | | | | | | |
| mMII - 7 | | donor B | 20 h zygote, | normal | G | nd | nd | nd | C | nd | |
| | | | individual | | | | | | | | |
| | | | nuclei | | | | | | | | |
| pMII - 7 | | na | 20 h zygote, | normal | A | T | G | T | f | nd | |
| | | | individual | | | | | | | | |
| | | | nuclei | | | | | | | | |
| mMII - 8 | | donor B | 20 h zygote, | nd | G | nd | nd | nd | C | nd | |
| | | | individual | | | | | | | | |
| | | | nuclei | | | | | | | | |
| pMII - 8 | | na | 20 h zygote, | normal | A | T | G | T | C | nd | |
| | | | individual | | | | | | | | |
| | | | nuclei | | | | | | | | |
| Blastomere | FIG. 3B, | | | | | | | | | | |
| collection | FIG. 5C- | | | | | | | | | | |
| after | D | | | | | | | | | | |
| MII injection | | | | | | | | | | | |
| na | | na | gDNA sperm | normal | A/A | T/T | G/G | T/T | C/C | na | |
| | | | donor | | | | | | | | |
| na | | donor A | gDNA egg | normal | G/G | C/T | A/G | C/T | A/A | na | |
| | | | donor A | | | | | | | | |
| | FIG. 3E | | embryo 1 (6 of | | | | | | | | |
| | | | 7 cells) | | | | | | | | |
| MII | | donor A | MII blastomere | f | f | f | f | f | A | A (100%) | |
| | | | embryo 1 | | | | | | | | |
| MII | | donor A | MII blastomere | del 6q | G/A | T | G | T | A | A (100%) | |
| | | | embryo 1 | (pat) | | | | | | | |
| MII | | donor A | MII blastomere | del 6q | G/A | T | G | T | A | A (91%) / | |
| | | | embryo 1 | (pat) | | | | | | G (9%) | |
| MII | | donor A | MII blastomere | dup 6q | G/A | T | G | T | A | A (100%) | |
| | | | embryo 1 | (pat) | | | | | | | |
| MII | | donor A | MII blastomere | del 6q | G/A | T | G | T | A | A (100%) | |
| | | | embryo 1 | (pat) | | | | | | | |
| MII | | donor A | MII blastomere | nd | G/A | T | G | T | A | nd | |
| | | | embryo 1 | | | | | | | | |
| na | | donor D | gDNA egg | normal | G/G | T/T | G/G | T/T | C/C | na | |
| | | | donor D | | | | | | | | |
| | | | embryo A (5 | | | | | | | | |
| | | | cells) | | | | | | | | |
| MII | | donor D | MII blastomere | normal | G/A | T | G | T | C/C | C (100%) | |
| | | | embryo A (cell | EYS locus, | | | | | | | |
| | | | 1) | del6q21- | | | | | | | |
| | | | | 27 (mat) | | | | | | | |
| MII | | donor D | MII blastomere | normal | G/A | T | G | T | C/C | C (100%) | |
| | | | embryo A (cell | | | | | | | | |
| | | | 2) | | | | | | | | |
| MII | | donor D | MII blastomere | normal | G/A | T | G | T | C/C | C (100%) | |
| | | | embryo A (cell | | | | | | | | |
| | | | 3) | | | | | | | | |
| MII | | donor D | MII blastomere | normal | G/A | T | G | T | C/C | C (100%) | |
| | | | embryo A (cell | | | | | | | | |
| | | | 4) | | | | | | | | |
| MII | | donor D | MII blastomere | monosomy | G | T | G | T | C | C (100%) | |
| | | | embryo A (cell | 6 (pat) | | | | | | | |
| | | | 5, A8) | | | | | | | | |
| | | | Embryo B (4 | | | | | | | | |
| | | | cells) | | | | | | | | |
| MII | | donor D | MII blastomere | normal | G/A | T | G | T | C/C | C (100%) | |
| | | | embryo B | | | | | | | | |
| | | | (cell1) | | | | | | | | |
| MII | | donor D | MII blastomere | normal | G/A | T | G | T | C/C | C (100%) | |
| | | | embryo B | | | | | | | | |
| | | | (cell2) | | | | | | | | |
| MII | | donor D | MII blastomere | normal | G/A | T | G | T | C/C | C (100%) | |
| | | | embryo B | | | | | | | | |
| | | | (cell3) | | | | | | | | |
| MII | | donor D | MII blastomere | normal | G/A | T | G | T | C/C | C (100%) | |
| | | | embryo B | | | | | | | | |
| | | | (cell4) | | | | | | | | |
| | | | Embryo C (8 | | | | | | | | |
| | | | cells) | | | | | | | | |
| MII | | donor D | MII blastomere | nd | nd | nd | nd | nd | C | nd | |
| | | | embryo C | | | | | | | | |
| | | | (cell1) | | | | | | | | |
| MII | | donor D | MII blastomere | monosomy | G | nd | nd | nd | C | C (100%) | |
| | | | embryo C (cell | 6 (pat) | | | | | | | |
| | | | 2) | | | | | | | | |
| MII | | donor D | MII blastomere | monosomy | G | nd | nd | nd | C | C (100%) | |
| | | | embryo C (cell | 6 (pat) | | | | | | | |
| | | | 3) | | | | | | | | |
| MII | | donor D | MII blastomere | paternal | A | f | f | f | f | na | |
| | | | embryo C (cell | 6p only | | | | | | | |
| | | | 4) | | | | | | | | |
| MII | | donor D | MII blastomere | monosomy | G | nd | nd | nd | C | C (100%) | |
| | | | embryo C (cell | 6 (pat) | | | | | | | |
| | | | 5) | | | | | | | | |
| MII | | donor D | MII blastomere | paternal | A | f | f | f | f | na | |
| | | | embryo C (cell | 6p only | | | | | | | |
| | | | 6) | | | | | | | | |
| MII | | donor D | MII blastomere | dup 6q | G/A | T | G | T | C | C (100%) | |
| | | | embryo C (cell | (pat) | | | | | | | |
| | | | 7) | | | | | | | | |
| MII | | donor D | MII blastomere | monosomy | G | nd | nd | nd | C | nd | |
| | | | embryo C (cell | 6 (pat) | | | | | | | |
| | | | 8) | | | | | | | | |
| Trophectoderm | FIG. 3, | | | | | | | | | | |
| Biopsy after | | | | | | | | | | | |
| MII injection | | | | | | | | | | | |
| na | | na | gDNA sperm | normal | A/A | T/T | G/G | T/T | C/C | na | |
| | | | donor | (46, XY) | | | | | | | |
| na | | donor D | gDNA egg | normal | G/G | T/T | G/G | T/T | C/C | na | |
| | | | donor D | (46, XX) | | | | | | | |
| MII | FIG. 3C | donor D | TE biopsy d6 | normal | G/A | T | G | T | C/C | nd | |
| | | | (embryo D) x2 | | | | | | | | |
| MII | FIG. 3D | donor D | TE biopsy d6 | del 6q | G/A | T | G | T | C | C (100%) | |
| | | | (embryo E) | (pat) | | | | | | | |
| MII | FIG. 3E | donor D | TE biopsy d6 | monosomy | G | T | G | T | C | C (100%) | |
| | | | (embryo F) x2 | 6 (pat) | | | | | | | |
| TE biopsies | | | | | | | | | | | |
| without Sanger | | | | | | | | | | | |
| genotype | | | | | | | | | | | |
| MII | | donor C | TE biopsy | normal | nd | nd | nd | nd | nd | nd | |
| | | | (embryo 4) | | | | | | | | |
| | | | matches | | | | | | | | |
| | | | eysES4 x2 | | | | | | | | |
| MII | | donor C | TE biopsy | normal | nd | nd | nd | nd | nd | nd | |
| | | | (embryo 10) x2 | | | | | | | | |
| MII | | donor C | TE biopsy | normal | nd | nd | nd | nd | nd | nd | |
| | | | (embryo 8) | | | | | | | | |
| | | | matches | | | | | | | | |
| | | | eysES8 | | | | | | | | |
| MII | | donor C | TE biopsy | normal | nd | nd | nd | nd | nd | nd | |
| | | | (embryo 5) | | | | | | | | |
| | | | matches | | | | | | | | |
| | | | eysES5 | | | | | | | | |
| | | | x2 = two | | | | | | | | |
| | | | biopsies | | | | | | | | |
| | | | collected with | | | | | | | | |
| | | | consistent | | | | | | | | |
| | | | results | | | | | | | | |
| ESC derivation | FIG. 3B, | | | | | | | | | | |
| after MI1 | | | | | | | | | | | |
| injection | | | | | | | | | | | |
| na | | na | gDNA sperm | normal | A/A | T/T | G/G | T/T | C/C | na | |
| | | | donor | | | | | | | | |
| na | | donor C | gDNA egg | normal | nd | nd | nd | nd | A/A | na | |
| | | | donor C | | | | | | | | |
| MII | | donor C | eysESC4 (from | normal | G/A | C/T | A/G | C/T | A/C | nd | |
| | | | embryo 4) | | | | | | | | |
| MII | | donor C | eysESC5 (from | normal | nd | C/T | A/G | C/T | A/C | nd | |
| | | | embryo 5) | | | | | | | | |
| MII | | donor C | eysESC6 | normal | G/A | C/T | A/G | C/T | A/C | A/C | |
| MII | | donor C | eysESC8 (from | normal | nd | C/T | A/G | C/T | A/C | nd | |
| | | | embryo 8) | | | | | | | | |
| na | | donor D | gDNA egg | normal | G/G | T/T | G/G | T/T | C/C | na | |
| | | | donor D | | | | | | | | |
| MII | | donor D | eysESC9 | nd | G/A | nd | nd | nd | C/C | nd | |
| | | | (From embryo | | | | | | | | |
| | | | D) | | | | | | | | |
| na | | donor I | gDNA egg | nd | nd | C/C | A/A | C/C | A/A | na | |
| | | | donor I | | | | | | | | |
| control - none | | donor I | eysESC 1 | nd | nd | C/T | A/G | C/T | A/C | nd | |
| 2-cell stage | FIG. 4, | | | | | | | | | | |
| injections | FIG. 5C | | | | | | | | | | |
| na | | na | gDNA sperm | normal | A/A | T/T | G/G | T/T | C/C | na | |
| | | | donor | | | | | | | | |
| na | | donor G | gDNA egg | nd | nd | C/C | A/A | C/C | A/A | na | |
| | | | donor G | | | | | | | | |
| | | | embryo 4 (3 of | | | | | | | | |
| | | | 4 cells) | | | | | | | | |
| 2-cell | | donor G | embryo 4 | nd | nd | nd | nd | nd | A | nd | |
| 2-cell | | donor G | embryo 4 | nd | nd | C | A | C | A/A | nd | |
| 2-cell | | donor G | embryo 4 | nd | nd | C/T | A/G | C/T | A/C | nd | |
| na | | donor A | gDNA egg | normal | G/G | C/T | A/G | C/T | A/A | nd | |
| | | | donor A | | | | | | | | |
| 2-cell | | donor A | embryo 3 (1 | nd | nd | f | f | f | A/C | nd | |
| | | | cell) | | | | | | | | |
| | | | embryo 7 (4 of | | | | | | | | |
| | | | 7 cells) | | | | | | | | |
| 2-cell | | donor A | embryo 7 | nd | nd | C | A | C | C | nd | |
| 2-cell | | donor A | embryo 7 | nd | nd | T | G | T | A/A | nd | |
| 2-cell | | donor A | embryo 7 | nd | nd | T | G | T | A/C | nd | |
| 2-cell | | donor A | embryo 7 | nd | nd | T | G | T | A | nd | |
| | | | embryo 8 (7 of | | | | | | | | |
| | | | 8 cells) | | | | | | | | |
| 2-cell | | donor A | embryo 8 | normal | f | T | G | T | C | nd | |
| 2-cell | | donor A | embryo 8 | f | nd | T | G | T | A | A (100%) | |
| 2-cell | | donor A | embryo 8 | nd | nd | T | G | T | A | A (100%) | |
| 2-cell | | donor A | embryo 8 | nd | nd | T | G | T | A | nd | |
| 2-cell | | donor A | embryo 8 | nd | nd | T | G | T | A | nd | |
| 2-cell | | donor A | embryo 8 | nd | nd | T | G | T | A/C | nd | |
| 2-cell | | donor A | embryo 8 | nd | nd | T | G | T | A | nd | |
| | | | embryo (3 of 4 | | | | | | | | |
| | | | cells/fragments) | | | | | | | | |
| 2-cell | | donor A | embryo one | paternal 6p | A | T | G | T | f | na | |
| | | | | fragment | | | | | | | |
| | | | | only | | | | | | | |
| 2-cell | FIG. 5F | donor A | embryo one | del 6q (pat) | G/A | T | G | T | f | na | |
| 2-cell | FIG. 5F | donor A | embryo one | del 6p (pat) | G | C | A | C | A | A (100%) | |
| | | | | paternal, | | | | | | | |
| | | | | dup 6q | | | | | | | |
| | | | | (pat) | | | | | | | |
| | | | embryo (3 | | | | | | | | |
| | | | cells) | | | | | | | | |
| 2-cell | | donor A | embryo 2 | f | f | C | A | C | A | A (100%) | |
| 2-cell | FIG. 4E- | donor A | embryo 2 | monosomy | G | C | A | C | A | A (100%) | |
| | F | | | 6 (pat) | | | | | | | |
| 2-cell | FIG. 4E- | donor A | embryo 2 | del 6q (pat) | G/A | C/T | A/G | C/T | A | A (100%) | |
| | F | | | | | | | | | | |
| 2-cell | | donor A | morula multiple | nd | nd | T | G | T | A | A (99%) / C | |
| | | | cells embryo 10 | | | | | | | (1%) | |
| | | | biopsy 1 | | | | | | | | |
| 2-cell | | donor A | morula multiple | f | nd | T | G | T | A | A (100%) | |
| | | | cells embryo 10 | | | | | | | | |
| | | | biopsy 2 | | | | | | | | |
| 2-cell | | donor A | morula multiple | nd | nd | C/T | A/G | C/T | A | A (100%) | |
| | | | cells embryo 11 | | | | | | | | |
| na | | donor E | gDNA egg | nd | nd | T/T | G/G | T/T | A/C | na | |
| | | | donor E | | | | | | | | |
| 2-cell | | donor E | morula multiple | nd | nd | T | G | T | C/C | C (100%) | |
| | | | cells embryo 12 | | | | | | | | |
| 2-cell | | donor E | blastocyst | nd | nd | T | G | T | C/C | C (100%) | |
| | | | multiple cells | | | | | | | | |
| | | | embryo 13 | | | | | | | | |
| na | | donor H | gDNA egg | nd | nd | C/C | A/A | C/C | A/A | na | |
| | | | donor H | | | | | | | | |
| | | | embryo 5 (6 of | | | | | | | | |
| | | | 8 cells) | | | | | | | | |
| 2-cell | | donor H | embryo 5 | nd | nd | C | A | C | A | nd | |
| 2-cell | | donor H | embryo 5 | nd | nd | C/T | A/G | C/T | A | A (100%) | |
| 2-cell | | donor H | embryo 5 | nd | nd | C | A | C | A | nd | |
| 2-cell | | donor H | embryo 5 | nd | nd | C/T | A/G | C/T | A/C | nd | |
| 2-cell | | donor H | embryo 5 | nd | nd | T | G | T | A/C | nd | |
| 2-cell | | donor H | embryo 5 | nd | nd | nd | nd | nd | C | nd | |
| | | | embryo 6 (5 of | | | | | | | | |
| | | | 6 cells) | | | | | | | | |
| 2-cell | | donor H | embryo 6 | nd | nd | T | G | T | A/C | nd | |
| 2-cell | | donor H | embryo 6 | nd | nd | T | G | T | A/C | nd | |
| 2-cell | | donor H | embryo 6 | nd | nd | C/T | A/G | C/T | A | nd | |
| 2-cell | | donor H | embryo 6 | nd | nd | C | A | C | A | nd | |
| 2-cell | | donor H | embryo 6 | nd | nd | C | A | C | A/A | nd | |
| | | | embryo 9 (8 of | | | | | | | | |
| | | | 10 cells) | | | | | | | | |
| 2-cell | FIG. 4B | donor H | embryo 9 | nd | nd | C | A | C | A | nd | |
| 2-cell | | donor H | embryo 9 | nd | nd | C | A | C | A | nd | |
| 2-cell | FIG. 4B | donor H | embryo 9 | nd | nd | C/T | A/G | C/T | A/C | A (53%) / C | |
| | | | | | | | | | | (47%) | |
| 2-cell | FIG. 4B | donor H | embryo 9 | nd | nd | C/T | A/G | C/T | A | nd | |
| 2-cell | | donor H | embryo 9 | nd | nd | C/T | A/G | C/T | A | A (100%) | |
| 2-cell | | donor H | embryo 9 | nd | nd | C/T | A/G | C/T | A/C | nd | |
| 2-cell | | donor H | embryo 9 | nd | nd | T | G | T | C | C (100%) | |
| 2-cell | | donor H | embryo 9 | nd | nd | C | A | C | A | nd | |
| other events | | | | | | | | | | | |
| na | | na | gDNA sperm | normal | A/A | T/T | G/G | T/T | C/C | na | |
| | | | donor | | | | | | | | |
| na | | donor A | gDNA egg | normal | G/G | C/T | A/G | C/T | A/A | na | |
| | | | donor A | | | | | | | | |
| 2PN | | donor A | blastomere cell | normal | G/A | C/T | A/G | C/T | A/C | A(32%) / C | |
| | | | (2 cells) | | | | | | | (68%) | |
| 2PN | | donor A | sister | normal | f | nd | nd | nd | A | nd | |
| | | | blastomere cell | | | | | | | | |
| | | | (2 cells) | | | | | | | | |
| na | | donor G | gDNA egg | normal | nd | C/C | A/A | C/C | A/A | na | |
| | | | donor G | | | | | | | | |
| 2-cell | | donor G | blastomere | nd | A | T | G | T | C | nd | |
| androgenesis | | | androgenesis | | | | | | | | |
| | | rs758109813 | | | | | | | | | chr16 |
| | | MUTATION | rs758109813 | | | | | altered | | chr16 | karyotype |
| | rs758109813 | SITE deep | wt/fs allelic | | | | | allele | | off- | (* = off- |
| Cas9 RNP | MUTATION | sequencing | discrimination | rs12205397 | rs453084 | rs34809101 | rs6936438 | sequence (@ | | target | target |
| injection at | SITE | (FIG. 4C) | qPCR | (+834) | 1 (+931) | (+10.34 kb) | (+10.75 kb) | rs758109813) | remarks | indel | effect) |
| Androgenesis | | | | | | | | | | | |
| @ 20 h (no | | | | | | | | | | | |
| maternal | | | | | | | | | | | |
| genome) | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| MII | indel | nd | nd | A | A | nd | nd | CTT(− | MMEJ −5 bp | nd | nd |
| androgenesis | | | | | | | | 5 bp)CTG- | | | |
| | | | | | | | | AGG | | | |
| MII | f | nd | nd | f | f | f | f | na | failed | nd | nd |
| androgenesis | | | | | | | | | amplification | | |
| MII | fs | nd | nd | f | f | nd | nd | na | unmodified | nd | nd |
| androgenesis | | | | | | | | | | | |
| MII | indel/indel | nd | nd | A | A | nd | nd | CTGTAAC | MMEJ −5 bp | nd | nd |
| androgenesis | | | | | | | | TG- | & NHEJ RF | | |
| | | | | | | | | AGG/CTT(− | restored | | |
| | | | | | | | | 5 bp)CTG- | | | |
| | | | | | | | | AGG (SEQ | | | |
| | | | | | | | | ID NO: 4) | | | |
| MII | f | nd | nd | f | f | f | f | na | failed | nd | nd |
| androgenesis | | | | | | | | | amplification | | |
| MII | indel | nd | nd | A | A | nd | nd | GTA(- | NHEJ | nd | nd |
| androgenesis | | | | | | | | 1 bp)TG- | | | |
| | | | | | | | | AGG | | | |
| MII | indel/indel | nd | nd | A | A | nd | nd | CTGTA(- | NHEJ/MM | nd | nd |
| androgenesis | | | | | | | | 1 bp)TGAG | EJ −5 bp | | |
| | | | | | | | | G/CTT(− | | | |
| | | | | | | | | 5 bp)CTG- | | | |
| | | | | | | | | AGG (SEQ | | | |
| | | | | | | | | ID NO: 5) | | | |
| MII | indel | nd | nd | A | A | nd | nd | CTT(− | MMEJ −5 bp | nd | nd |
| androgenesis | | | | | | | | 5 bp)CTG- | | | |
| | | | | | | | | AGG | | | |
| control | | | | | | | | | | | |
| genotyping | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | wt/wt | nd | G/G | G/G | T/T | G/G | GTCTTTC | original | nd | nd |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| control — | wt/fs | nd | nd | nd | nd | T/(—) | G/G | na | genotyping | nd | nd |
| none | | | | | | | | | control | | |
| control — | wt/fs | nd | nd | G/A | G/A | T/(—) | G/G | na | genotyping | wt | nd |
| none | | | | | | | | | control | | |
| na | wt/wt | nd | wt/wt | G/G | G/G | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| control — | wt/fs | nd | nd | G/A | G/A | — | A | na | genotyping | nd | nd |
| none | | | | | | | | | control | | |
| na | wt/wt | nd | wt/wt | G/G | G/G | nd | nd | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| control — | wt/fs | nd | wt/fs | nd | nd | nd | nd | na | genotyping | nd | normal |
| none | | | | | | | | | control | | |
| control — | wt/fs | nd | nd | nd | nd | nd | nd | na | genotyping | nd | normal |
| none | | | | | | | | | control | | |
| control — | wt/fs | nd | nd | nd | nd | nd | nd | na | genotyping | nd | nd |
| none | | | | | | | | | control | | |
| control — | wt/fs | nd | nd | nd | nd | nd | nd | na | genotyping | nd | nd |
| none | | | | | | | | | control | | |
| 2PN analysis | | | | | | | | | | | |
| @ 20 h after | | | | | | | | | | | |
| MII | | | | | | | | | | | |
| injection | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | G/G | G/G | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt | wt | wt/not | G | G | T | G | no paternal | loss of | nd | normal |
| | | | detected | | | | | allele | paternal | | |
| | | | | | | | | | allele | | |
| MII | wt | wt | wt/not | G | G | T | G | no paternal | loss of | nd | f |
| | | | detected | | | | | allele | paternal | | |
| | | | | | | | | | allele | | |
| na | wt/wt | nd | wt/wt | G/A | G/A | nd | nd | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt/indel | nd | nd | nd | nd | T/— | G/A | CTT(− | MMEJ −5 bp | nd | nd |
| | | | | | | | | 5 bp)CTG- | | | |
| | | | | | | | | AGG | | | |
| 2PN | | | | | | | | | | | |
| individual | | | | | | | | | | | |
| pronuclei @ | | | | | | | | | | | |
| 20 h | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | G/A | G/A | nd | nd | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| mMII - 1 | wt | nd | nd | nd | nd | — | A | na | na | wt | normal |
| pMII - 1 | indel | nd | nd | nd | nd | — | A | CTT(− | MMEJ −5 bp | wt | normal |
| | | | | | | | | 5 bp)CTG- | | | |
| | | | | | | | | AGG | | | |
| mMII - 2 | wt | nd | nd | nd | nd | — | A | na | na | wt | nd |
| pMII - 2 | indel | nd | nd | nd | nd | — | A | CTT(− | MMEJ −5 bp | 5 bp | normal |
| | | | | | | | | 5 bp)CTG- | | del/8 | |
| | | | | | | | | AGG | | bp del | |
| mMII - 3 | wt | nd | nd | nd | nd | — | A | na | na | wt | normal |
| pMII - 3 | fs | nd | nd | nd | nd | — | A | na | unmodified | wt | normal |
| mMII - 4 | wt | nd | nd | nd | nd | — | A | na | na | wt | normal |
| pMII - 4 | indel | nd | nd | nd | nd | — | A | CTTTCTG | NHEJ | wt | normal |
| | | | | | | | | CACACAG | | | |
| | | | | | | | | AGAGACT | | | |
| | | | | | | | | G-AGG | | | |
| | | | | | | | | (SEQ ID | | | |
| | | | | | | | | NO: 6) | | | |
| mMII - 5 | wt | nd | nd | nd | nd | f | f | na | na | wt | normal |
| pMII - 5 | f | nd | nd | nd | nd | — | A | no paternal | no paternal | f | normal |
| | | | | | | | | allele | on target | | |
| | | | | | | | | | genotype | | |
| mMII - 6 | wt | nd | nd | nd | nd | — | nd | na | na | 5 bp | normal |
| | | | | | | | | | | del/8 | |
| | | | | | | | | | | bp del | |
| pMII - 6 | indel | nd | nd | nd | nd | — | nd | CTG (-3 bp) | NHEJ RF | 5 bp | normal |
| | | | | | | | | TG-AGG | restored | del/in | |
| | | | | | | | | | | del | |
| mMII - 7 | wt | nd | nd | nd | nd | nd | nd | na | na | wt | nd |
| pMII - 7 | f | nd | nd | nd | nd | — | A | no paternal | no paternal | f | normal |
| | | | | | | | | allele | on target | | |
| | | | | | | | | | genotype | | |
| mMII - 8 | wt | nd | nd | nd | nd | nd | A | na | na | 5 bp | normal |
| | | | | | | | | | | del/wt | |
| pMII - 8 | indel | nd | nd | nd | nd | nd | A | CTGTAAC | NHEJ, | 5 bp | normal |
| | | | | | | | | TG-AGG | reading | del/wt | |
| | | | | | | | | (SEQ ID | frame | | |
| | | | | | | | | NO: 7) | restored | | |
| Blastomere | | | | | | | | | | | |
| collection | | | | | | | | | | | |
| after MII | | | | | | | | | | | |
| injection | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | G/G | G/G | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt | wt (100%) | f | G | G | f | f | na | loss of | f | f |
| | | | | | | | | | paternal | | |
| | | | | | | | | | allele | | |
| MII | wt | wt (100%) | wt/not | G | G | T | T | na | loss of | 6 bp | normal |
| | | | detected | | | | | | paternal | ins/wt | |
| | | | | | | | | | allele | | |
| MII | wt | wt (100%) | f | f | f | T | T | na | loss of | 6 bp | normal |
| | | | | | | | | | paternal | ins/wt | |
| | | | | | | | | | allele | | |
| MII | wt | wt (100%) | wt/not | G | G | T/— | G/A | na | loss of | 6 bp | normal |
| | | | detected | | | | | | paternal | ins/wt | |
| | | | | | | | | | allele | | |
| MII | wt | wt (100%) | wt/not | G | G | T | T | na | loss of | nd | normal |
| | | | detected | | | | | | paternal | | |
| | | | | | | | | | allele | | |
| MII | wt | nd | nd | G | G | T | T | na | loss of | 6 bp | nd |
| | | | | | | | | | paternal | ins/wt | |
| | | | | | | | | | allele | | |
| na | wt/wt | nd | wt/wt | A/A | A/A | —/— | A/A | GTCTTTC | original | wt | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt/indel | A(67%) / | wt/not | A | A | — | A | TTTCTTCT | NHEJ | f | monosomy |
| | | edited | detected | | | | | (−1 bp)T(− | | | 16q |
| | | (33%) | | | | | | 1 bp)CTG- | | | (pat) |
| | | | | | | | | AGG (SEQ | | | FRA16F |
| | | | | | | | | ID NO: 8) | | | |
| MII | wt/indel | A(56%) / | wt/not | A | A | — | A | TTTCTTCT | NHEJ | 12 bp | dup |
| | | edited | detected | | | | | (−1 bp)T(− | | ins | 16q21-tel |
| | | (44%) | | | | | | 1 bp)CTG- | | | (pat) |
| | | | | | | | | AGG (SEQ | | | FRA16I |
| | | | | | | | | ID NO: 8) | | | |
| MII | wt/indel | A(45%) / | wt/not | A | A | — | A | TTTCTTCT | NHEJ | 12 bp | normal |
| | | edited | detected | | | | | (−1 bp)T(− | | ins/wt | |
| | | (55%) | | | | | | 1 bp)CTG- | | | |
| | | | | | | | | AGG (SEQ | | | |
| | | | | | | | | ID NO: 8) | | | |
| MII | wt/indel | A(80%) / | wt/not | A | A | — | A | TTTCTTCT | NHEJ | 5 bp | normal |
| | | edited | detected | | | | | (−1 bp)T(− | | ins/12 | |
| | | (20%) | | | | | | 1 bp)CTG- | | bp ins | |
| | | | | | | | | AGG (SEQ | | | |
| | | | | | | | | ID NO: | 8) | | |
| MII | wt | wt (100%) | wt/not | nd | nd | — | A | Na | loss of | 5 bp | monosomy |
| | | | detected | | | | | | paternal | ins | 16 (pat) |
| | | | | | | | | | allele | | |
| MII | wt/indel | A(92%) / | wt/not | nd | nd | — | A | CTT(− | MMEJ −5 bp | 5 bp | normal |
| | | edited | detected | | | | | 5 bp)CTG- | ins/wt | | |
| | | (8%) | | | | | | AGG | | | |
| MII | wt/indel | A(23%) / | wt/not | A | A | — | A | CTT(− | MMEJ −5 bp | 9 bp | normal |
| | | edited | detected | | | | | 5 bp)CTG- | ins/wt | | |
| | | (77%) | | | | | | AGG | | | |
| MII | wt/indel | A(32%) / | wt/not | A | A | — | A | CTT(− | MMEJ −5 bp | 9 bp | normal |
| | | edited | detected | | | | | 5 bp)CTG- | ins | | |
| | | (68%) | | | | | | AGG | | | |
| MII | wt/indel | A(79%) / | wt/not | A | A | — | A | CTT(− | MMEJ −5 bp | 8 bp | normal |
| | | edited | detected | | | | | 5 bp)CTG- | del | | |
| | | (21%) | | | | | | AGG | | | |
| MII | wt | nd | nd | nd | nd | nd | nd | na | loss of | nd | nd |
| | | | | | | | | | paternal | | |
| | | | | | | | | | allele | | |
| MII | wt | wt (100%) | wt/not | nd | nd | nd | nd | na | loss of | 6 bp | normal |
| | | | detected | | | | | | paternal | ins/wt | |
| | | | | | | | | | allele | | |
| MII | wt | wt (100%) | wt/not | nd | nd | nd | nd | na | loss of | 6 bp | del |
| | | | detected | | | | | | paternal | ins | 16q23.1- |
| | | | | | | | | | allele | | tel (mat)* |
| MII | f | na | no detection | nd | nd | f | f | no allele | loss of | 6 bp | normal |
| | | | | | | | | detected | paternal | ins | |
| | | | | | | | | | allele | | |
| MII | wt | wt (100%) | wt/not | nd | nd | nd | nd | na | loss of | 6 bp | Chr16 |
| | | | detected | | | | | | paternal | ins/54 | gain |
| | | | | | | | | | allele | bp | (mat), del |
| | | | | | | | | | | indel | 16q23.1- |
| | | | | | | | | | | | tel (mat)* |
| MII | f | na | no detection | f | f | f | f | no allele | loss of | 6 bp | dup |
| | | | | | | | | detected | paternal | ins | 16q23.1- |
| | | | | | | | | | allele | | tel (mat)* |
| MII | wt | wt (100%) | wt/not | A | A | nd | nd | na | loss of | nd | nullisomy |
| | | | detected | | | | | | paternal | | 16 |
| | | | | | | | | | allele | | |
| MII | indel | nd | failed | nd | nd | nd | nd | CTTTCTTC | loss of | 6 bp | del |
| | | | | | | | | T(− | paternal | ins | 16q23.1- |
| | | | | | | | | 9 bp)GTTTT | allele, | | tel (mat)* |
| | | | | | | | | (SEQ ID | altered | | |
| | | | | | | | | NO: 9) | maternal | | |
| | | | | | | | | | allele by | | |
| | | | | | | | | | MMEJ | | |
| Trophectoderm | | | | | | | | | | | |
| Biopsy | | | | | | | | | | | |
| after MII | | | | | | | | | | | |
| injection | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | A/A | A/A | —/— | A/A | GTCTTTC | original | wt | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt/indel | nd | wt/not | nd | nd | — | A | CTGTGTC | NHEJ, two | wt | normal |
| | | | detected | | | | | TTACTG- | concordant | | |
| | | | | | | | | AGG (SEQ | biopsies | | |
| | | | | | | | | ID NO: 10) | | | |
| MII | wt | wt (100%) | wt/not | nd | nd | — | A | na | loss of | 2 bp | normal |
| | | | detected | | | | | | paternal | del/wt | |
| | | | | | | | | | allele | | |
| MII | wt | wt (100%) | wt/not | nd | nd | — | A | na | loss of | 8 bp | normal |
| | | | detected | | | | | | paternal | del/wt | |
| | | | | | | | | | allele, two | | |
| | | | | | | | | | concordant | | |
| | | | | | | | | | biopsies | | |
| TE biopsies | | | | | | | | | | | |
| without | | | | | | | | | | | |
| Sanger | | | | | | | | | | | |
| genotype | | | | | | | | | | | |
| MII | nd | nd | wt/not | nd | nd | nd | nd | nd | altered | nd | normal |
| | | | detected | | | | | | paternal | | |
| | | | | | | | | | allele, | | |
| | | | | | | | | | concordant | | |
| | | | | | | | | | with ICM | | |
| MII | nd | nd | wt/not | nd | nd | nd | nd | nd | altered | nd | normal |
| | | | detected | | | | | | paternal | | |
| | | | | | | | | | allele, | | |
| | | | | | | | | | defined | | |
| | | | | | | | | | paternal | | |
| | | | | | | | | | genotype | | |
| | | | | | | | | | not | | |
| | | | | | | | | | available | | |
| MII | nd | nd | wt/not | nd | nd | nd | nd | nd | altered | nd | normal |
| | | | detected | | | | | | paternal | | |
| | | | | | | | | | allele, | | |
| | | | | | | | | | concordant | | |
| | | | | | | | | | with ICM | | |
| MII | nd | nd | wt/not | nd | nd | nd | nd | nd | altered | nd | normal |
| | | | detected | | | | | | paternal | | |
| | | | | | | | | | allele, | | |
| | | | | | | | | | concordant | | |
| | | | | | | | | | with ICM | | |
| ESC | | | | | | | | | | | |
| derivation | | | | | | | | | | | |
| after MII | | | | | | | | | | | |
| injection | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | G/G | G/G | nd | nd | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt/indel | nd | wt/not | G/A | G/A | T/- | G/A | CTGTTTTT | NHEJ | 14 bp | normal |
| | | | detected | | | | | CTTCTG- | ins/wt | | |
| | | | | | | | | AGG (SEQ | | | |
| | | | | | | | | ID NO: 11) | | | |
| MII | wt/indel | nd | wt/not | G/A | G/A | T/— | G/A | CTGTCTT | MMEJ plus | 1 bp | normal |
| | | | detected | | | | | CTGTGTC | l1 bp | del/wt | |
| | | | | | | | | TGTCTTT- | insertion | | |
| | | | | | | | | 8 bpCTG- | | | |
| | | | | | | | | AGG (SEQ | | | |
| | | | | | | | | ID NO: 12) | | | |
| MII | wt/fs | wt/fs | wt/fs | G/A | G/A | T/— | G/A | na | no change | nd | normal |
| MII | wt/indel | nd | wt/not | nd | nd | nd | nd | CTGAAAC | NHEJ | wt | normal |
| | | | detected | | | | | TG-AGG | | | |
| | | | | | | | | (SEQ ID | | | |
| | | | | | | | | NO: 13) | | | |
| na | wt/wt | nd | wt/wt | nd | nd | —/— | A | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| MII | wt/indel | nd | nd | nd | nd | nd | nd | CTGTGTC | NHEJ | wt | nd |
| | | | | | | | | TTACTG- | | | |
| | | | | | | | | AGG (SEQ | | | |
| | | | | | | | | ID NO: 14) | | | |
| na | wt/wt | nd | nd | nd | nd | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| control — | wt/fs | nd | nd | nd | nd | T/— | G/A | na | genotyping | nd | nd |
| none | | | | | | | | | control | | |
| 2-cell stage | | | | | | | | | | | |
| injections | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | wt/wt | wt/wt | G/G | G/G | T/T | G/G | GTCTTTC | original | nd | nd |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2-cell | wt | nd | nd | G | G | T | G | na | loss of | nd | nd |
| | | | | | | | | | paternal | | |
| | | | | | | | | | allele | | |
| 2-cell | wt/ indel | nd | nd | G/G | G/G | T | G | CTTCTGT | loss of | nd | nd |
| | | | | | | | | A [symbol] CTGGA | paternal | | |
| | | | | | | | | GG (SEQ | allele, | | |
| | | | | | | | | ID NO: 15) | altered | | |
| | | | | | | | | | maternal | | |
| | | | | | | | | | allele NHEJ | | |
| 2-cell | wt/indel | nd | nd | G/A | G/A | T | G | GTA(- | NHEJ | nd | nd |
| | | | | | | | | 1 bp)TG- | | | |
| | | | | | | | | AGG | | | |
| na | wt/wt | nd | wt/wt | G/G | G/G | TIT | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2-cell | wt/fs | nd | nd | nd | nd | nd | nd | na | no change | nd | nd |
| 2-cell | wt/indel | nd | nd | nd | nd | T | G | GTAACT(− | NHEJ | nd | nd |
| | | | | | | | | 1 bp)AGG | | | |
| 2-cell | wt/ indel | nd | nd | G/G | G/G | T | G | CTG(- | loss of | nd | nd |
| | | | | | | | | 1 bp)ACTG | paternal | | |
| | | | | | | | | GAGG | allele, | | |
| | | | | | | | | (SEQ ID | altered | | |
| | | | | | | | | NO: 16) | maternal | | |
| | | | | | | | | | allele NHEJ | | |
| 2-cell | wt/indel | nd | nd | G/A | G/A | T | G | CTTC(- | NHEJ | nd | nd |
| | | | | | | | | 4 bp)CTG- | | | |
| | | | | | | | | AGG (SEQ | | | |
| | | | | | | | | ID NO: 17) | | | |
| 2-cell | wt | nd | nd | G | G | T | G | na | loss of | nd | nd |
| | | | | | | | | | paternal | | |
| | | | | | | | | | allele | | |
| 2-cell | wt | nd | nd | A | A | nd | nd | na | possible IH- | nd | normal |
| | | | | | | | | | HR | | |
| 2-cell | wt | wt (100%) | nd | G | G | T | G | na | loss of | nd | f |
| | | | | | | | | | paternal | | |
| | | | | | | | | | allele | | |
| 2-cell | wt | wt (100%) | nd | nd | nd | T | G | na | loss of | nd | nd |
| | | | | | | | | | paternal | | |
| | | | | | | | | | allele | | |
| 2-cell | wt | nd | nd | nd | nd | nd | nd | na | loss of | nd | nd |
| | | | | | | | | | paternal | | |
| | | | | | | | | | allele | | |
| 2-cell | wt | nd | nd | G | G | nd | nd | na | loss of | nd | nd |
| | | | | | | | | | paternal | | |
| | | | | | | | | | allele | | |
| 2-cell | wt/indel | nd | nd | G/A | G/A | T/— | G/A | TCTGT(− | NHEJ | nd | nd |
| | | | | | | | | 1 bp)CTG- | | | |
| | | | | | | | | AGG (SEQ | | | |
| | | | | | | | | ID NO: 18) | | | |
| 2-cell | wt | nd | nd | G | G | T | G | na | loss of | nd | nd |
| | | | | | | | | | paternal | | |
| | | | | | | | | | allele | | |
| 2-cell | f | na | no detection | f | f | f | f | no allele | cleaved | f | nullisomic |
| | | | | | | | | detected | chromosome | | |
| | | | | | | | | | fragment | | |
| 2-cell | f | na | no detection | f | f | T | G | no allele | no allele | 5 bp | del |
| | | | | | | | | detected | detected | del | 16q23.1- |
| | | | | | | | | | | | tel (mat)* |
| 2-cell | wt | wt (100%) | wt/not | G | G | T | G | na | loss of | 1 bp | del |
| | | | detected | | | | | | paternal | del | 16q23.1- |
| | | | | | | | | | allele | | tel (pat)* |
| 2-cell | wt | wt (100%) | wt/not | nd | nd | T | G | na | loss of | nd | monosomy |
| | | | detected | | | | | | paternal | | 16 |
| | | | | | | | | | allele | | (mat) |
| 2-cell | wt | wt (100%) | wt/not | G | G | T | G | na | loss of | nd | normal |
| | | | detected | | | | | | paternal | | |
| | | | | | | | | | allele | | |
| 2-cell | wt | wt (100%) | wt/not | G | G | T | G | na | telomeric | nd | monosomy |
| | | | detected | | | | | | loss of | | 16 (pat) |
| | | | | | | | | | paternal | | |
| | | | | | | | | | allele | | |
| 2-cell | wt | wt (99%) / | na | nd | nd | T | G | AGAAAGA | MMEJ -8 bp | nd | nd |
| | (mosaic) | edited | | | | | | TCTTTCT(− | | | |
| | | (1%) | | | | | | 8 bp)G-AGG | | | |
| | | | | | | | | (SEQ ID | | | |
| | | | | | | | | NO: 19) | | | |
| 2-cell | wt | wt (100%) | na | nd | nd | nd | nd | na | loss of | nd | f |
| | | | | | | | | | paternal | | |
| | | | | | | | | | allele | | |
| 2-cell | wt | wt (100%) | na | nd | nd | T/— | G/A | na | loss of | nd | nd |
| | | | | | | | | | paternal | | |
| | | | | | | | | | allele | | |
| na | wt/wt | nd | nd | G/A | G/A | — | A/A | GTCTTTC | original | nd | nd |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2-cell | wt/indel | wt (33%) / | nd | nd | nd | — | A | CTT(− | MMEJ −5 bp | nd | nd |
| | | edited | | | | | | 5 bp)CTG- | | | |
| | | (67%) | | | | | | AGG | | | |
| 2-cell | wt/indel | wt (37%) / | nd | nd | nd | — | A | CTT(− | MMEJ −5 bp | nd | nd |
| | | edited | | | | | | 5 bp)CTG- | | | |
| | | (63%) | | | | | | AGG | | | |
| na | wt/wt | nd | nd | G/G | T/T | T/T | G/G | GTCTTTC | original | nd | nd |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2-cell | wt | nd | nd | G | G | T | G | na | loss of | nd | nd |
| | | | | | | | | | paternal | | |
| | | | | | | | | | allele | | |
| 2-cell | wt | wt (100%) | nd | G | G | T/— | G/A | na | loss of | nd | nd |
| | | | | | | | | | paternal | | |
| | | | | | | | | | allele | | |
| 2-cell | wt | nd | nd | G | G | T | G | na | loss of | nd | nd |
| | | | | | | | | | paternal | | |
| | | | | | | | | | allele | | |
| 2-cell | wt/fs | nd | nd | f | f | T/— | G/A | na | no change | nd | nd |
| 2-cell | wt/fs | nd | nd | G/A | G/A | T/— | G/A | na | no change | nd | nd |
| 2-cell | wt | nd | nd | A | A | T/— | G/A | na | possible IH- | nd | nd |
| | | | | | | | | | HR | | |
| 2-cell | wt/indel | nd | nd | A | A | T/— | G/A | CTTC(- | NHEJ | nd | nd |
| | | | | | | | | 4 bp)CTG- | | | |
| | | | | | | | | AGG (SEQ | | | |
| | | | | | | | | ID NO: 17) | | | |
| 2-cell | wt/indel | nd | nd | G/A | G/A | T/— | G/A | CTTC(- | NHEJ | nd | nd |
| | | | | | | | | 4 bp)CTG- | | | |
| | | | | | | | | AGG (SEQ | | | |
| | | | | | | | | ID NO: 17) | | | |
| 2-cell | wt | nd | nd | G | G | T/— | G/A | na | loss of | nd | nd |
| | | | | | | | | | paternal | | |
| | | | | | | | | | allele | | |
| 2-cell | wt | nd | nd | G | G | T | G | na | loss of | nd | nd |
| | | | | | | | | | paternal | | |
| | | | | | | | | | allele | | |
| 2-cell | wt/indel | nd | nd | G/G | G/G | T | G | CTGT [symbol] AC | loss of | nd | nd |
| | | | | | | | | TGGAGG | paternal | | |
| | | | | | | | | (SEQ ID | allele, wt | | |
| | | | | | | | | NO: 20) | maternal/ | | |
| | | | | | | | | | altered | | |
| | | | | | | | | | maternal | | |
| | | | | | | | | | allele NHEJ | | |
| 2-cell | wt | nd | nd | G | G | T | G | na | loss of | nd | nd |
| | | | | | | | | | paternal | | |
| | | | | | | | | | allele | | |
| 2-cell | wt | nd | nd | G | G | T/— | G/A | na | loss of | nd | nd |
| | | | | | | | | | paternal | | |
| | | | | | | | | | allele | | |
| 2-cell | wt/indel | wt (53%) / | nd | G/A | G/A | T/— | G/A | AGAAAGA | MMEJ -8 bp | nd | nd |
| | | edited | | | | | | TCTTTCT(− | | | |
| | | (47%) | | | | | | 8 bp)G-AGG | | | |
| | | | | | | | | (SEQ ID | | | |
| | | | | | | | | NO: 21) | | | |
| 2-cell | wt/indel | nd | nd | G/A | A | T/— | G/A | CTT(− | MMEJ −5 bp | nd | nd |
| | | | | | | | | 5 bp)CTG- | | | |
| | | | | | | | | AGG | | | |
| 2-cell | wt | wt (100%) | nd | G | G | T | G | na | telomeric | nd | nd |
| | | | | | | | | | loss of | | |
| | | | | | | | | | paternal | | |
| | | | | | | | | | allele | | |
| 2-cell | wt/indel | nd | nd | G/A | G/A | nd | G/A | CTT(− | MMEJ −5 bp | nd | nd |
| | | | | | | | | 5 bp)CTG- | | | |
| | | | | | | | | AGG | | | |
| 2-cell | wt | wt (100%) | nd | A | A | nd | nd | na | possible IH- | nd | nd |
| | | | | | | | | | HR | | |
| 2-cell | wt | nd | nd | G | G | T | G | na | loss of | nd | nd |
| | | | | | | | | | paternal | | |
| | | | | | | | | | allele | | |
| other events | | | | | | | | | | | |
| na | fs/fs | fs/fs | fs/fs | A/A | A/A | —/— | A/A | GTCTTTC | original | wt | normal |
| | | | | | | | | TTCTGTA | paternal | | |
| | | | | | | | | CTG-AGG | allele | | |
| | | | | | | | | (SEQ ID | | | |
| | | | | | | | | NO: 2) | | | |
| na | wt/wt | nd | wt/wt | G/G | G/G | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2PN | wt/indel | wt (33%) / | wt/not | G/A | G/A | T/— | G/A | TCTTCTG | NHEJ | nd | no call |
| | | edited | detected | | | | | T(− | | | |
| | | (67%) | | | | | | 1 bp)CTG- | | | |
| | | | | | | | | AGG (SEQ | | | |
| | | | | | | | | ID NO: 22) | | | |
| 2PN | wt | nd | wt/not | nd | nd | nd | nd | na | loss of | nd | del |
| | | | detected | | | | | | paternal | | 16q23.1- |
| | | | | | | | | | allele | | tel (pat)* |
| na | wt/wt | nd | nd | G/G | G/G | T/T | G/G | GTCTTTC | original | nd | normal |
| | | | | | | | | TTCTGTA | maternal | | |
| | | | | | | | | CTGGAGG | allele | | |
| | | | | | | | | (SEQ ID | | | |
| | | | | | | | | NO: 3) | | | |
| 2-cell | indel | nd | nd | A | A | T/— | G/A | CTT(− | MMEJ −5 bp | nd | nd |
| androgenesis | | | | | | | | 5 bp)CTG- | androgenesis | | |
| | | | | | | | | AGG | | | |

### Example 10—Chromosome Removal in Trisomy Embryo by CRISPR/Cas9 DSB

### Materials and Methods

### Human Embryos

Cryopreserved human 2 pronuclear (2PN) embryos were anonymously donated from couples who provided informed consent for use in research. Embryos were cryopreserved between the years 2007 and 2012 using One Step (PB1. PG, 1 M sucrose) cryopreservation solution, Sage embryo freeze kit or Quinn's embryo freeze kit. Embryos were stored in liquid nitrogen until use. For the experimental procedures, embryos were thawed then exposed to experimental conditions as outlined below. All experiments were conducted during incubation at 37° C. with 5% CO₂ and 20% 02. All human embryos were cultured for no more than 1-6 days, in accordance with at the time of conduct of the research internationally accepted standards to limit progression to less than 14 days (Adikusuma et al., 2017).

### RNP Preparation

2 nmol of single guide RNAs were obtained for Integrated DNA Technologies (IDT) with gRNA with the sequences listed in Table 3 and dissolved in 20 μL to a concentration of 100 μM sgRNA. For ribonucleoprotein (RNP) preparation, 3 μL of injection buffer, 2 μL of 63 μM IDT nlsCas9 v3, and 1.5 μL of 100 μM sgRNA were combined and incubated at room temperature for 5 minutes. Thereafter, 96.5 μL of an injection buffer was added. Injection buffer consists of 5 mM Tris-HCl, 0.1 mM EDTA, pH 7.8. The RNP solution was then centrifuged at 16000 RCF for 2 minutes prior to loading into the injection needle and cytoplasmic injection.

TABLE 3
gRNA Sequences

| gRNA | gRNA | | |
|---|---|---|---|
| Chromosome | Chromosome | gRNA | |
| position (hg38) | position (hg19) | chromosome arm | gRNA sequence |
| Chr16: | na | Chr16 centromere | 5′ATATTTGTATGTTAGAATC |
| 36221387 | | | TGGG (SEQ ID NO: 28) |
| Chr16: | chr16: 46489334 | Chr16 q arm | 5′AAGAATGGTACACTCGAG |
| 46455422 | | | GGAGG (SEQ ID NO: 29) |
| Chr16: | chr16: 33721043 | Chr16 p arm | 5′GGCGTTCAAGGACTTATT |
| 33918576** | | | CAAGG |
| | | | (r52659747)(SEQ ID NO: 30) |
| Chr16: | chr16: 34360916 | Chr16 p arm | 5′GACCGATGTATTCCACCT |
| 35126545 | | | ACAGG (SEQ ID NO: 31) |

### Embryo Manipulations

Embryo manipulations were performed in an inverted Olympus IX71 microscope using Narishige micromanipulators on a stage heated to 37° C. Frozen 2PN (day 1) embryos were thawed using One Step or Sage Embryo thaw kit and pronucleus formation was confirmed. Embryo polar bodies were collected whenever possible. RNP was prepared as above. The tip of an injection needle was nicked and small, but visible amounts of the Cas9RNP was injected manually into the cytoplasm of thawed embryos using a Narishige micromanipulator. Embryos were then cultured in Global Total (Cooper Surgical) in an incubator (Thermo Scientific, Heracell 150i) at 6% CO₂, 37° C. until collection.

### Genome Amplification and Genotyping

Single blastomeres were collected on day 2 to day 3, or if indicated, on day 4, on tie heated stage of an inverted Olympus IX71 microscope equipped with Narishige micromanipulators and a zona pellucida laser (Hamilton-Thorne). Trophectoderm biopsies were obtained on day 6 of development using 300 ms laser pulses to separate trophectoderm from the inner cell mass. All samples were placed in single tubes with 2 or 4 μL of PBS. Amplification was performed using REPLI-g single kit (Qiagen) according to manufacturer's instructions, using either a half reaction for 2 μL, or a full reaction for 4 μL.

Genotyping was performed using primers for amplification and sequencing. PCR was performed using AmpliTaq Cold. PCR products were run on a 1.5% agarose gel for visual inspection of product size and submitted to Genewiz for Sanger sequencing. Base changes were analyzed at the region of Cas9 target sites using Snap Gene2 and ICE analysis (Synthego).

### Genome-Wide SNP Array

Embryo biopsies were amplified at Columbia University using REPLI-g single cell kit. according to manufacturer's instructions. Copy number and genotyping analysis was performed using gSUITE software (Genomic Prediction). For copy number analysis, raw intensities from Affymetrix Axiom array are first processed according to the method described (Mayrhofer et al., 2016). After normalizing with a panel of normal males and females, the copy number is then calculated for each probeset. Normalized intensity is displayed. Mapping of endogenous fragile sites was done through visual evaluation of loss of heterozygosity. Break points were mapped to chromosomal bands by visual analysis of SNP array chromosome plots including analysis of both copy number signal and heterozygosity calls. The accuracy of mapping is between 100-500 kb. A segmental error was defined as the gain or loss of a chromosome arm or segment. Fragmented chromosome was called if there were multiple break points within a given chromosome. Chromosomal coordinates were mapped using probe intensity data on samples where chromosomal changes included nullisomy or a difference of at least two copies.

### Quantification and Statistical Analysis

Statistical analysis was performed using Fisher's exact test as indicated. A p-value of less than 0.05 was considered significant.

### Results

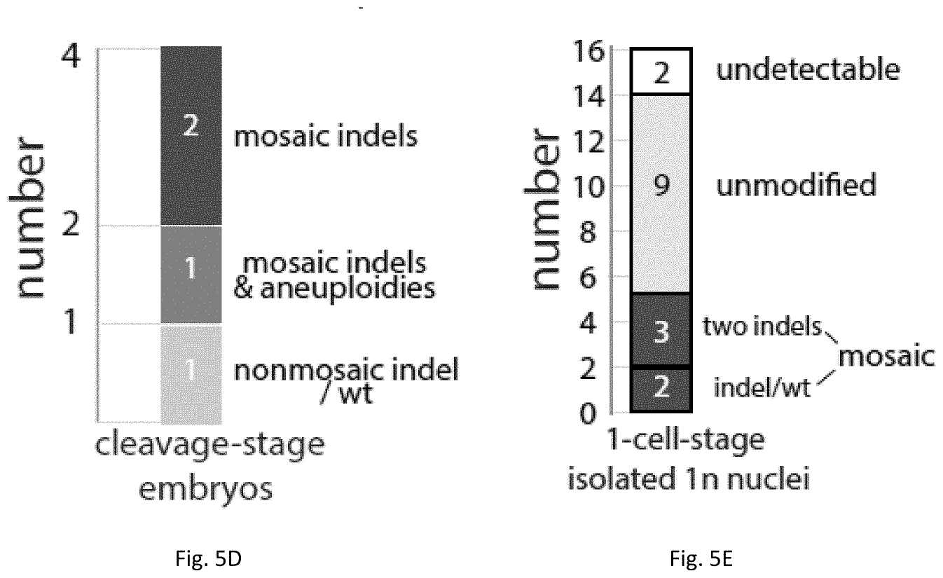

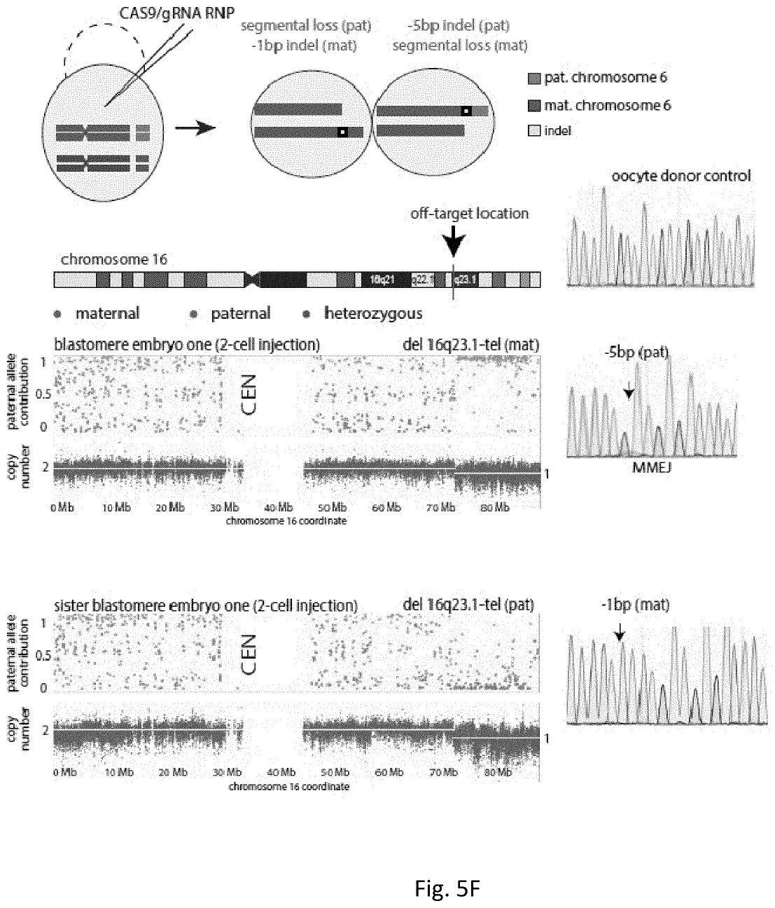

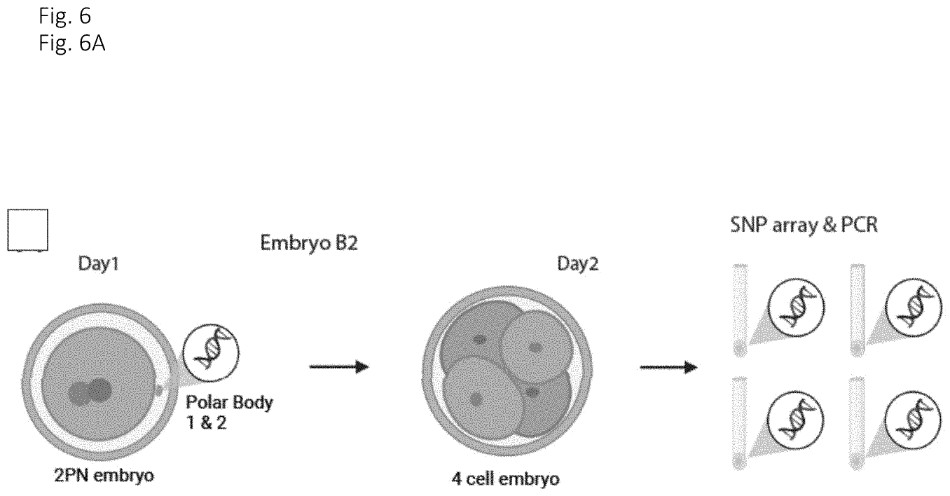

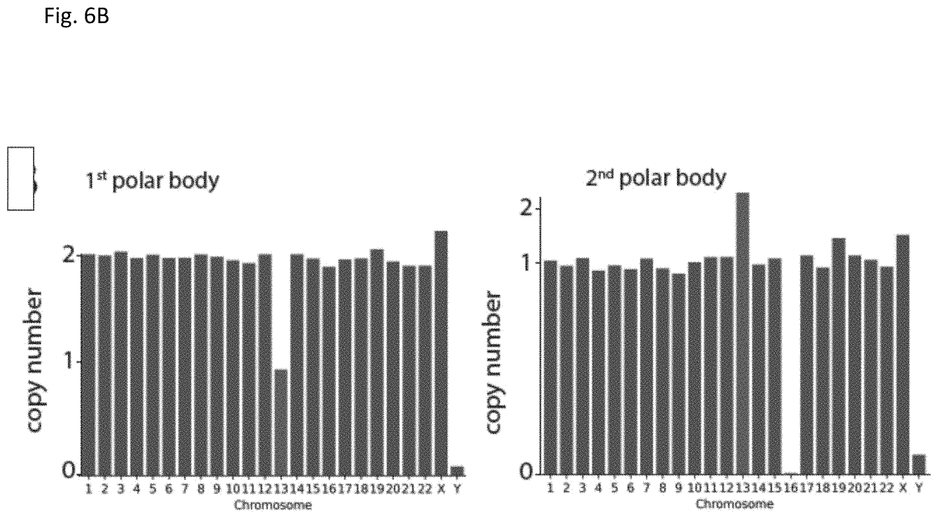

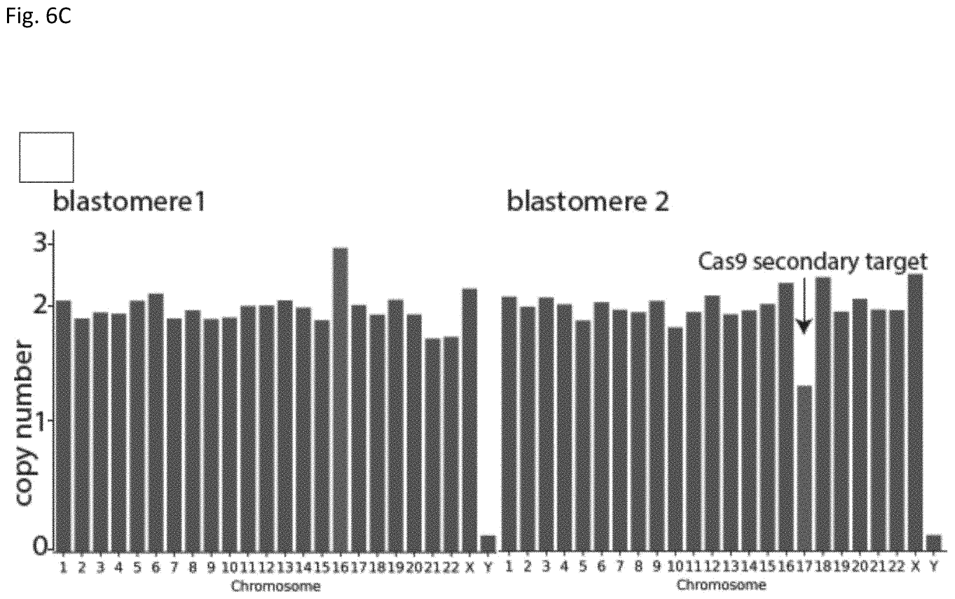

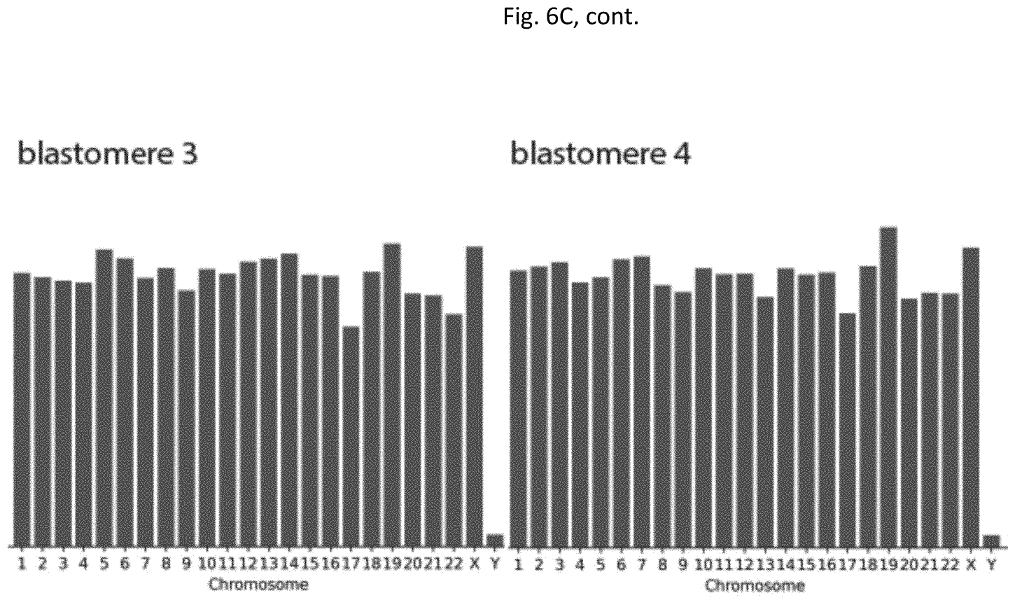

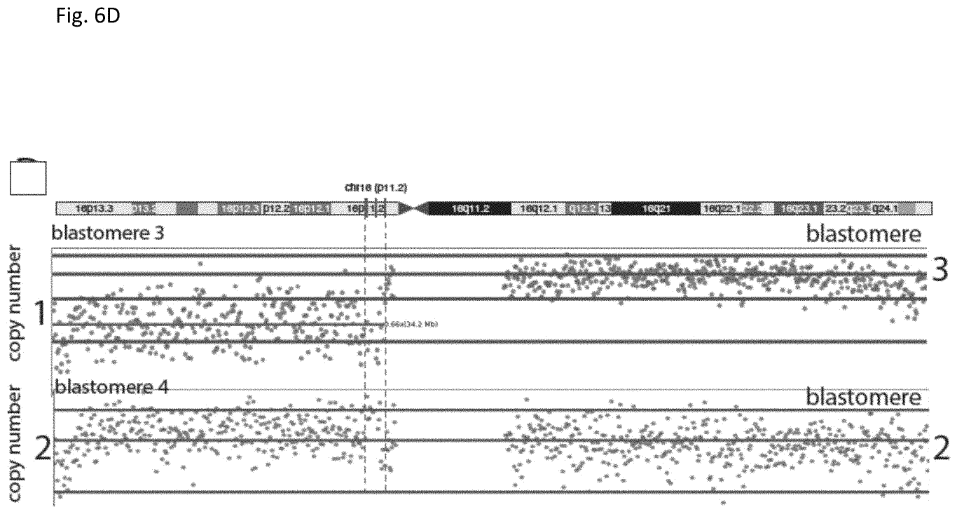

In this experiment, polar bodies of 10 embryos were collected at the time of CRISPR/Cas9 injection and successfully analyzed by SNP array to identify maternal meiotic errors (FIG. 6A). The polar body from one embryo (B2) showed a nullisomy for chromosome 16 (FIG. 6B). This maternal meiotic error is expected to result in a trisomy 16 in all embryonic cells. Interestingly, the same embryo showed reciprocal segregation errors of chromosome 13: the first polar body showed a loss in chromosome 13 genomic DNA and the second polar body showed a gain in chromosome 13 genomic DNA (FIG. 6B). This resulted in a normal chromosome 13 complement in the embryo (FIG. 6C), a spontaneous correction similar to what has previously been observed. Thus, information of both polar bodies is required to infer maternal aneuploidies in the fertilized zygote.

This embryo (B2) was injected with CRISPR/Cas9 targeting the pericentromeric p arm of chromosome 16 at the pronuclear stage (SEQ ID NO: 30). The embryo was then cultured to day 2 at the 4-cell stage. All 4 cells were analyzed with SNP array, which demonstrated copy number changes for chromosome 16, and PCR analysis revealed indel formation at the targeted sites. One cell demonstrated, as expected, 3 copies of chromosome 16. Two cells showed a numerical imbalance of 16p and 16q arm (FIG. 6D, TOP). The remaining fourth cell showed 2 balanced copies of chromosome 16 (FIG. 6D, BOTTOM). This embryo provides evidence that CRIPSR/Cas9 can remove a trisomy in the preimplantation human embryo. However, chromosomal changes were also observed on chromosome 17, as a perfect secondary gRNA target sequence is also found at chr17q12 (FIG. 2C). No further development of the embryo was tested.

Previous studies have shown that the use of multiple guide RNAs could result in frequent Y chromosome loss in mouse embryonic stem cells and zygotes (Adikusuma et al., 2017). To determine whether multiple gRNA targeting one chromosome resulted in more chromosome loss, 5 embryos were injected with 3 guide RNAs as set forth in Table 3 targeting chromosome 16 at 3 different sites: the pericentromeric p arm, centromere and pericentromeric q arm of Chromosome 16. For comparison, 10 embryos were injected with gRNA targeting a single site only at the pericentromeric p or q region of Chromosome 16. When specifically looking at the rates of chromosome loss, they were similar between embryos injected with one vs. three gRNAs (1 gRNA: 43% [31/72] vs 3 gRNA: 43% [25/58]). When considering all chromosomal changes, including losses and gains on chromosome 16, 1 single gRNA site resulted in 66% (24/36) of blastomeres with chromosomal 16 copy number changes. Three separate gRNAs on chromosome 16, of which one gRNA was affected by a common SNP, resulted in 86% (25/29) of blastomeres with chromosomal 16 copy number changes.

These results show that chromosome loss can be increased when several gRNAs are combined. Chromosome removal may be increased further than with 3 gRNAs, as the gRNA targeting the centromeric site had a common SNP close to the PAM site that affected gRNA function and also prevented the formation of indels.

### REFERENCES

Abd El-Aziz, et al. (2008). EYS, encoding an ortholog of Drosophila spacemaker, is mutated in autosomal recessive retinitis pigmentosa. Nat Genet 40, 1285-1287.
Adikusuma, et al. (2018). Large deletions induced by Cas9 cleavage. Nature 560, E8-e9.
Adikusuma, et al. (2017). Targeted Deletion of an Entire Chromosome Using CRISPR/Cas9. Molecular therapy: the journal of the American Society of Gene Therapy 25, 1736-1738.
Egli, et al. (2018). Inter-homologue repair in fertilized human eggs? Nature 560, E5-e7.
Fogarty, et al. (2017). Genome editing reveals a role for OCT4 in human embryogenesis. Nature 550, 67-73.
Franasiak, et al. (2014). Aneuploidy across individual chromosomes at the embryonic level in trophectoderm biopsies: changes with patient age and chromosome structure. Journal of assisted reproduction and genetics 31, 1501-1509.
Hassold and Hunt (2001). To err (meiotically) is human: the genesis of human aneuploidy. Nat Rev Genet 2, 280-291.
Jasin (2018). Under the Influence: Cas9 Ends and DNA Repair Outcomes. Crispr j 1, 132-134.
Jasin and Rothstein (2013). Repair of strand breaks by homologous recombination. Cold Spring Harb Perspect Biol 5, a012740.
Khan, et al. (2010). Missense mutations at homologous positions in the fourth and fifth laminin A G-like domains of eyes shut homolog cause autosomal recessive retinitis pigmentosa. Mol Vis 16, 2753-2759.
Kosicki, et al. (2018). Repair of double-strand breaks induced by CRISPR-Cas9 leads to large deletions and complex rearrangements. Nature Biotechnology.
Lea and Niakan (2019). Human germline genome editing. Nat Cell Biol 21, 1479-1489.
Lemos, et al. (2018). CRISPR/Cas9 cleavages in budding yeast reveal templated insertions and strand-specific insertion/deletion profiles. Proc Natl Acad Sci USA 115, E2040-e2047.
Liang, et al. (2015). CRISPR/Cas9-mediated gene editing in human tripronuclear zygotes. Protein Cell 6, 363-372.
Ma, et al. (2018). Ma et al. reply. Nature 560, E10-E23.
Ma, et al. (2017). Correction of a pathogenic gene mutation in human embryos. Nature 548, 413-419.
Mayrhofer. et al. 2016. Rawcopy: Improved copy number analysis with Affymetrix arrays. Sci Rep, 6, 36158.
Mrasek et al. 2010. Global screening and extended nomenclature for 230 aphidicolin-inducible fragile sites, including 61 yet unreported ones. Int J Oncol, 36, 929-40.
McCoy, et al. (2015). Evidence of Selection against Complex Mitotic-Origin Aneuploidy during Preimplantation Development. PLoS Genet 11, e1005601.
Reichmann, et al. (2018). Dual-spindle formation in zygotes keeps parental genomes apart in early mammalian embryos. Science 361, 189-193.
Sfeir and Symington (2015). Microhomology-Mediated End Joining: A Back-up Survival Mechanism or Dedicated Pathway? Trends Biochem Sci 40, 701-714.
Treff, et al. (2019). Validation of concurrent preimplantation genetic testing for polygenic and monogenic disorders, structural rearrangements, and whole and segmental chromosome aneuploidy with a single universal platform. Eur J Med Genet 62, 103647.
Vanneste, et al. (2009). Chromosome instability is common in human cleavage-stage embryos. Nat Med 15, 577-583.
Yamada, et al. (2014). Human oocytes reprogram adult somatic nuclei of a type 1 diabetic to diploid pluripotent stem cells. Nature 510, 533-536.
Zakarin, et al. (2018). Compensating human subjects providing oocytes for stem cell research: 9-year experience and outcomes. J Assist Reprod Genet.
Zuo, et al. (2017). CRISPR/Cas9-mediated targeted chromosome elimination. Genome biology 18, 224-224.

1. A method of correcting an aneuploidy in an embryo comprising introducing into the embryo at least one guide RNA or DNA encoding at least one guide RNA, wherein the at least one guide RNA targets a single nucleotide polymorphism flanking the centromere of an extra chromosome; and an RNA-guided endonuclease, or DNA encoding an RNA-guided endonuclease, wherein the endonuclease introduces a single double-stranded break in a targeted site resulting in the loss or elimination of the extra chromosome.

2. The method of claim 1, wherein more than one guide RNA or DNA encoding the guide RNA is introduced into the embryo, wherein a first at least one guide RNA or DNA encoding the guide RNA targets a single nucleotide polymorphism flanking one side of the centromere of the extra chromosome and a second at least one guide RNA or DNA encoding the guide RNA targets a single nucleotide polymorphism flanking an opposite side of the centromere of the extra chromosome.

3. The method of claim 2, wherein two to eight guide RNAs or DNA encoding the guide RNAs are introduced into the embryos, wherein at least one guide RNA or DNA encoding the guide RNA targets a single nucleotide polymorphism flanking one side of the centromere of the extra chromosome and at least one guide RNA or DNA encoding the guide RNA targets a single nucleotide polymorphism flanking an opposite side of the centromere of the extra chromosome.

4. The method of claim 1, wherein the single nucleotide polymorphism flanking the centromere is within about 1 to about 5 Mb from the centromere.

5. (canceled)

6. The method of claim 1, wherein the RNA-guided endonuclease is a Cas nuclease.

7. (canceled)

8. The method of claim 1, wherein the at least one guide RNA and the RNA-guided endonuclease are introduced to the embryo in a ribonucleoprotein complex.

9. (canceled)

10. (canceled)

11. The method of claim 1, wherein the aneuploidy is chosen from the group consisting of trisomy 8 (Warnany Syndrome), trisomy 9, trisomy 13 (Patau syndrome), trisomy 16, trisomy 18 (Edwards syndrome), trisomy 21 (Down syndrome), trisomy 22, trisomy X (Klinefelter syndrome) and trisomy Y (Jacob syndrome.

12. The method of claim 1, further comprising performing preimplantation genetic screening of the embryo prior to the introduction of the at least one guide RNA or DNA encoding at least one guide RNA, and the RNA-guided endonuclease, or DNA encoding an RNA-guided endonuclease.

13. (canceled)

14. A method of correcting or modifying frame shift mutations in an embryo comprising introducing into the embryo at least one guide RNA or DNA encoding at least one guide RNA, wherein the guide RNA targets a mutated allele with a detectable phenotype; and an RNA-guided endonuclease, or DNA encoding an RNA-guided endonuclease, wherein the endonuclease introduces a double-stranded break in a targeted site on the mutated allele resulting in the nonmosiac correction or modification of the frame shift mutation.

15. (canceled)

16. The method of claim 14, wherein the guide RNA is designed such that placement results in cleavage between two identical regions of nucleotides in the mutated allele, wherein this region defines the site of micro-homology.

17. (canceled)

18. The method of claim 14, wherein the guide RNA is designed to target at least one single nucleotide polymorphism specific for the mutation.

19. The method of claim 14, wherein the RNA-guided endonuclease is a Cas nuclease.

20. (canceled)

21. The method of claim 14, wherein the at least one guide RNA and the RNA-guided endonuclease are introduced to the embryo in a ribonucleoprotein complex.

22. (canceled)

23. The method of claim 14, further comprising genotyping an oocyte and sperm donor to determine the location of the mutated allele and the specific frame shift mutation on the mutated allele prior to the introduction of the at least one guide RNA or DNA encoding at least one guide RNA, and the RNA-guided endonuclease, or DNA encoding an RNA-guided endonuclease the embryo.

24. A method for correcting or modifying a frame shift mutation in an allele of a subject or in a cell of a subject comprising administering to the subject or contacting the cell with at least one type of vector comprising: (i) a first sequence encoding a guide RNA that hybridizes to the mutated allele; and (ii) a second sequence encoding at least one RNA-guided endonuclease, wherein the endonuclease introduces a double-stranded break in a targeted site on the mutated allele resulting in the correction or modification of the frame shift mutation, wherein the allele has a detectable phenotype.

25. (canceled)

26. The method of claim 24, wherein the guide RNA is designed such that placement results in cleavage between two identical regions of nucleotides in the mutated allele, wherein this region defines the site of micro-homology.

27. (canceled)

28. The method of claim 24, wherein the guide RNA is designed to target at least one single nucleotide polymorphism specific for the mutation.

29. The method of claim 24, wherein the RNA-guided endonuclease is a Cas nuclease.

30. (canceled)

31. (canceled)

32. The method of claim 24, wherein the at least one type of vector is at least one type of recombinant adeno-associated viral (AAV) vector.

33.-39. (canceled)

40. The method of claim 24, wherein the subject is selected from the group consisting of a fetus, a newborn, a child and an adult.


SEQUENCE LISTING (31 sequences)

SEQ ID NO: 1 - RNA, 101 nt, Artificial Sequence
  Synthetic Guide RNA
gugugucuuucuucuguacugguuuuagagcuagaaauagcaaguuaaaauaaggcuaguccguuaucaacuugaaaaaguggcaccgagucggugcuuuu

SEQ ID NO: 2 - DNA, 20 bp, homo sapiens
gtctttcttctgtactgagg

SEQ ID NO: 3 - DNA, 21 bp, homo sapiens
gtctttcttctgtactggagg

SEQ ID NO: 4 - DNA, 21 bp, Homo sapiens
ctgtaactgaggcttctgagg

SEQ ID NO: 5 - DNA, 19 bp, Homo sapiens
ctgtatgaggcttctgagg

SEQ ID NO: 6 - DNA, 25 bp, Homo sapiens
ctttctgcacacagagagactgagg

SEQ ID NO: 7 - DNA, 12 bp, Homo sapiens
ctgtaactgagg

SEQ ID NO: 8 - DNA, 15 bp, Homo sapiens
tttcttcttctgagg

SEQ ID NO: 9 - DNA, 14 bp, Homo sapiens
ctttcttctgtttt

SEQ ID NO: 10 - DNA, 16 bp, Homo sapiens
ctgtgtcttactgagg

SEQ ID NO: 11 - DNA, 17 bp, Homo sapiens
ctgtttttcttctgagg

SEQ ID NO: 12 - DNA, 27 bp, Homo sapiens
ctgtcttctgtgtctgtctttctgagg

SEQ ID NO: 13 - DNA, 12 bp, Homo sapiens
ctgaaactgagg

SEQ ID NO: 14 - DNA, 16 bp, Homo sapiens
ctgtgtcttactgagg

SEQ ID NO: 15 - DNA, 16 bp, Homo sapiens
cttctgtacctggagg

SEQ ID NO: 16 - DNA, 11 bp, Homo sapiens
ctgactggagg

SEQ ID NO: 17 - DNA, 10 bp, Homo sapiens
cttcctgagg

SEQ ID NO: 18 - DNA, 11 bp, Homo sapiens
tctgtctgagg

SEQ ID NO: 19 - DNA, 18 bp, Homo sapiens
agaaagatctttctgagg

SEQ ID NO: 20 - DNA, 13 bp, Homo sapiens
ctgtaactggagg

SEQ ID NO: 21 - DNA, 18 bp, Homo sapiens
agaaagatctttctgagg

SEQ ID NO: 22 - DNA, 14 bp, Homo sapiens
tcttctgtctgagg

SEQ ID NO: 23 - DNA, 23 bp, Homo sapiens
ccccagtacagaagagatacaca

SEQ ID NO: 24 - DNA, 20 bp, Artificial Sequence
  Synthetic Guide RNA
cagtacagaagaagacacaa

SEQ ID NO: 25 - DNA, 23 bp, Homo sapiens
tgtgtctttcttctgtactgagg

SEQ ID NO: 26 - DNA, 24 bp, Homo sapiens
tgtgtctttcttctgtactggagg

SEQ ID NO: 27 - DNA, 16 bp, Homo sapiens
ttcttctgtactgagg

SEQ ID NO: 28 - DNA, 23 bp, Artificial Sequence
  synthetic oligonucleotide
atatttgtatgttagaatctggg

SEQ ID NO: 29 - DNA, 23 bp, Artificial Sequence
  synthetic oligonucleotide
aagaatggtacactcgagggagg

SEQ ID NO: 30 - DNA, 23 bp, Artificial Sequence
  synthetic oligonucleotide
ggcgttcaaggacttattcaagg

SEQ ID NO: 31 - DNA, 23 bp, Artificial Sequence
  synthetic oligonucleotide
gaccgatgtattccacctacagg